(19) United States
(12) Patent Application Publication
(10) Pub. No.: US 2018/0170942 A1
(43) Pub. Date: Jun. 21, 2018

(54) POLYCYCLIC DERIVATIVES TARGETING RAL GTPASES AND THEIR THERAPEUTICAL APPLICATIONS

(71) Applicant: NANTBIO, INC., Culver City, CA (US)

(72) Inventors: Chunlin Tao, Newport Coast, CA (US); Chengzhi Yu, San Diego, CA (US); Tulay Polat, Tustin, CA (US); Chao Yan, Englewood, CO (US); Shahrooz Rabizadeh, Agoura Hills, CA (US); Daniel Theodorescu, Denver, CO (US)

(21) Appl. No.: 15/737,142

(22) PCT Filed: Jun. 16, 2016
(86) PCT No.: PCT/US2016/037783

§ 371 Date: Dec. 15, 2017

Related U.S. Application Data
(60) Provisional application No. 62/180,533, filed on Jun. 16, 2015.

(51) Int. Cl. C07D 491/052; C07D 471/04; C07D 519/00; A61P 35/04

(52) U.S. Cl. CPC C07D 491/052; A61P 35/04; C07D 519/00; C07D 471/04

(57) ABSTRACT

Contemplated compounds, compositions and methods are directed to Ral GTPase inhibitors with improved activity.

This application claims priority to U.S. Provisional Application 62/180,533, filed Jun. 16, 2015, which is incorporated by reference herein.

### FIELD OF THE INVENTION

The present invention is directed to various compounds, compositions, and methods for treatment of disorders, diseases, and pathologic conditions associated dysfunction of Ral GTPases, and especially to polycyclic compounds.

### BACKGROUND OF THE INVENTION

The background description includes information that may be useful in understanding the present invention. It is not an admission that any of the information provided herein is prior art or relevant to the presently claimed invention, or that any publication specifically or implicitly referenced is prior art.

All publications herein are incorporated by reference to the same extent as if each individual publication or patent application were specifically and individually indicated to incorporated by reference. Where a definition or use of a term in an incorporated reference is inconsistent or contrary to the definition of that term provided herein, the definition of that term provided herein applies and the definition of that term in the reference does not apply.

Mutations in RAS proto-oncogenes are found in approximately 20-30% of all human tumors, thereby placing the Ras variants among the most prevalent drivers of cancer. Due to the frequent involvement of Ras in the onset and progression of cancer, efficient inhibition of oncogenic Ras signaling has been considered the ‘Holy Grail’ in cancer therapy. Ras is mutated in cancer more frequently than any other oncogene. Hence, Ras has been a focus for the development of rationally designed anti-cancer drugs, yet to date none have been successfully developed. In 1989, several groups showed that posttranslational modification of Ras proteins by farnesyl lipids is essential for Ras membrane association and transformation. Farnesyltransferase (FTase) was then purified and characterized and shortly thereafter, a second prenyltransferase, geranylgeranyltransferase type I (GGTase-I), that modifies Ras with a geranylgeranyl lipid was discovered. GGTase-I inhibitors (GGTIs) were studied and at least one such inhibitor, GGTI-2417, has been shown to inhibit the in vitro growth and survival of the MiaPaCa2 pancreatic cell line. But, the inhibitory effects were modest and no clinical trials with GGTIs have followed. Unfortunately, mutated. Ras has proven to be an extremely difficult target for pharmacological modulation.

RalA and RalB are paralogs in the family of Ras monomeric G proteins that have approximately 85% amino acid identity, and play a role in the regulation of endocytosis, exocytosis, actin cytoskeletal dynamics, and transcription. Like Ras, Ral proteins have also been implicated in tumorigenesis and metastasis. Ral GTPases may be activated in a Ras-dependent manner, via several guanidine nucleotide exchange factors, including RalGDS. Activation of the Ral pathway has been shown to be a requirement for transformation of human cells (Cancer Cell 2004; 6: 171-83; Genes Dev 2002; 16:2045-57), and Ras-mediated transformation depends on activation of RalA (Cancer Cell 2005; 7:533-45). RalA and RalB also play a role in the transcriptional regulation of CD24, a metastasis-associated gene in bladder and other cancers (Cancer Res 2006; 66: 1917-22).

Thus, Ral GTPases present a compelling alternative therapeutic target for prevention and treatment of solid tumors and the metastasis of these cancers, and there exists a need for effective methods of inhibiting Ral GTPases for the treatment and prevention of cancer. More recently, selected compounds were disclosed with in vitro anticancer activities targeting Ral GTPases as described in WO 2013/096820, and a few of those compounds had EC₅₀ values in the micromolar range (Nature 2014 Nov. 20; 515 (7527):443-7). Similarly, WO 2016/007905 describes further related compounds as Ral GTPase inhibitors. Unfortunately, solubility of at least some of the reported compounds was problematic. Moreover, numerous compounds of the '820 and '905 publications had relatively low affinity to the target or high EC₅₀ values.

Therefore, even though various compounds, compositions, and methods are known as Ral GTPase inhibitors, all or all of them suffer from one or more disadvantages. Thus, there is still a need for improved compounds, compositions, and methods of Ral GTPase inhibition.

### SUMMARY OF THE INVENTION

The present invention provides molecules as described in Formula (I) that inhibit Ral GTPases, pharmaceutically-acceptable formulations, as well as therapeutic uses of these molecules to prevent or slow the growth and metastasis of cancer in a mammal.

In especially contemplated aspects, the molecules will have a general structure according to Formula I and may be present as pharmaceutically acceptable enantiomers, tautomers, diastereomers, racemates, and salts thereof

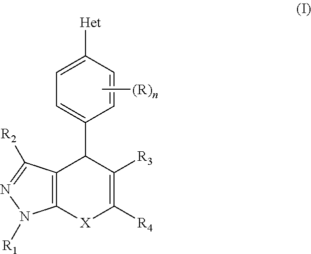

wherein R is independently selected from the group consisting of hydrogen, halogen, hydroxy, amino, cyano, —COOH, —SO₂NH₂, oxo, nitro, C₁-C₆ alkyl, C₂-C₆ alkenyl, C₂-C₆ alkynyl, C₃-C₆ cyclalkyl, C₅-C₆ aryl, substituted C₅-C₆ aryl, C₃-C₆ heteroaryl, substituted C₃-C₆ heteroaryl, C₂-C₆ alkoxycarbonyl, CONHSO₂R₅, CONR₅R₆, O—R₅, S—R₅, SO—R₅, SO₂—R₅, NHSO₂R₅, and NHCO₂R₅, and wherein n is an integer between 0 and 4; R₁ is selected from the group consisting of hydrogen, C₁-C₆ alkyl, C₃-C₆ alkenyl, C₅-C₆ aryl, substituted C₅-C₆ aryl, C₅-C₆ heteroaryl, substituted C₅-C₆ heteroaryl, and C₅-C₁₀ alkylaryl; R₂ is selected from the group consisting of hydrogen, halogen, amino, CN, COOH, C₁-C₁₀ alkyl, C₁-C₁₀ cycloalkyl, C₂-C₁₀ alkenyl, C₅-C₁₀ aryl, C₅-C₁₀ arylalkyl, substituted C₅-C₆ aryl, optionally substituted C₂-C₁₀ heteroaryl, optionally substituted heterocycloalkyl, optionally substituted heterocycloalkyl fused to aryl, C₁-C₆-alkoxy, C₂-C₆ alkanoyloxy, C₂-C₆ alkanoylamino, C₁-C₆ alkylthio, C₁-C₆ alkylsulfonyl, C₂-C₆ alkoxycarbonyl, CONR₅R₆, O—R₅, NHSO₂R₅ and NHCO₂R₅, wherein the heteroatoms in heteroaryl and heterocycloalkyl are selected from the group consisting of sulfur, nitrogen, and oxygen; R₃ and R₄ are independently CN, NO₂, NH₂, OH, COOH, CONR₅R₆, NHSO₂R₅, NHCOR₅, or NHCO₂R₅, or together form a 5-membered and 6-membered heterocycle in which the heteroatoms are selected from the group consisting of sulfur, nitrogen, and oxygen; X is O, NH, or NR₅; R₅ and R₆ are independently hydrogen, C₁-C₆ alkyl, C₃-C₆ alkenyl, C₅-C₆ aryl, C₂-C₁₀ heteroaryl, substituted C₅-C₁₀ aryl, substituted C₂-C₁₀ heteroaryl, each optionally substituted with one to three groups selected from the group consisting of halogen, C₁-C₆ alkyl, C₁-C₆ alkoxy, C₅-C₆ aryl, and C₃-C₆ heteroaryl, wherein the heteroatom in the heteroaryl is selected from the group consisting of sulfur, nitrogen, and oxygen; Het is a heteroaryl, optionally substituted with 1 to 4 substituents independently selected from the group consisting of C₁-C₆ alkyl, C₁-C₆ alkoxy, C₂-C₆ alkenyl, C₂-C₆ alkynyl, halogen, hydroxy, amino, amide, cyano, —COOH, —SO₂NH₂, oxo, nitro, alkoxycarbonyl, C₅-C₆ aryl, and C₂-C₆ heteroaryl, wherein Het has one or more heteroatoms selected from the group consisting of sulfur, nitrogen, and oxygen; and with the proviso that where X is O, R₃ is CN, R₄ is NH₂, and Het is imidazole, Het is substituted with alkyl or fused with an aryl ring.

Consequently, particularly contemplated compounds also include those having a structure according to Formulae (Ia)-(Ie)

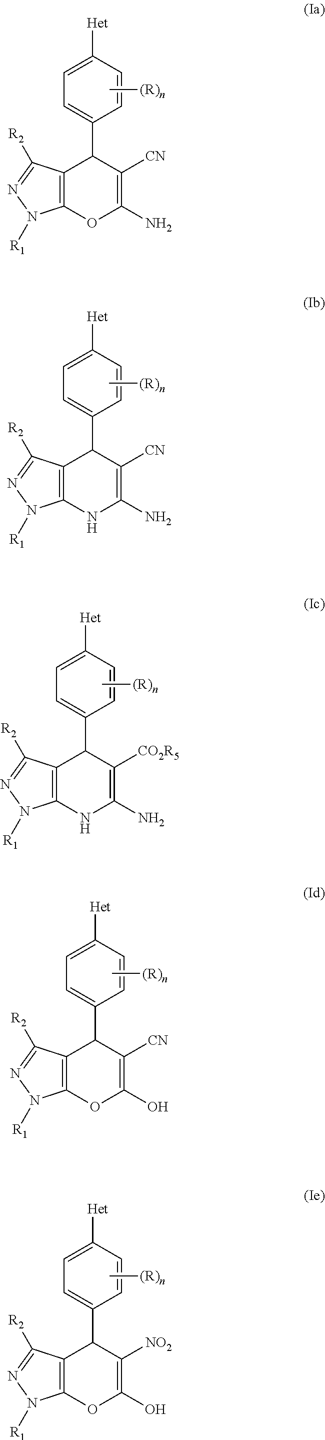

in which the radicals R, R₁, R₂, and Het are defined as noted above. Moreover, it is generally preferred that Het is a 5- or 6-membered ring with one or two N atoms as heteroatoms, or a group selected from

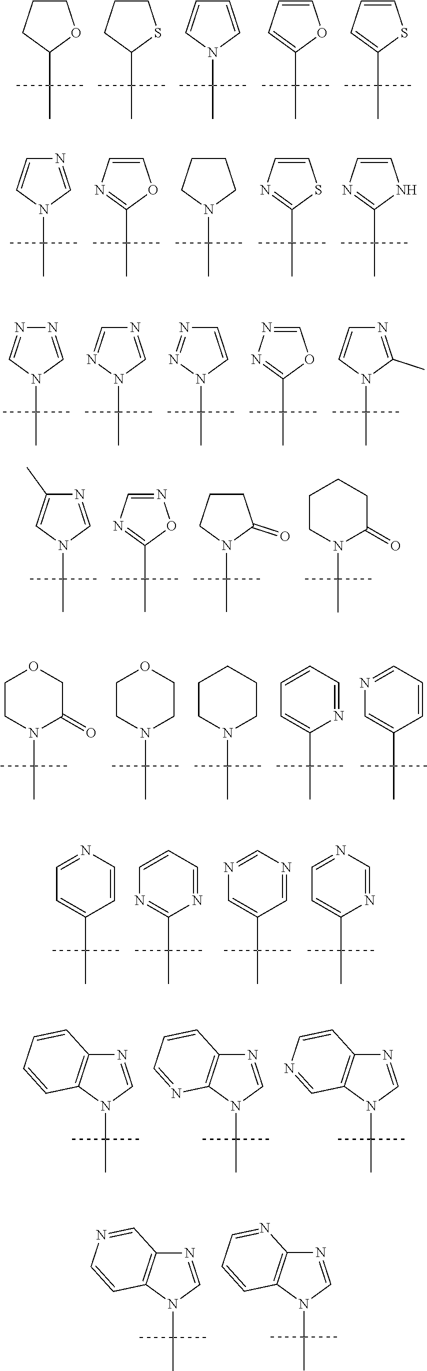

In further aspects, contemplated compounds also include those in which R₁ is hydrogen, C₁-C₆ alkyl, or optionally substituted C₅-C₆ aryl, and/or in which R₂ is hydrogen, C₁-C₁₀ alkyl, C₁-C₁₀ cycloalkyl, C₅-C₁₀ aryl, substituted C₅-C₆ aryl, optionally substituted C₂-C₁₀ heteroaryl, or optionally substituted heterocycloalkyl.

Additionally, the inventors contemplate, pharmaceutical composition that comprise one or more compounds presented herein, in combination with a pharmaceutically acceptable carrier. Most typically, the compound will be present in an amount effective to inhibit Ral. GTPase in a patient where the composition is administered to the patient, or in an amount effective to reduce growth of a cancer in a patient where the composition is administered to the patient, or in an amount effective to reduce incidence or multiplicity of metastases of a cancer in a patient where composition is administered to the patient. Preferably, such pharmaceutical compositions will be formulated for oral administration or for injection.

Consequently, the inventors also contemplate use of contemplated compounds and compositions for inhibition of at least one of RalA or RalB and for the manufacture of a medicament for treatment of a disease associated with at least one of RalA or RalB. Suitable diseases include cancer (e.g., pancreas, prostate, lung, bladder, colon cancer), and particularly metastatic cancer.

Thus, and viewed from a different perspective, the inventors contemplate methods of preventing or treating cancer that include a step of administering to an individual in need thereof a therapeutically effective amount of a compound contemplated herein in an amount effective to inhibit a Ral GTPase in the cancer, or a method of preventing or treating metastasis of a cancer in an individual that includes a step of administering to the individual in need thereof a therapeutically effective amount of contemplated compounds in an amount effective to inhibit a Ral GTPase in the cancer.

In a further aspect of the inventive subject matter, the inventors also contemplate a method of inhibiting RalA and/or RalB in which RalA and/or RalB is/are contacted with a compound presented herein in an amount effective to inhibit RalA and/or RalB. For example, the step of contacting may be performed in vitro or in vivo, and the effective amount is most preferably less than 5 microM, or less than 1 microM. It is still further contempated that the inhibition of RalA and/or RalB is inhibition of the GDP-bound forms of RalA and/or RalB.

Various objects, features, aspects and advantages of the inventive subject matter will become more apparent from the following detailed description of preferred embodiments.

### DETAILED DESCRIPTION OF THE INVENTION

The inventors have now discovered various Ral GTPase inhibitors with substantially improved activity that can be used in compositions and methods of treatment and prevention of cancer growth and metastasis. In a generally contemplated aspect of the inventive subject matter, compounds will have a structure according to general Formula (I)

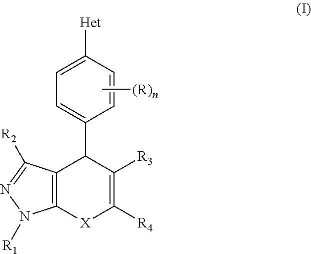

and all pharmaceutically acceptable enantiomers, tautomers, diastereomers, racemates, and salts thereof, wherein:

R is independently selected from the group consisting of hydrogen, halogen, hydroxy, amino, cyano, —COOH, —SO₂NH₂, oxo, nitro, C₁-C₆ alkyl, C₂-C₆ alkenyl, C₂-C₆ alkynyl, C₃-C₆ cyclalkyl, C₅-C₆ aryl, substituted C₅-C₆ aryl, C₃-C₆ heteroaryl, substituted C₃-C₆ heteroaryl, C₂-C₆ alkoxycarbonyl, CONHSO₂R₅ and CONR₅R₆, O—R₅, S—R₅, SO—R₅, SO₂—R₅, NHSO₂R₅ and NHCO₂R₅, and wherein n is an integer between 0 and 4.

R₁ is selected from hydrogen, C₁-C₆ alkyl, C₃-C₆ alkenyl, C₅-C₆ aryl, substituted C₅-C₆ aryl, C₅-C₆ heteroaryl, substituted C₅-C₆ heteroaryl, C₅-C₁₀ alkylaryl.

R₂ is selected from hydrogen, halogen, amino, CN, COOH, C₁-C₁₀ alkyl, C₁-C₁₀ cycloalkyl, C₂-C₁₀ alkenyl, C₅-C₁₀ aryl, C₅-C₁₀ arylalkyl, substituted C₅-C₆ aryl, C₂-C₁₀ optionally substituted heteroaryl, optionally substituted heterocycloalkyl, optionally substituted heterocycloalkyl fused to aryl, substituted C₂-C₁₀ heteroaryl, C₁-C₆-alkoxy, C₂-C₆ alkanoyloxy, C₂-C₆ alkanoylamino, C alkylthio, C₁-C₆ alkylsulfonyl, C₂-C₆ alkoxycarbonyl, CONR₅R₆, O—R₅, NHSO₂R₅ and NHCO₂R₅, wherein the heteroatoms in heteroaryl and heterocycloalkyl are selected from the group consisting of sulfur, nitrogen, and oxygen.

R₃ and R₄ are independently selected from CN, NO₂, NH₂, OH, COOH, CONR₅R₆, NHSO₂R₅, NHCOR₅, NHCO₂R₅, and together form a 5-membered and 6-membered heterocycle in which the heteroatom(s) is/are selected from the group consisting of sulfur, nitrogen, and oxygen.

X is selected from O, NH, and NR₅.

R₅ and R₆ are independently selected from hydrogen, C₁-C₆ alkyl, C₃-C₆ alkenyl, C₅-C₆ aryl, optionally substituted C₅-C₁₀ aryl, optionally substituted C₂-C₁₀ heteroaryl, wherein optional substitution is with one to three groups selected from halogen, C₁-C₆ alkyl, C₁-C₆ alkoxy, C₅-C₆ aryl, and C₃-C₆ heteroaryl, wherein the heteroatom(s) in heteroaryl is/are independently selected from the group consisting of sulfur, nitrogen, and oxygen.

Het is selected from any heterocycle or heteroaryl, optionally substituted with from 0 to 4 substituents independently chosen from: (i) C₁-C₆ alkyl, C alkoxy, C₂-C₆ alkenyl, C₂-C₆ alkynyl; (ii) halogen, hydroxy, amino, amide, cyano, —COOH, —SO₂NH₂, oxo, nitro and alkoxycarbonyl; (iii) C₅-C₆ aryl; and (iv) C₂-C₆ heteroaryl, wherein the heteroatom(s) in Het is/are independently selected from the group consisting of sulfur, nitrogen, and oxygen. In further contemplated compounds, and especially where X is O, R₃ is CN, R₄ is NH₂, and Het is imidazole, Het will be substituted with alkyl or will be fused with an aryl ring.

As used herein, the term “halo” or “halogen” refers to fluorine, chlorine, bromine or iodine.

The term “alkyl” herein alone or as part of another group refers to a monovalent alkane (hydrocarbon) derived radical containing from 1 to 12 carbon atoms unless otherwise defined. Alkyl groups may be substituted at any available point of attachment. An alkyl group substituted with another alkyl group is also referred to as a “branched alkyl group”. Exemplary alkyl groups include methyl, ethyl, propyl, isopropyl, n-butyl, t-butyl, isobutyl, pentyl, hexyl, isohexyl, heptyl, dimethylpentyl, octyl, 2,2,4-trimethylpentyl, nonyl, decyl, undecyl, dodecyl, and the like.

Exemplary substituents for radicals designated as “optionally substituted” include one or more of the following groups: alkyl, aryl, halo (such as F, Cl, Br, I), haloalkyl (such as CCl₃ or CF₃), alkoxy, alkylthio, hydroxy, carboxy (—COOH), alkyloxycarbonyl (—C(O)R), alkylcarbonyloxy (—OCOR), amino (—NH₂), carbamoyl (—NHCOOR— or —OCONHR—), urea (—NHCONHR—) or thiol (—SH). In some embodiments of the present invention, alkyl groups are substituted with, for example, amino, or heterocycloalkyl, such as morpholine, piperazine, piperidine, azetidine, hydroxyl, methoxy, or a heteroaryl group, such as pyrrolidine.

The term “cycloalkyl” herein alone or as part of another group refers to fully saturated and partially unsaturated hydrocarbon rings of 3 to 9, preferably 3 to 7 carbon atoms. The examples include cyclopropyl, cyclobutyl, cyclopentyl and cyclohexyl, and like. Further, a cycloalkyl may be substituted. For example, a substituted cycloalkyl refers to such rings having one, two, or three substituents, selected from the group consisting of halo, alkyl, substituted alkyl, alkenyl, alkynyl, nitro, cyano, oxo (═O), hydroxy, alkoxy, thioalkyl, —CO₂H, —C(═O)H, CO₂-alkyl, —C(═O)alkyl, keto, ═N—OH, ═N—O-alkyl, aryl, heteroaryl, heterocyclo, —NR′R″, —C(═O)NR′R″, —CO₂NR′R″, —C(═O)NR′R″, —NR′CO₂R″, —NR′C(═O)R″, —SO₂NR′R″, and —NR′SO₂R″, wherein each of R and R″ are independently selected from hydrogen, alkyl, substituted alkyl, and cycloalkyl, or R′ and R″ together form a heterocyclo or heteroaryl ring.

The term ‘alkenyl” herein alone or as part of another group refers to a hydrocarbon radical straight, branched or cyclic containing from 2 to 12 carbon atoms and at least one carbon to carbon double bond. Examples of such groups include the vinyl, allyl, 1-propenyl, isopropenyl, 2-methyl-1-propenyl, 1-butenyl, 2-butenyl, 3-butenyl, 1-pentenyl, 2-pentenyl, 3-pentenyl, 4-pentenyl, 1-hexenyl, 2-hexenyl, 3-hexenyl, 4-hexenyl, 5-hexenyl, 1-heptenyl, and like. Alkenyl groups may also be substituted at any available point of attachment. Exemplary substituents for alkenyl groups include those listed above for alkyl groups, and especially include C3 to C7 cycloalkyl groups such as cyclopropyl, cyclopentyl and cyclohexyl, which may be further substituted with, for example, amino, oxo, hydroxyl, etc.

The term “alkynyl” refers to straight or branched chain alkyne groups, which have one or more unsaturated carbon-carbon bonds, at least one of which is a triple bond. Alkynyl groups include C2-C8 alkynyl, C2-C6 alkynyl and C2-C4 alkynyl groups, which have from 2 to 8, 2 to 6 or 2 to 4 carbon atoms, respectively. Illustrative of the alkynyl group include ethenyl, propenyl, isopropenyl, butenyl, isobutenyl, pentenyl, and hexenyl. Alkynyl groups may also be substituted at any available point of attachment. Exemplary substituents for alkynyl groups include those listed above for alkyl groups such as amino, alkylamino, etc. The numbers in the subscript after the symbol “C” define the number of carbon atoms a particular group can contain.

The term “alkoxy” alone or as part of another group denotes an alkyl group as described above bonded through an oxygen linkage (—O—). Preferred alkoxy groups have from 1 to 8 carbon atoms. Examples of such groups include the methoxy, ethoxy, n-propoxy, isopropoxy, n-butoxy, isobutoxy, sec-butoxy, tert-butoxy, n-pentyloxy, isopentyloxy, n-hexyloxy, cyclohexyloxy, n-heptyloxy, n-octyloxy and 2-ethylhexyloxy.

The term “alkylthio” refers to an alkyl group as described above attached via a sulfur bridge. Preferred alkoxy and alkylthio groups are those in which an alkyl group is attached via the heteroatom bridge. Preferred alkylthio groups have from 1 to 8 carbon atoms. Examples of such groups include the methylthio, ethylthio, n-propythiol, n-butylthiol, and like.

The term “oxo,” as used herein, refers to a keto (C═O) group. An oxo group that is a substituent of a nonaromatic carbon atom results in a conversion of —CH₂— to —C(═O)—.

The term “alkoxycarbonyl” herein alone or as part of another group denotes an alkoxy group bonded through a carbonyl group. An alkoxycarbonyl radical is represented by the formula: —C(O)OR, where the R group is a straight or branched C1-C6 alkyl group, cycloalkyl, aryl, or heteroaryl.

The term “alkylcarbonyl” herein alone or as part of another group denotes an alkyl group bonded through a carbonyl group. An alkoxycarbonyl radical is represented by the formula: —C(O)R, where the R group is a straight or branched C1-C6 alkyl group, cycloalkyl, aryl, or heteroaryl.

The term “alkanoyloxy” herein alone or as part of another group denotes an RCOO— group bonded through a single bond. An alkanoyloxy radical is represented by the formula RCOO—, where the R group is a straight or branched C1-C6 alkyl group, cycloalkyl, aryl, or heteroaryl.

The term “alkanoylamino” herein alone or as part of another group denotes an RCONH— group bonded through a single bond. An alkanoylamino radical is represented by the formula RCONH—, where the R group is a straight or branched C1-C6 alkyl group, cycloalkyl, aryl, or heteroaryl.

The term “arylalkyl” herein alone or as part of another group denotes an aromatic ring bonded through an alkyl group (such as benzyl) as described above.

The term “aryl” herein alone or as part of another group refers to monocyclic or bicyclic aromatic rings, e.g. phenyl, substituted phenyl and the like, as well as groups which are fused, e.g., napthyl, phenanthrenyl and the like. An aryl group thus contains at least one ring having at least 6 atoms, with up to five such rings being present, containing up to 20 atoms therein, with alternating (resonating) double bonds between adjacent carbon atoms or suitable heteroatoms. Aryl groups may optionally be substituted with one or more groups including, but not limited to halogen such as I, Br, F, or Cl; alkyl, such as methyl, ethyl, propyl, alkoxy, such as methoxy or ethoxy, hydroxy, carboxy, carbamoyl, alkyloxycarbonyl, nitro, alkenyloxy, trifluoromethyl, amino, cycloalkyl, aryl, heteroaryl, cyano, alkyl S(O)ₘ (m=0, 1, 2), or thiol.

The term “aromatic” refers to a cyclically conjugated molecular entity with a stability, due to delocalization, significantly greater than that of a hypothetical localized structure, such as the Kekule structure.

The term “amino” herein alone or as part of another group refers to —NH2. An “amino” may optionally be substituted with one or two substituents, which may be the same or different, such as alkyl, aryl, arylalkyl, alkenyl, alkynyl, heteroaryl, heteroarylalkyl, cycloheteroalkyl, cycloheteroalkylalkyl, cycloalkyl, cycloalkylalkyl, haloalkyl, hydroxyalkyl, alkoxyalkyl, thioalkyl, carbonyl or carboxyl. These substituents may be further substituted with a carboxylic acid, any of the alkyl or aryl substituents set out herein. In some embodiments, the amino groups are substituted with carboxyl or carbonyl to form N-acyl or N-carbamoyl derivatives.

The term “alkylsulfonyl” refers to groups of the formula (SO₂)-alkyl, in which the sulfur atom is the point of attachment. Preferably, alkylsulfonyl groups include C1-C6 alkylsulfonyl groups, which have from 1 to 6 carbon atoms. Methylsulfonyl is one representative alkylsulfonyl group.

The term “heteroatom” refers to any atom other than carbon, for example, N, O, or S.

The term “heteroaryl” herein alone or as part of another group refers to substituted and unsubstituted aromatic 5 or 6 membered monocyclic groups, 9 or 10 membered bicyclic groups, and 11 to 14 membered tricyclic groups which have at least one heteroatom (O, S or N) in at least one of the rings. Each ring of the heteroaryl group containing a heteroatom can contain one or two oxygen or sulfur atoms and/or from one to four nitrogen atoms provided that the total number of heteroatoms in each ring is four or less and each ring has at least one carbon atom.

The term “heterocyclic” or “heterocycloalkyl” herein alone or as part of another group refers to a cycloalkyl group (nonaromatic) in which one of the carbon atoms in the ring is replaced by a heteroatom selected from O, S or N. The “heterocycle” has from 1 to 3 fused, pendant or spiro rings, at least one of which is a heterocyclic ring (i.e., one or more ring atoms is a heteroatom, with the remaining ring atoms being carbon). The heterocyclic ring may be optionally substituted which means that the heterocyclic ring may be substituted at one or more substitutable ring positions by one or more groups independently selected from alkyl (preferably lower alkyl), heterocycloalkyl, heteroaryl, alkoxy (preferably lower alkoxy), nitro, monoalkylamino (preferably a lower alkylamino), dialkylamino (preferably a alkylamino), cyano, halo, haloalkyl (preferably trifluoromethyl), alkanoyl, aminocarbonyl, monoalkylaminocarbonyl, dialkylaminocarbonyl, alkyl amido (preferably lower alkyl amido), alkoxyalkyl (preferably a lower alkoxy; lower alkyl), alkoxycarbonyl (preferably a lower alkoxycarbonyl), alkylcarbonyloxy (preferably a lower alkylcarbonyloxy) and aryl (preferably phenyl), said aryl being optionally substituted by halo, lower alkyl and lower alkoxy groups. A heterocyclic group may generally be linked via any ring or substituent atom, provided that a stable compound results. N-linked heterocyclic groups are linked via a component nitrogen atom.

Typically, a heterocyclic ring comprises 1-4 heteroatoms; within certain embodiments each heterocyclic ring has 1 or 2 heteroatoms per ring. Each heterocyclic ring generally contains from 3 to 8 ring members (rings having from to 7 ring members are recited in certain embodiments), and heterocycles comprising fused, pendant or spiro rings typically contain from 9 to 14 ring members which consists of carbon atoms and contains one, two, or three heteroatoms selected from nitrogen, oxygen and/or sulfur. Examples of “heterocyclic” or “heterocycloalkyl” groups include piperazine, piperidine, morpholine, thiomorpholine, pyrrolidine, imidazolidine and thiazolide.

The term “substituent,” as used herein, refers to a molecular moiety that is covalently bonded to an atom within a molecule of interest. For example, a “ring substituent” may be a moiety such as a halogen, alkyl group, haloalkyl group or other group discussed herein that is covalently bonded to an atom (preferably a carbon or nitrogen atom) that is a ring member.

The term “optionally substituted” as used herein means that the aryl, heterocyclyl, or other group may be substituted at one or more substitutable positions by one or more groups independently selected from alkyl (preferably lower alkyl), alkoxy (preferably lower alkoxy), nitro, monoalkylamino (preferably with one to six carbons), dialkylamino (preferably with one to six carbons), cyano, halo, haloalkyl (preferably trifluoromethyl), alkanoyl, aminocarbonyl, monoalkylaminocarbonyl, dialkylaminocarbonyl, alkyl amido (preferably lower alkyl amido), alkoxyalkyl (preferably a lower alkoxy and lower alkyl), alkoxycarbonyl (preferably a lower alkoxycarbonyl), alkylcarbonyloxy (preferably a lower alkylcarbonyloxy) and aryl (preferably phenyl), said aryl being optionally substituted by halo, lower alkyl and lower alkoxy groups. Optional substitution is also indicated by the phrase “substituted with from 0 to X substituents,” where X is the maximum number of possible substituents. Certain optionally substituted groups are substituted with from 0 to 2, 3 or 4 independently selected substituents.

A dash (“-”) that is not between two letters or symbols is used to indicate a point of the attachment for a substituent. For example, —CONH2 is attached through the carbon atom. A dashed cycle that locates inside of a heterocyle ring is used to indicate a conjugated system. The bonds between two atoms may be single bond or double bond.

The term “tautomer” includes both tautomeric forms A and B, or C and D, of a compound of formula I as well as a mixture thereof,

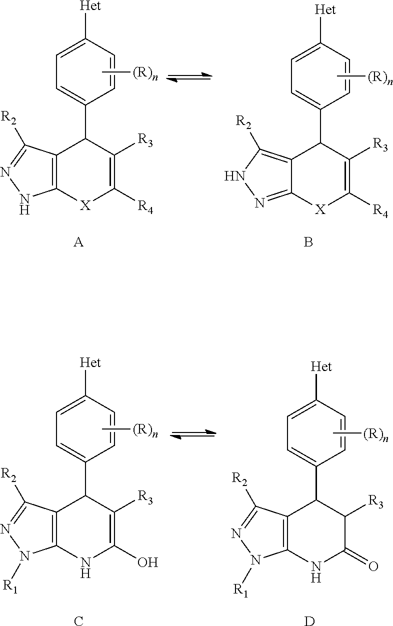

It is possible to use both A and B, or both C and D, a pure tautomer and any mixture thereof, in particular compositions according to the invention.

The term “therapeutically effective amount” refers to the amount of the compound or pharmaceutical composition that will elicit a biological or medical response of a tissue, system, animal or human that is being sought by the researcher, veterinarian, medical doctor or other clinician, e.g., reduction of tumor growth and/or burden, reduction of occurrence or multiplicity of metastasis, reduction of morbidity and/or mortality.

The term “pharmaceutically acceptable” refers to the fact that the carrier, diluent or excipient must be compatible with the other ingredients of the formulation and not deleterious to the recipient thereof.

The terms “administration of a compound” or “administering a compound” refer to the act of providing a compound of the invention or pharmaceutical composition to the subject in need of treatment. Where two or more compounds ad administered, co-administration is typically preferred with the co-administration being either via a combination formulation, or via parallel or subsequent administration of the two compounds. Most typically sequential co-administration will be performed such that the first compound is present in the patient's body in measurable quantities when the second compound is administered.

The term “protected” refers that the group is in modified form to preclude undesired side reactions at the protected site. Suitable protecting groups for the compounds of the present invention will be recognized from the present application taking into account the level of skill in the art, and with reference to standard textbooks, such as Greene, T. W. et al., Protective Groups in Organic Synthesis, John Wiley & Sons, New York (1999).

The term “pharmaceutically acceptable salt” of a compound recited herein is an acid or base salt that is suitable for use in contact with the tissues of human beings or animals without excessive toxicity or carcinogenicity, and preferably without irritation, allergic response, or other problem or complication. Such salts include mineral and organic acid salts of basic residues such as amines, as well as alkali or organic salts of acidic residues such as carboxylic acids. Specific pharmaceutical salts include, but are not limited to, salts of acids such as hydrochloric, phosphoric, hydrobromic, malic, glycolic, fumaric, sulfuric, sulfamic, sulfanilic, formic, toluenesulfonic, methanesulfonic, benzene sulfonic, ethane disulfonic, 2-hydroxyethylsulfonic, nitric, benzoic, 2-acetoxybenzoic, citric, tartaric, lactic, stearic, salicylic, glutamic, ascorbic, pamoic, succinic, fumaric, maleic, propionic, hydroxymaleic, hydroiodic, phenylacetic, alkanoic such as acetic, HOOC—(CH₂)ₙ—COOH where n is 0-4, and the like.

Similarly, pharmaceutically acceptable cations include, but are not limited to sodium, potassium, calcium, aluminum, lithium and ammonium. Those of ordinary skill in the art will recognize further pharmaceutically acceptable salts for the compounds provided herein. In general, a pharmaceutically acceptable acid or base salt can be synthesized from a parent compound that contains a basic or acidic moiety by any conventional chemical method. Briefly, such salts can be prepared by reacting the free acid or base forms of these compounds with a stoichiometric amount of the appropriate base or acid in water or in an organic solvent, or in a mixture of the two; generally, the use of nonaqueous media, such as ether, ethyl acetate, ethanol, isopropanol or acetonitrile, is preferred. Lists of suitable salts are found in at page 1418 of Remington's Pharmaceutical Sciences, 17th ed., Mack Publishing Company, Easton, Pa., 1985.

The term “solvate” refers to the compound formed by the interaction of a solvent and a compound. Suitable solvates are pharmaceutically acceptable solvates, such as hydrates, including monohydrates and hemi-hydrates.

Exemplary Contemplated Compounds

In one particularly contemplated aspect of the inventive subject matter, compounds will have a structure according to Formula (Ia):

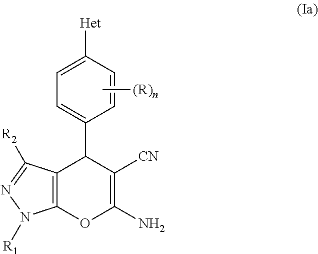

and pharmaceutically acceptable enantiomers, tautomers, diastereomers, racemates, and salts thereof, wherein:

R is independently selected from the group consisting of hydrogen, halogen, hydroxy, amino, cyano, —COOH, —SO₂NH₂, oxo, nitro, C₁-C₆ alkyl, C₂-C₆ alkenyl, C₂-C₆ alkynyl, C₃-C₆ cyclalkyl, C₅-C₆ aryl, substituted C₅-C₆ aryl, C₃-C₆ heteroaryl, substituted C₃-C₆ heteroaryl, C₂-C₆ alkoxycarbonyl, CONHSO₂R₅ and CONR₅R₆, O—R₅, S—R₅, SO—R₅, SO₂—R₅, NHSO₂R₅ and NHCO₂R₅, and wherein n is an integer between 0 and 4.

R₁ is selected from hydrogen, C₁-C₆ alkyl, C₃-C₆ alkenyl, C₅-C₆ aryl, substituted C₅-C₆ aryl, C₅-C₆ heteroaryl, substituted C₅-C₆ heteroaryl, C₅-C₁₀ alkylaryl.

R₂ is selected from hydrogen, halogen, amino, CN, COOH, C₁-C₁₀ alkyl, C₁-C₁₀ cycloalkyl, C₂-C₁₀ alkenyl, C₅-C₁₀ aryl, C₅-C₁₀ arylalkyl, substituted C₅-C₆ aryl, C₂-C₁₀ optionally substituted heteroaryl, optionally substituted heterocycloalkyl, optionally substituted heterocycloalkyl fused to aryl, substituted C₂-C₁₀ heteroaryl, C₁-C₆-alkoxy, C₂-C₆ alkanoyloxy, C₂-C₆ alkanoylamino, C₁-C₆ alkylthio, C₁-C₆ alkylsulfonyl, C₂-C₆ alkoxycarbonyl, CONR₅R₆, O—R₅, NHSO₂R₅ and NHCO₂R₅, wherein the heteroatoms in heteroaryl and heterocycloalkyl are selected from the group consisting of sulfur, nitrogen, and oxygen.

R₅ and R₆ are independently selected from hydrogen, C₁-C₆ alkyl, C₃-C₆ alkenyl, C₅-C₆ aryl, optionally substituted C₅-C₁₀ aryl, optionally substituted C₂-C₁₀ heteroaryl, wherein optional substitution is with one to three groups selected from halogen, C₁-C₆ alkyl, C₁-C₆ alkoxy, C₅-C₆ aryl, and C₃-C₆ heteroaryl, wherein the heteroatom(s) in heteroaryl is/are independently selected from the group consisting of sulfur, nitrogen, and oxygen.

Het is selected from any heterocycle or heteroaryl, optionally substituted with from 0 to 4 substituents independently chosen from: (i) C₁-C₆ alkyl, C₁-C₆ alkoxy, C₂-C₆ alkenyl, C₂-C₆ alkynyl; (ii) halogen, hydroxy, amino, amide, cyano, —COOH, —SO₂NH₂, oxo, nitro and alkoxycarbonyl; (iii) C₅-C₆ aryl; and (iv) C₂-C₆ heteroaryl, wherein the heteroatom(s) in Het is/are independently selected from the group consisting of sulfur, nitrogen, and oxygen.

In another contemplated aspect, compounds according to the inventive subject matter will have a structure according to Formula (Ib):

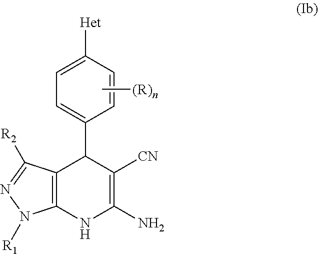

and pharmaceutically acceptable enantiomers, tautomers, diastereomers, racemates, and salts thereof, wherein:

R is independently selected from the group consisting of hydrogen, halogen, hydroxy, amino, cyano, —COOH, —SO₂NH₂, oxo, nitro, C₁-C₆ alkyl, C₂-C₆ alkenyl, C₂-C₆ alkynyl, C₃-C₆ cyclalkyl, C₅-C₆ aryl, substituted C₅-C₆ aryl, C₃-C₆ heteroaryl, substituted C₃-C₆ heteroaryl, C₂-C₆ alkoxycarbonyl, CONHSO₂R₅ and CONR₅R₆, O—R₅, S—R₅, SO—R₅, SO₂—R₅, NHSO₂R₅ and NHCO₂R₅, and wherein n is an integer between 0 and 4.

R₁ is selected from hydrogen, C₁-C₆ alkyl, C₃-C₆ alkenyl, C₅-C₆ aryl, substituted C₅-C₆ aryl, C₅-C₆ heteroaryl, substituted C₅-C₆ heteroaryl, C₅-C₁₀ alkylaryl.

R₂ is selected from hydrogen, halogen, amino, CN, COOH, C₁-C₁₀ alkyl, C₁-C₁₀ cycloalkyl, C₂-C₁₀ alkenyl, C₅-C₁₀ aryl, C₅-C₁₀ arylalkyl, substituted C₅-C₆ aryl, C₂-C₁₀ optionally substituted heteroaryl, optionally substituted heterocycloalkyl, optionally substituted heterocycloalkyl fused to aryl, substituted C₂-C₁₀ heteroaryl, C₁-C₆-alkoxy, C₂-C₆ alkanoyloxy, C₂-C₆ alkanoylamino, C alkylthio, C₁-C₆ alkylsulfonyl, C₂-C₆ alkoxycarbonyl, CONR₅R₆, O—R₅, NHSO₂R₅ and NHCO₂R₅, wherein the heteroatoms in heteroaryl and heterocycloalkyl are selected from the group consisting of sulfur, nitrogen, and oxygen.

R₅ and R₆ are independently selected from hydrogen, C₁-C₆ alkyl, C₃-C₆ alkenyl, C₅-C₆ aryl, optionally substituted C₅-C₁₀ aryl, optionally substituted C₂-C₁₀ heteroaryl, wherein optional substitution is with one to three groups selected from halogen, C₁-C₆ alkyl, C₁-C₆ alkoxy, C₅-C₆ aryl, and C₃-C₆ heteroaryl, wherein the heteroatom(s) in heteroaryl is/are independently selected from the group consisting of sulfur, nitrogen, and oxygen.

Het is selected from any heterocycle or heteroaryl, optionally substituted with from 0 to 4 substituents independently chosen from: (i) C₁-C₆ alkyl, C₁-C₆ alkoxy, C₂-C₆ alkenyl, C₂-C₆ alkynyl; (ii) halogen, hydroxy, amino, amide, cyano, —COOH, —SO₂NH₂, oxo, nitro and alkoxycarbonyl; (iii) C₅-C₆ aryl; and (iv) C₂-C₆ heteroaryl, wherein the heteroatom(s) in Het is/are independently selected from the group consisting of sulfur, nitrogen, and oxygen.

In yet another contemplated aspect, compounds according to the inventive subject matter will have a structure according to Formula (Ic):

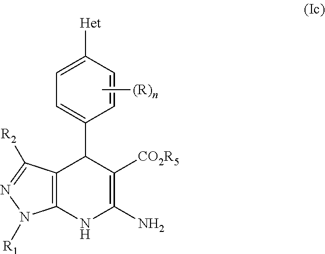

and pharmaceutically acceptable enantiomers, tautomers, diastereomers, racemates, and salts thereof, wherein:

R is independently selected from the group consisting of hydrogen, halogen, hydroxy, amino, cyano, —COOH, —SO₂NH₂, oxo, nitro, C₁-C₆ alkyl, C₂-C₆ alkenyl, C₂-C₆ alkynyl, C₃-C₆ cyclalkyl, C₅-C₆ aryl, substituted C₅-C₆ aryl, C₃-C₆ heteroaryl, substituted C₃-C₆ heteroaryl, C₂-C₆ alkoxycarbonyl, CONHSO₂R₅ and CONR₅R₆, O—R₅, S—R₅, SO—R₅, SO₂—R₅, NHSO₂R₅ and NHCO₂R₅, and wherein n is an integer between 0 and 4.

R₁ is selected from hydrogen, C₁-C₆ alkyl, C₃-C₆ alkenyl, C₅-C₆ aryl, substituted C₅-C₆ aryl, C₅-C₆ heteroaryl, substituted C₅-C₆ heteroaryl, C₅-C₁₀ alkylaryl.

R₂ is selected from hydrogen, halogen, amino, CN, COOH, C₁-C₁₀ alkyl, C₁-C₁₀ cycloalkyl, C₂-C₁₀ alkenyl, C₅-C₁₀ aryl, C₅-C₁₀ arylalkyl, substituted C₅-C₆ aryl, C₂-C₁₀ optionally substituted heteroaryl, optionally substituted heterocycloalkyl, optionally substituted heterocycloalkyl fused to aryl, substituted C₂-C₁₀ heteroaryl, C₁-C₆-alkoxy, C₂-C₆ alkanoyloxy, C₂-C₆ alkanoylamino, C alkylthio, C₁-C₆ alkylsulfonyl, C₂-C₆ alkoxycarbonyl, CONR₅R₆, O—R₅, NHSO₂R₅ and NHCO₂R₅, wherein the heteroatoms in heteroaryl and heterocycloalkyl are selected from the group consisting of sulfur, nitrogen, and oxygen.

R₅ and R₆ are independently selected from hydrogen, C₁-C₆ alkyl, C₃-C₆ alkenyl, C₅-C₆ aryl, optionally substituted C₅-C₁₀ aryl, optionally substituted C₂-C₁₀ heteroaryl, wherein optional substitution is with one to three groups selected from halogen, C₁-C₆ alkyl, C₁-C₆ alkoxy, C₅-C₆ aryl, and C₃-C₆ heteroaryl, wherein the heteroatom(s) in heteroaryl is/are independently selected from the group consisting of sulfur, nitrogen, and oxygen.

Het is selected from any heterocycle or heteroaryl, optionally substituted with from 0 to 4 substituents independently chosen from: (i) C₁-C₆ alkyl, C₁-C₆ alkoxy, C₂-C₆ alkenyl, C₂-C₆ alkynyl; (ii) halogen, hydroxy, amino, amide, cyano, —COOH, —SO₂NH₂, oxo, nitro and alkoxycarbonyl; (iii) C₅-C₆ aryl; and (iv) C₂-C₆ heteroaryl, wherein the heteroatom(s) in Het is/are independently selected from the group consisting of sulfur, nitrogen, and oxygen.

In a still further contemplated aspect, compounds according to the inventive subject matter will have a structure according to Formula (Id):

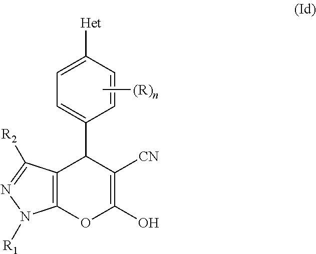

and pharmaceutically acceptable enantiomers, tautomers, diastereomers, racemates, and salts thereof, wherein:

R is independently selected from the group consisting of hydrogen, halogen, hydroxy, amino, cyano, —COOH, —SO₂NH₂, oxo, nitro, C₁-C₆ alkyl, C₂-C₆ alkenyl, C₂-C₆ alkynyl, C₃-C₆ cyclalkyl, C₅-C₆ aryl, substituted C₅-C₆ aryl, C₃-C₆ heteroaryl, substituted C₃-C₆ heteroaryl, C₂-C₆ alkoxycarbonyl, CONHSO₂R₅ and CONR₅R₆, O—R₅, S—R₅, SO—R₅, SO₂—R₅, NHSO₂R₅ and NHCO₂R₅, and wherein n is an integer between 0 and 4.

R₁ is selected from hydrogen, C₁-C₆ alkyl, C₃-C₆ alkenyl, C₅-C₆ aryl, substituted C₅-C₆ aryl, C₅-C₆ heteroaryl, substituted C₅-C₆ heteroaryl, C₅-C₁₀ alkylaryl.

R₂ is selected from hydrogen, halogen, amino, CN, COOH, C₁-C₁₀ alkyl, C₁-C₁₀ cycloalkyl, C₂-C₁₀ alkenyl, C₅-C₁₀ aryl, C₅-C₁₀ arylalkyl, substituted C₅-C₆ aryl, C₂-C₁₀ optionally substituted heteroaryl, optionally substituted heterocycloalkyl, optionally substituted heterocycloalkyl fused to aryl, substituted C₂-C₁₀ heteroaryl, C₁-C₆-alkoxy, C₂-C₆ alkanoyloxy, C₂-C₆ alkanoylamino, C₁-C₆ alkylthio, C₁-C₆ alkylsulfonyl, C₂-C₆ alkoxycarbonyl, CONR₅R₆, O—R₅, NHSO₂R₅ and NHCO₂R₅, wherein the heteroatoms in heteroaryl and heterocycloalkyl are selected from the group consisting of sulfur, nitrogen, and oxygen.

R₅ and R₆ are independently selected from hydrogen, C₁-C₆ alkyl, C₃-C₆ alkenyl, C₅-C₆ aryl, optionally substituted C₅-C₁₀ aryl, optionally substituted C₂-C₁₀ heteroaryl, wherein optional substitution is with one to three groups selected from halogen, C₁-C₆ alkyl, C₁-C₆ alkoxy, C₅-C₆ aryl, and C₃-C₆ heteroaryl, wherein the heteroatom(s) in heteroaryl is/are independently selected from the group consisting of sulfur, nitrogen, and oxygen.

Het is selected from any heterocycle or heteroaryl, optionally substituted with from 0 to 4 substituents independently chosen from: (i) C₁-C₆ alkyl, C₁-C₆ alkoxy, C₂-C₆ alkenyl, C₂-C₆ alkynyl; (ii) halogen, hydroxy, amino, amide, cyano, —COOH, —SO₂NH₂, oxo, nitro and alkoxycarbonyl; (iii) C₅-C₆ aryl; and (iv) C₂-C₆ heteroaryl, wherein the heteroatom(s) in Het is/are independently selected from the group consisting of sulfur, nitrogen, and oxygen.

In still another contemplated aspect, compounds according to the inventive subject matter will have a structure according to Formula (Ie):

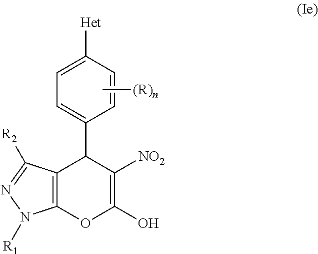

and pharmaceutically acceptable enantiomers, tautomers, diastereomers, racemates, and salts thereof, wherein:

R is independently selected from the group consisting of hydrogen, halogen, hydroxy, amino, cyano, —COOH, —SO₂NH₂, oxo, nitro, C₁-C₆ alkyl, C₂-C₆ alkenyl, C₂-C₆ alkynyl, C₃-C₆ cyclalkyl, C₅-C₆ aryl, substituted C₅-C₆ aryl, C₃-C₆ heteroaryl, substituted C₃-C₆ heteroaryl, C₂-C₆ alkoxycarbonyl, CONHSO₂R₅ and CONR₅R₆, O—R₅, S—R₅, SO—R₅, SO₂—R₅, NHSO₂R₅ and NHCO₂R₅, and wherein n is an integer between 0 and 4.

R₁ is selected from hydrogen, C₁-C₆ alkyl, C₃-C₆ alkenyl, C₅-C₆ aryl, substituted C₅-C₆ aryl, C₅-C₆ heteroaryl, substituted C₅-C₆ heteroaryl, C₅-C₁₀ alkylaryl.

R₂ is selected from hydrogen, halogen, amino, CN, COOH, C₁-C₁₀ alkyl, C₁-C₁₀ cycloalkyl, C₂-C₁₀ alkenyl, C₅-C₁₀ aryl, C₅-C₁₀ arylalkyl, substituted C₅-C₆ aryl, C₂-C₁₀ optionally substituted heteroaryl, optionally substituted heterocycloalkyl, optionally substituted heterocycloalkyl fused to aryl, substituted C₂-C₁₀ heteroaryl, C₁-C₆-alkoxy, C₂-C₆ alkanoyloxy, C₂-C₆ alkanoylamino, C₁-C₆ alkylthio, C₁-C₆ alkylsulfonyl, C₂-C₆ alkoxycarbonyl, CONR₅R₆, O—R₅, NHSO₂R₅ and NHCO₂R₅, wherein the heteroatoms in heteroaryl and heterocycloalkyl are selected from the group consisting of sulfur, nitrogen, and oxygen.

R₅ and R₆ are independently selected from hydrogen, C₁-C₆ alkyl, C₃-C₆ alkenyl, C₅-C₆ aryl, optionally substituted C₅-C₁₀ aryl, optionally substituted C₂-C₁₀ heteroaryl, wherein optional substitution is with one to three groups selected from halogen, C₁-C₆ alkyl, C₁-C₆ alkoxy, C₅-C₆ aryl, and C₃-C₆ heteroaryl, wherein the heteroatom(s) in heteroaryl is/are independently selected from the group consisting of sulfur, nitrogen, and oxygen.

Het is selected from any heterocycle or heteroaryl, optionally substituted with from 0 to 4 substituents independently chosen from: (i) C₁-C₆ alkyl, C₁-C₆ alkoxy, C₂-C₆ alkenyl, C₂-C₆ alkynyl; (ii) halogen, hydroxy, amino, amide, cyano, —COOH, —SO₂NH₂, oxo, nitro and alkoxycarbonyl; (iii) C₅-C₆ aryl; and (iv) C₂-C₆ heteroaryl, wherein the heteroatom(s) in Het is/are independently selected from the group consisting of sulfur, nitrogen, and oxygen.

In a further contemplated aspect, compounds according to the inventive subject matter will have a structure according to Formula (If):

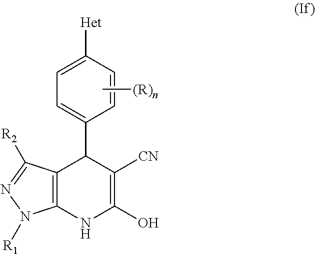

and pharmaceutically acceptable enantiomers, tautomers, diastereomers, racemates, and salts thereof, wherein:

R is independently selected from the group consisting of hydrogen, halogen, hydroxy, amino, cyano, —COOH, —SO₂NH₂, oxo, nitro, C₁-C₆ alkyl, C₂-C₆ alkenyl, C₂-C₆ alkynyl, C₃-C₆ cyclalkyl, C₅-C₆ aryl, substituted C₅-C₆ aryl, C₃-C₆ heteroaryl, substituted C₃-C₆ heteroaryl, C₂-C₆ alkoxycarbonyl, CONHSO₂R₅ and CONR₅R₆, O—R₅, S—R₅, SO—R₅, SO₂—R₅, NHSO₂R₅ and NHCO₂R₅, and wherein n is an integer between 0 and 4.

R₁ is selected from hydrogen, C₁-C₆ alkyl, C₃-C₆ alkenyl, C₅-C₆ aryl, substituted C₅-C₆ aryl, C₅-C₆ heteroaryl, substituted C₅-C₆ heteroaryl, C₅-C₁₀ alkylaryl.

R₂ is selected from hydrogen, halogen, amino, CN, COOH, C₁-C₁₀ alkyl, C₁-C₁₀ cycloalkyl, C₂-C₁₀ alkenyl, C₅-C₁₀ aryl, C₅-C₁₀ arylalkyl, substituted C₅-C₆ aryl, C₂-C₁₀ optionally substituted heteroaryl, optionally substituted heterocycloalkyl, optionally substituted heterocycloalkyl fused to aryl, substituted C₂-C₁₀ heteroaryl, C₁-C₆-alkoxy, C₂-C₆ alkanoyloxy, C₂-C₆ alkanoylamino, C alkylthio, C₁-C₆ alkylsulfonyl, C₂-C₆ alkoxycarbonyl, CONR₅R₆, O—R₅, NHSO₂R₅ and NHCO₂R₅, wherein the heteroatoms in heteroaryl and heterocycloalkyl are selected from the group consisting of sulfur, nitrogen, and oxygen.

R₅ and R₆ are independently selected from hydrogen, C₁-C₆ alkyl, C₃-C₆ alkenyl, C₅-C₆ aryl, optionally substituted C₅-C₁₀ aryl, optionally substituted C₂-C₁₀ heteroaryl, wherein optional substitution is with one to three groups selected from halogen, C₁-C₆ alkyl, C₁-C₆ alkoxy, C₅-C₆ aryl, and C₃-C₆ heteroaryl, wherein the heteroatom(s) in heteroaryl is/are independently selected from the group consisting of sulfur, nitrogen, and oxygen.

Het is selected from any heterocycle or heteroaryl, optionally substituted with from 0 to 4 substituents independently chosen from: (i) C₁-C₆ alkyl, C alkoxy, C₂-C₆ alkenyl, C₂-C₆ alkynyl; (ii) halogen, hydroxy, amino, amide, cyano, —COOH, —SO₂NH₂, oxo, nitro and alkoxycarbonyl; (iii) C₅-C₆ aryl; and (iv) C₂-C₆ heteroaryl, wherein the heteroatom(s) in Het is/are independently selected from the group consisting of sulfur, nitrogen, and oxygen.

Most typically, Het is selected from the group of:

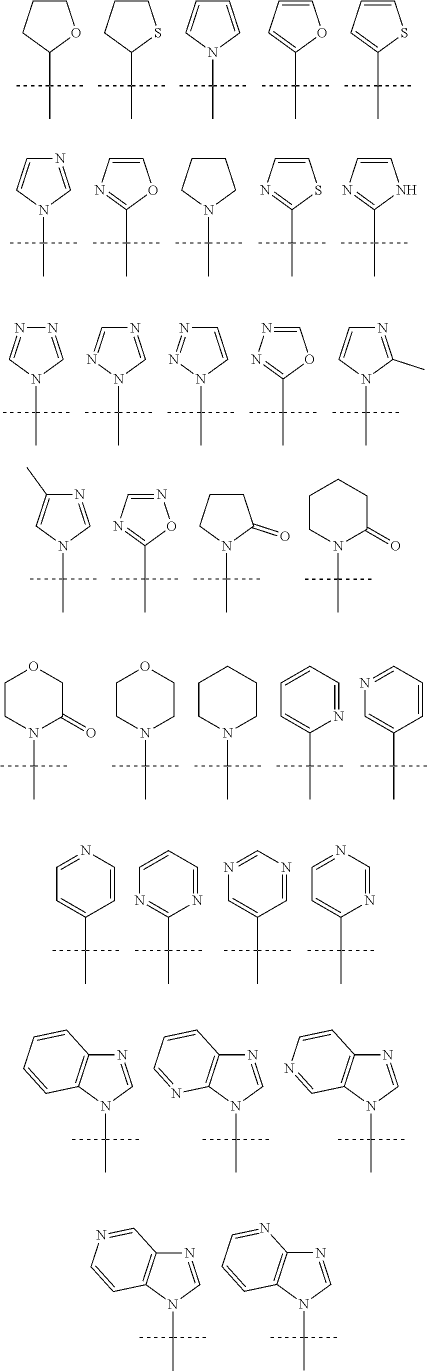

In still further contemplated aspects, R₂ is selected from: (i) Hydrogen; (ii) C₁-C₆ alkyl, C₂-C₆ alkenyl, C₂-C₆ alkynyl; (iii) arylalkyl, which may have 1-4 optional substituents; and (iv) heterocyclic and heteroaryl:

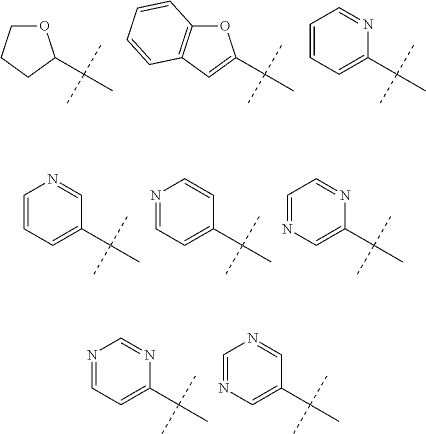

Consequently, contemplated exemplary compounds according to the inventive subject matter include:

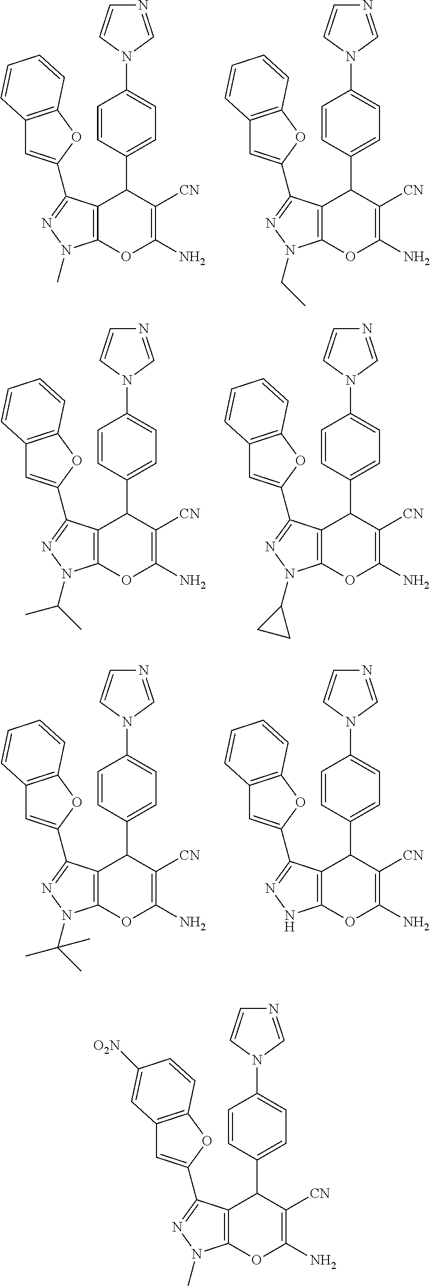

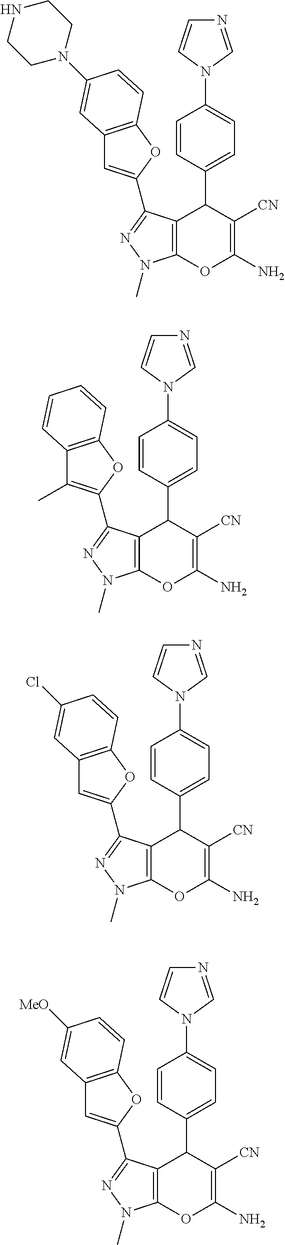

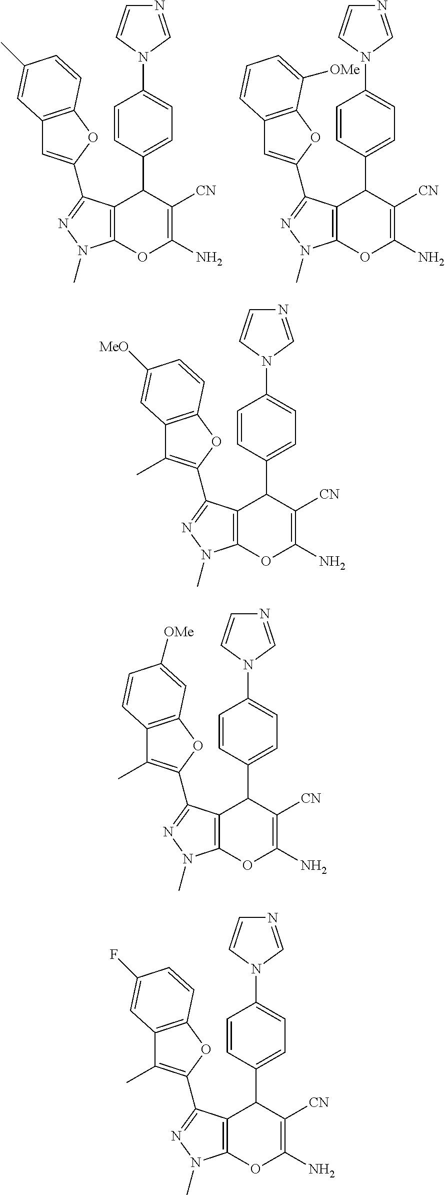

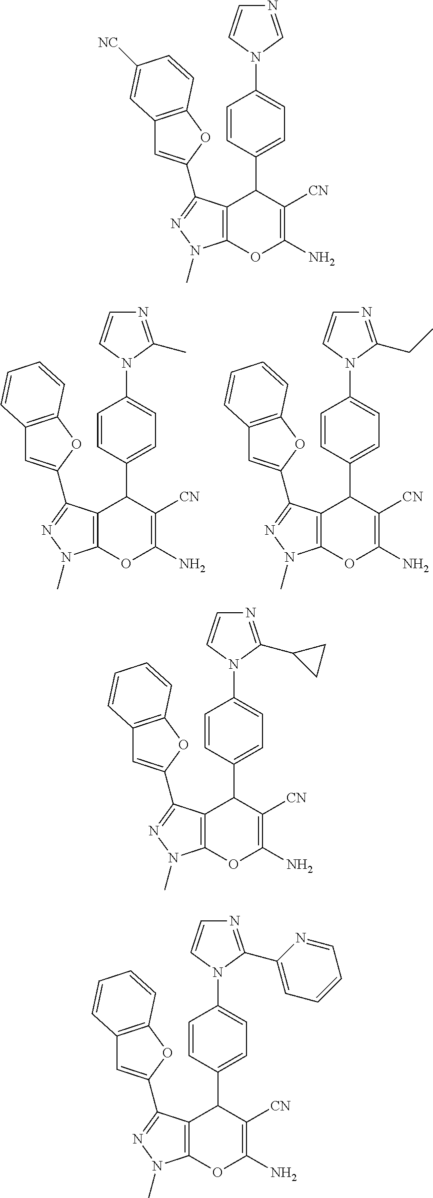

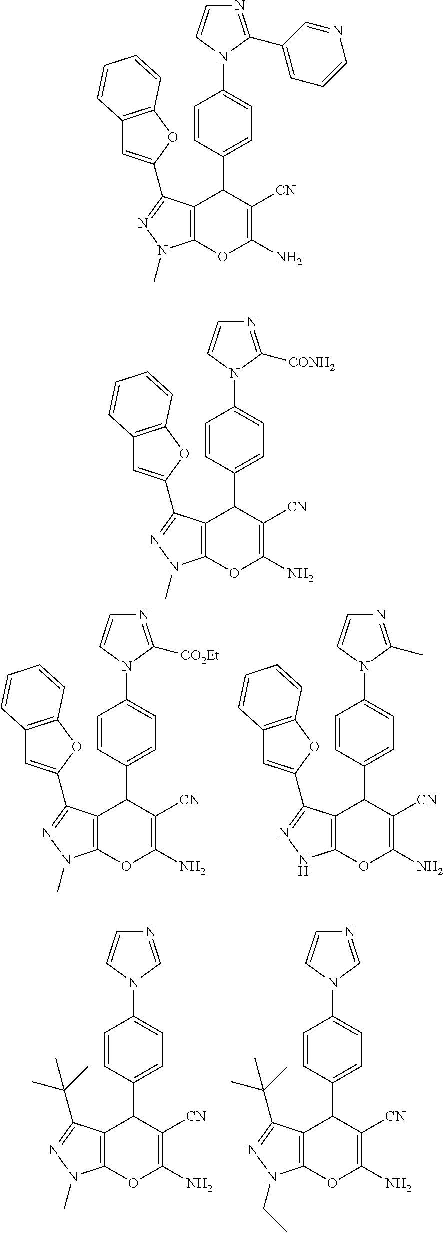

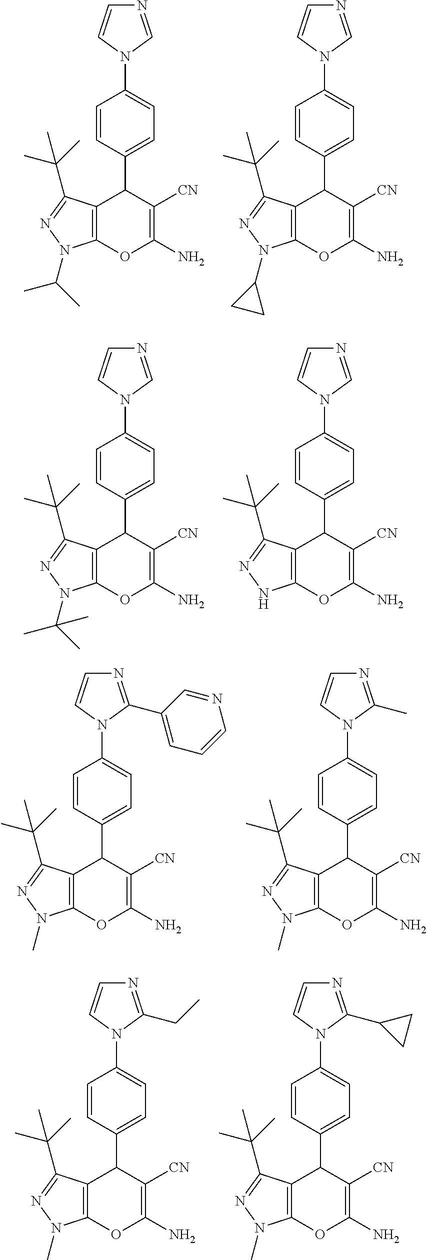

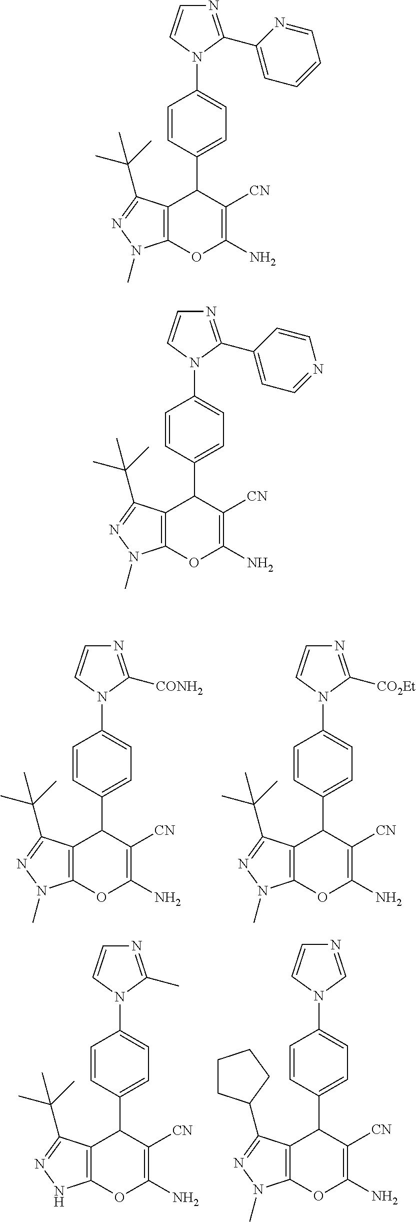 [SMILES: Cn1nc(C(C)(C)C)c2c1OC(N)=C(C#N)C2c1ccc(-n2ccnc2-c2ccccn2)cc1 ; Cn1nc(C(C)(C)C)c2c1OC(N)=C(C#N)C2c1ccc(-n2ccnc2-c2ccncc2)cc1 ; Cn1nc(C(C)(C)C)c2c1OC(N)=C(C#N)C2c1ccc(-n2ccnc2C(N)=O)cc1 ; CCOC(=O)c1nccn1-c1ccc(C2C(C#N)=C(N)Oc3c2c(C(C)(C)C)nn3C)cc1 ; Cc1nccn1-c1ccc(C2C(C#N)=C(N)Oc3[nH]nc(C(C)(C)C)c32)cc1 ; Cn1nc(C2CCCC2)c2c1OC(N)=C(C#N)C2c1ccc(-n2ccnc2)cc1]

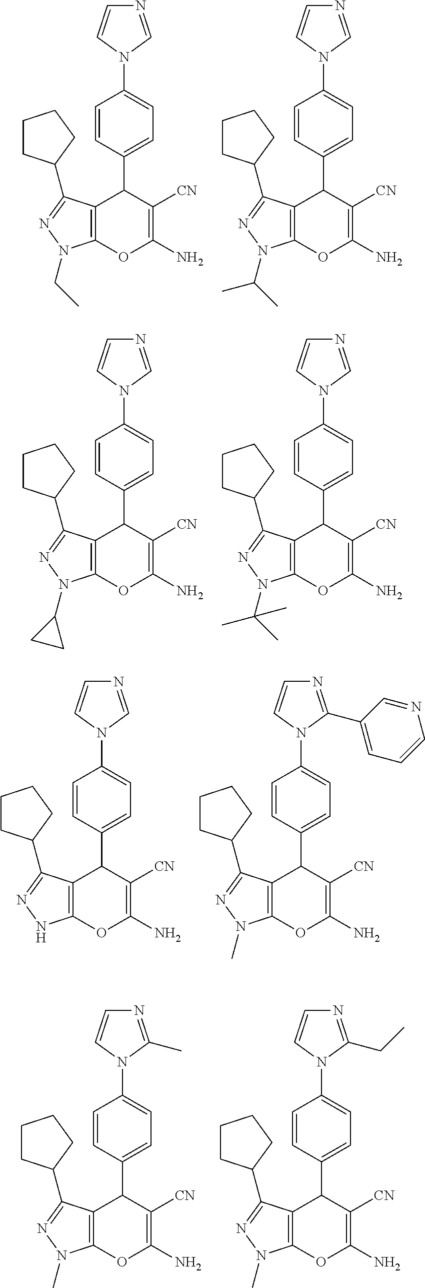

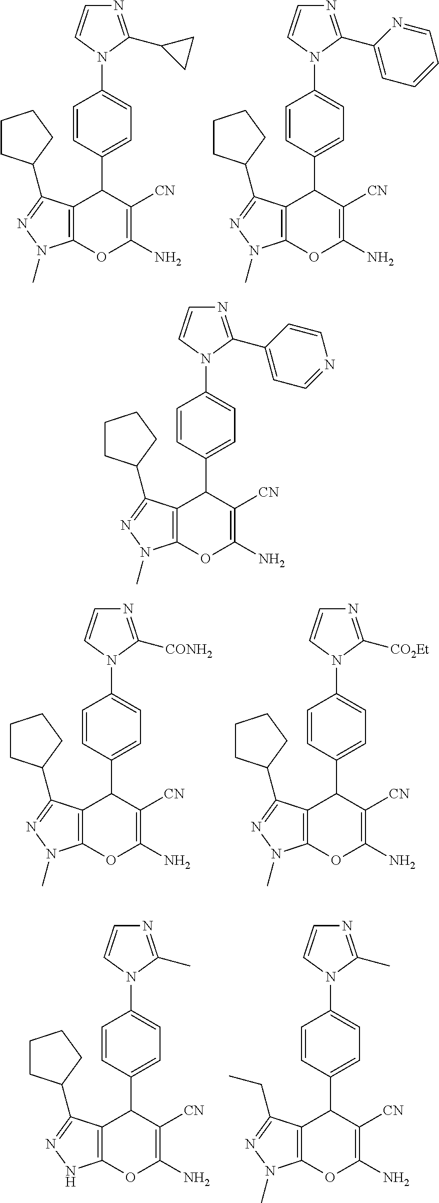

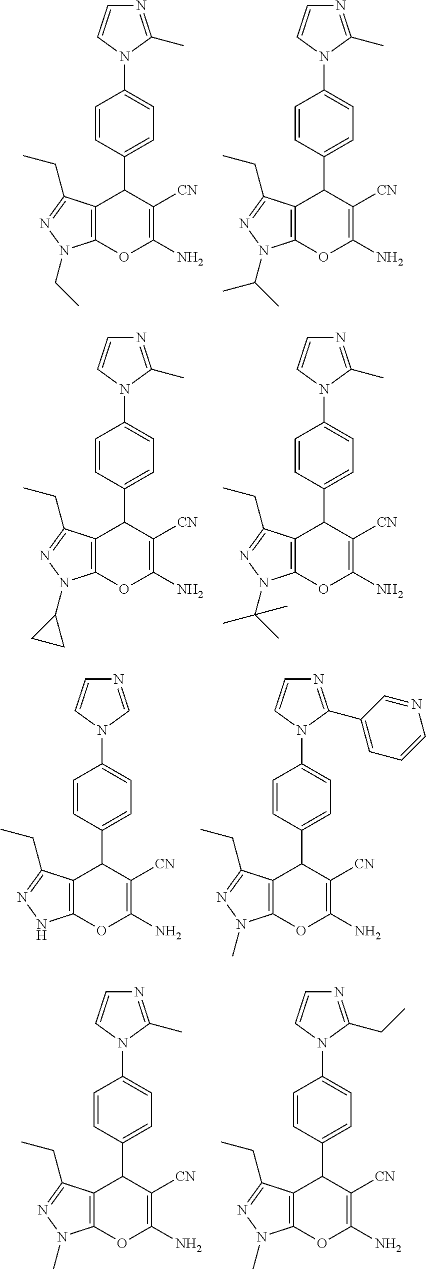

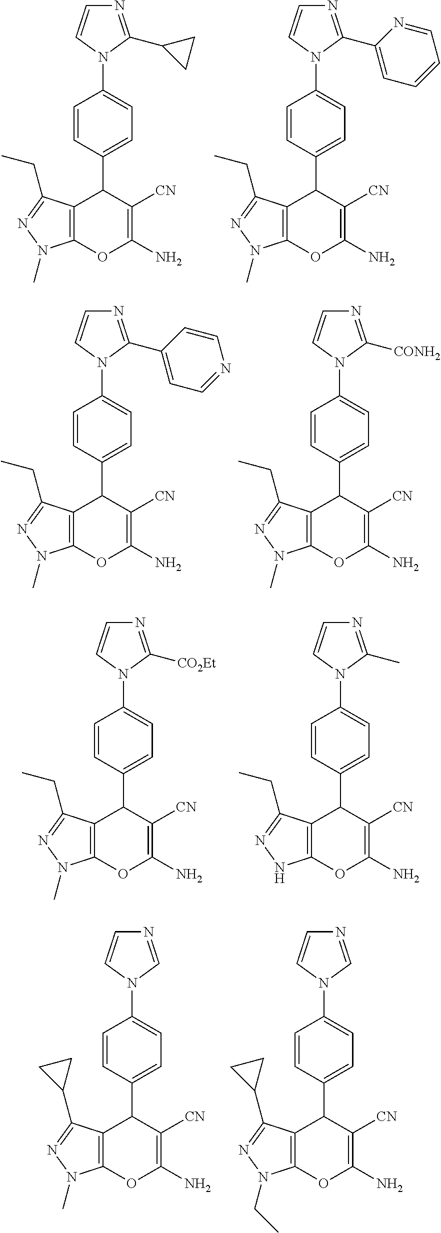

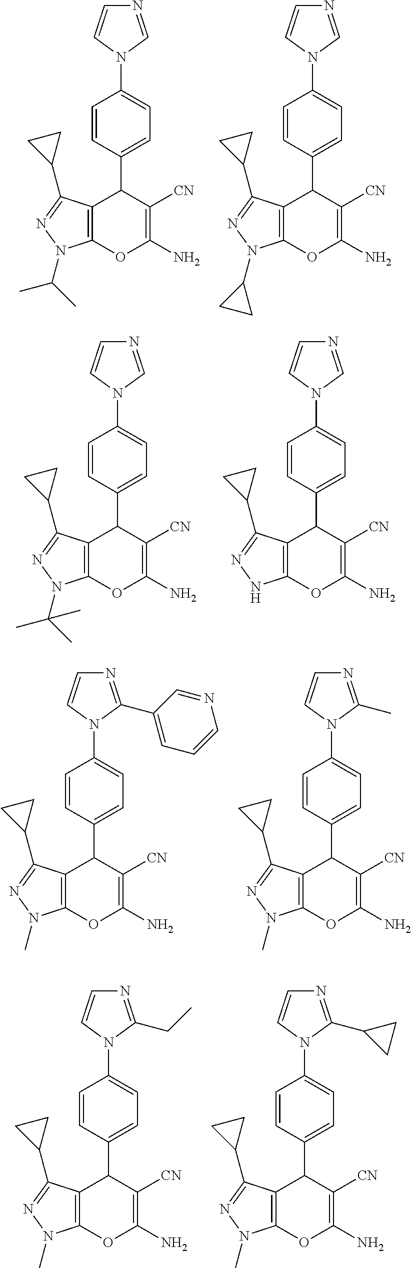

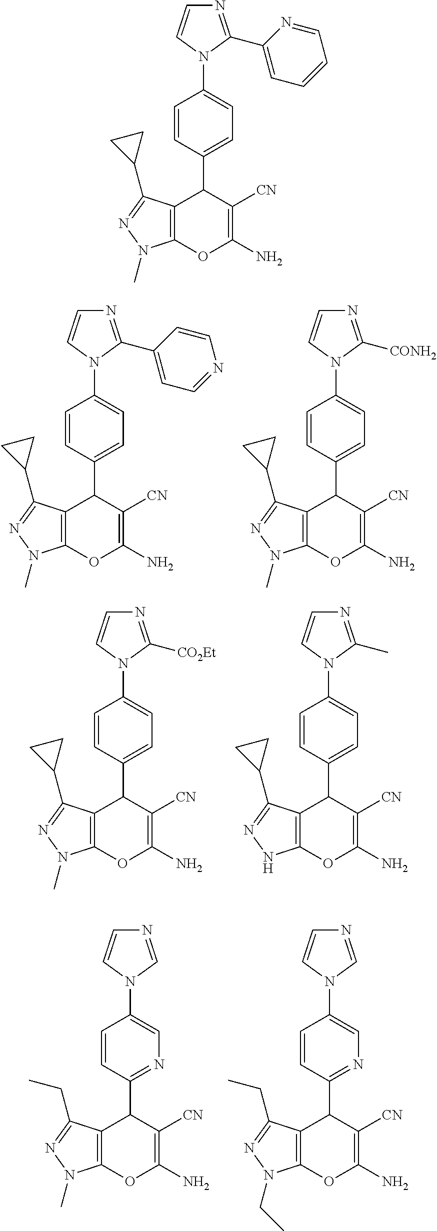

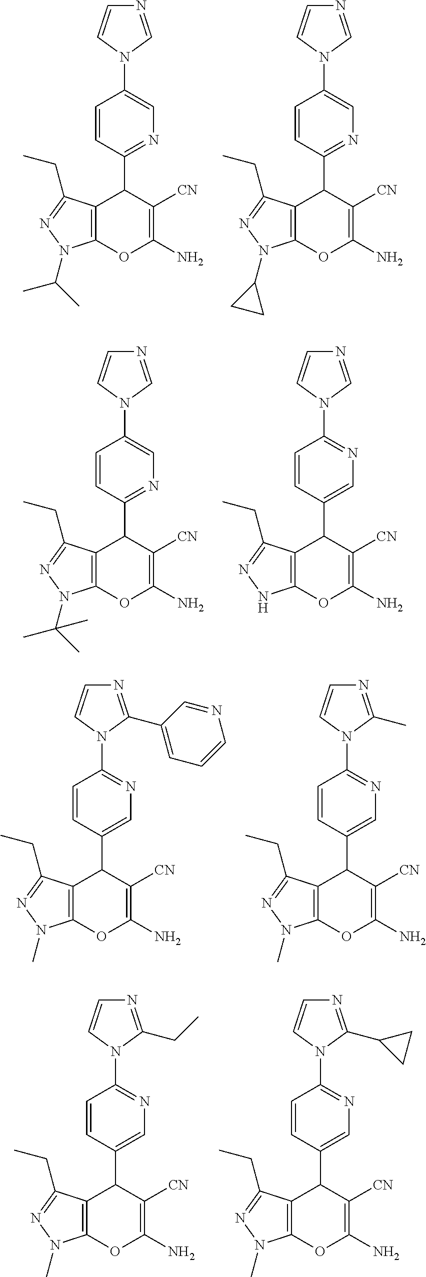

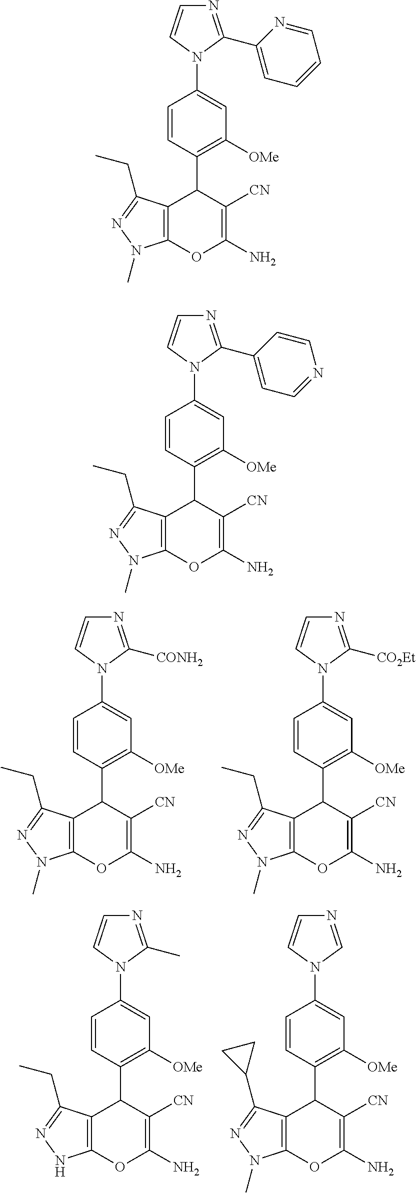

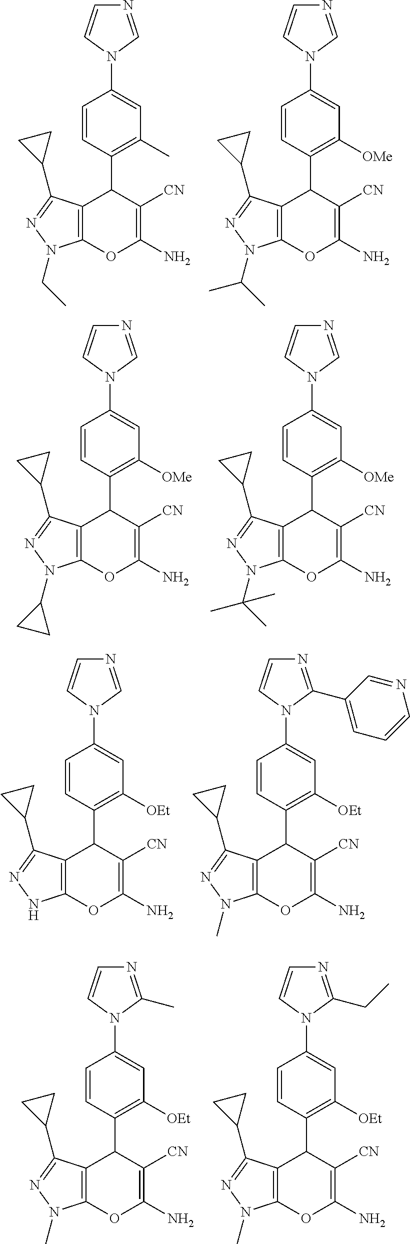

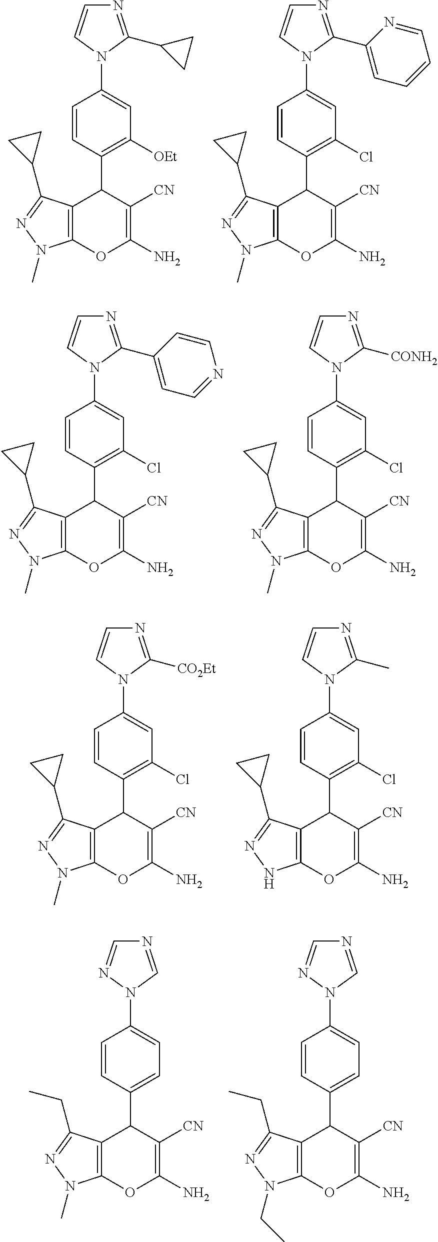

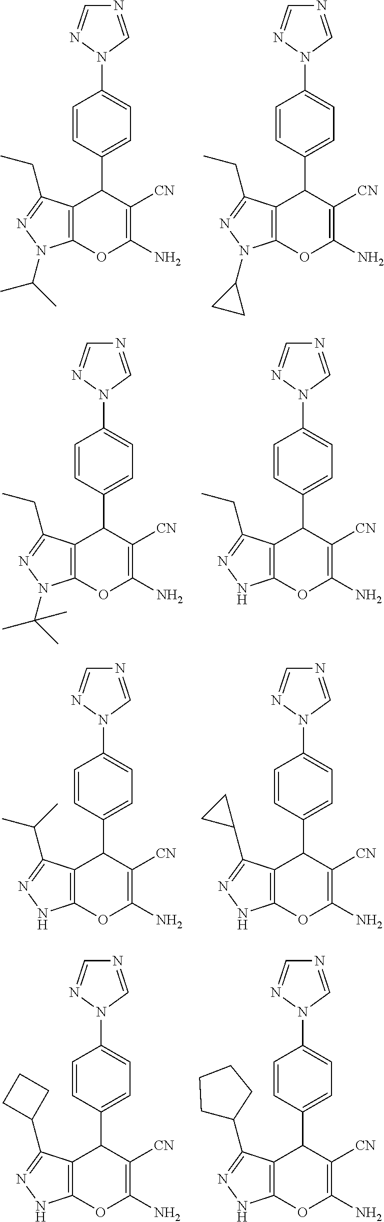

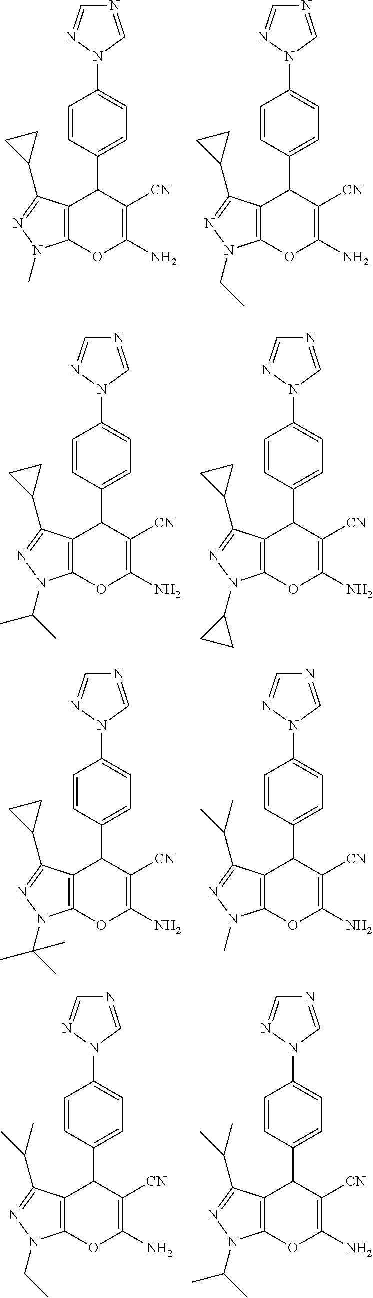

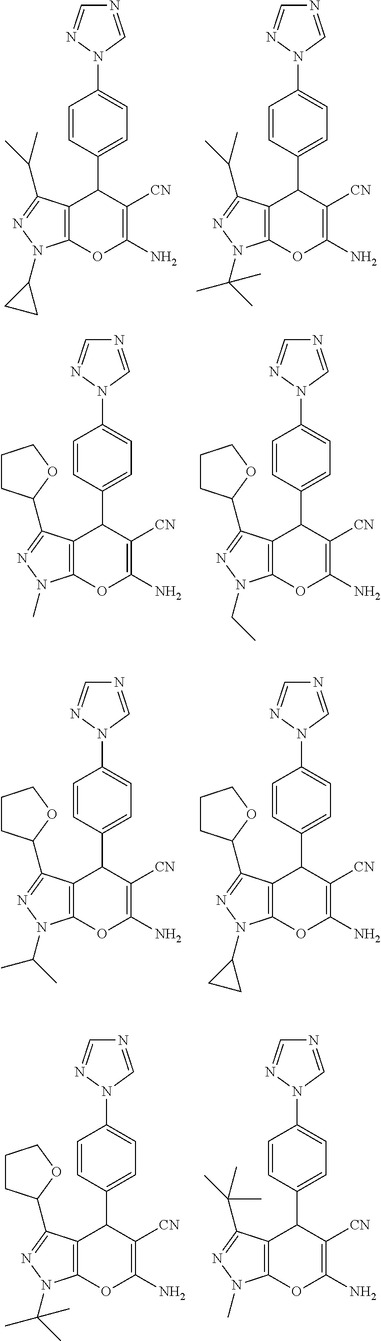

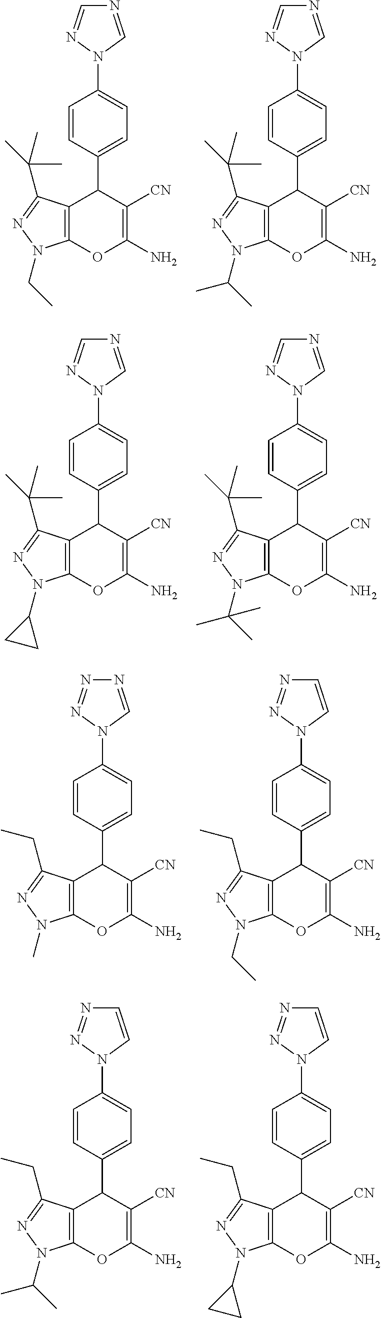

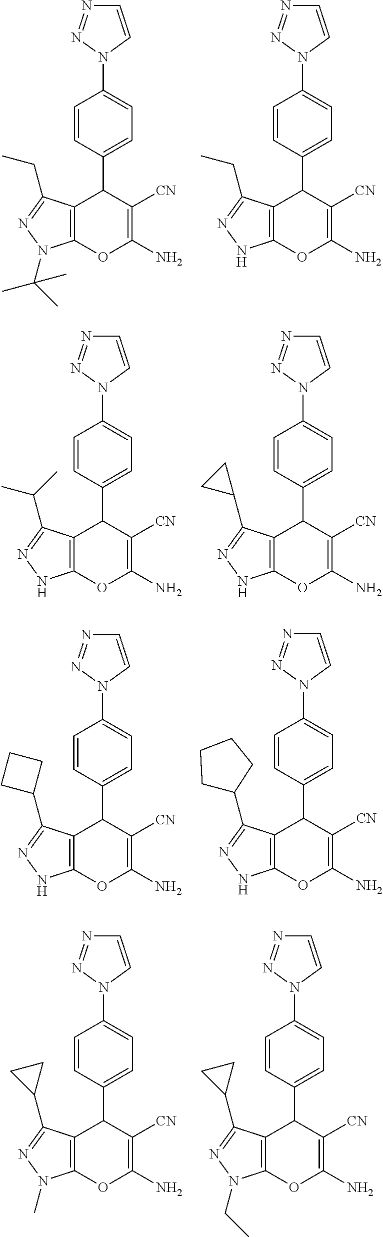

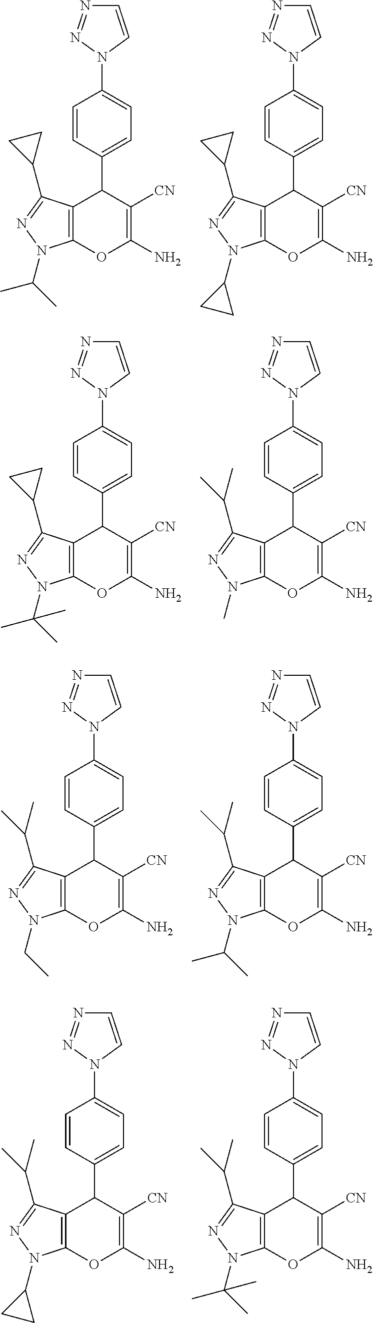

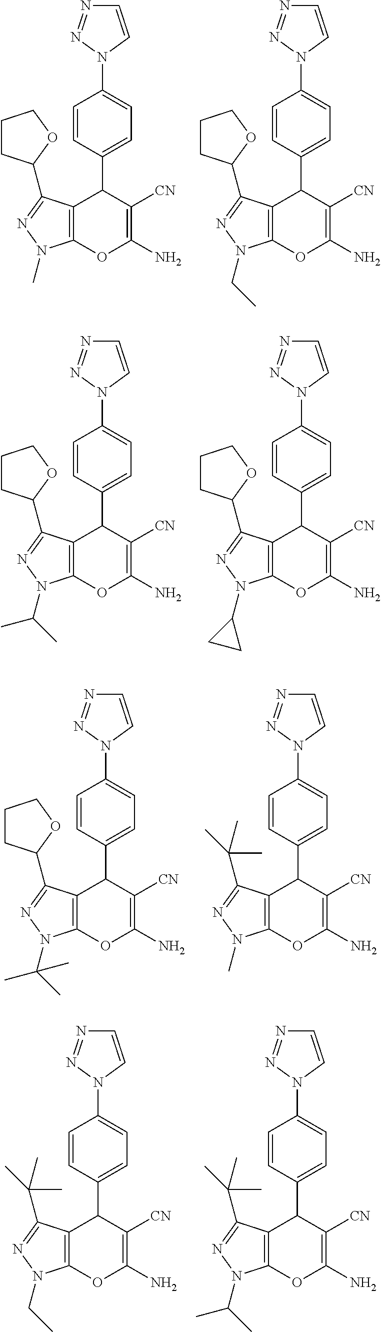

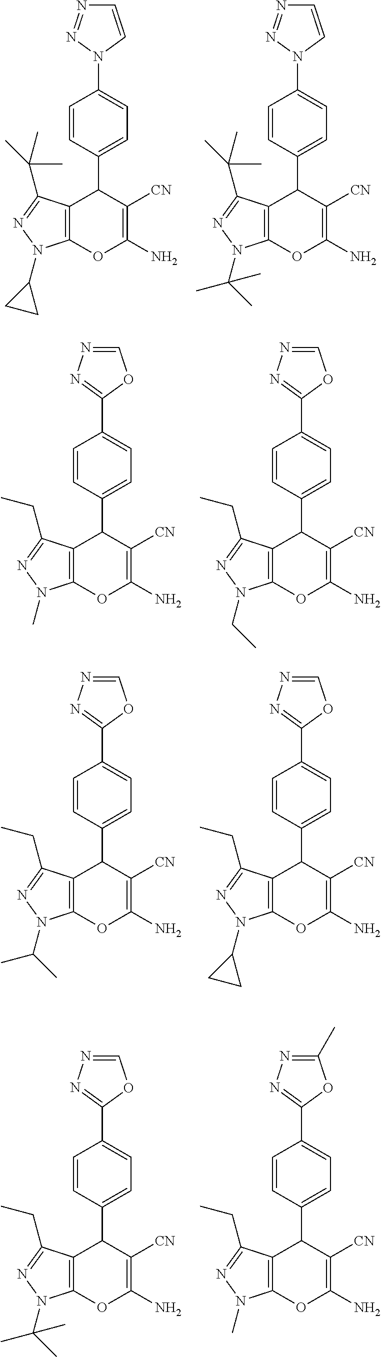

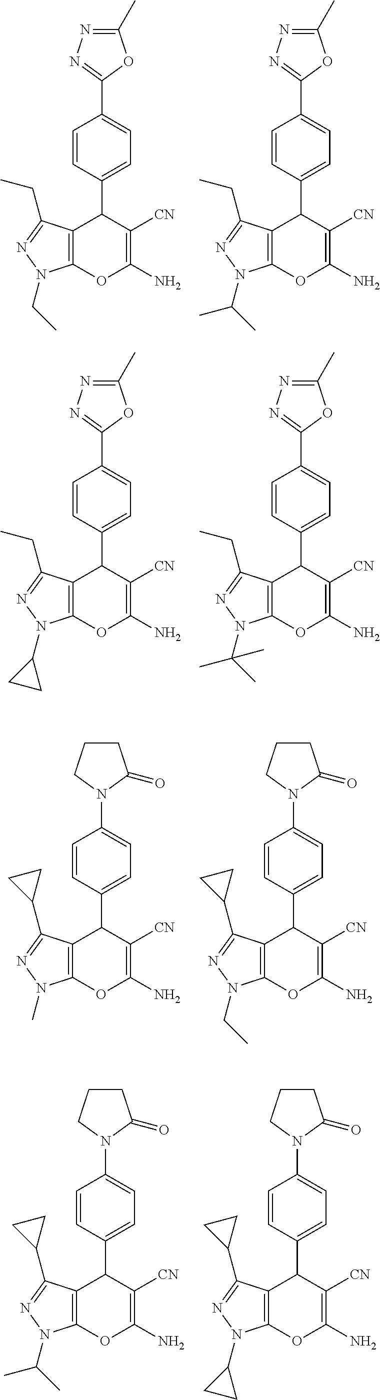

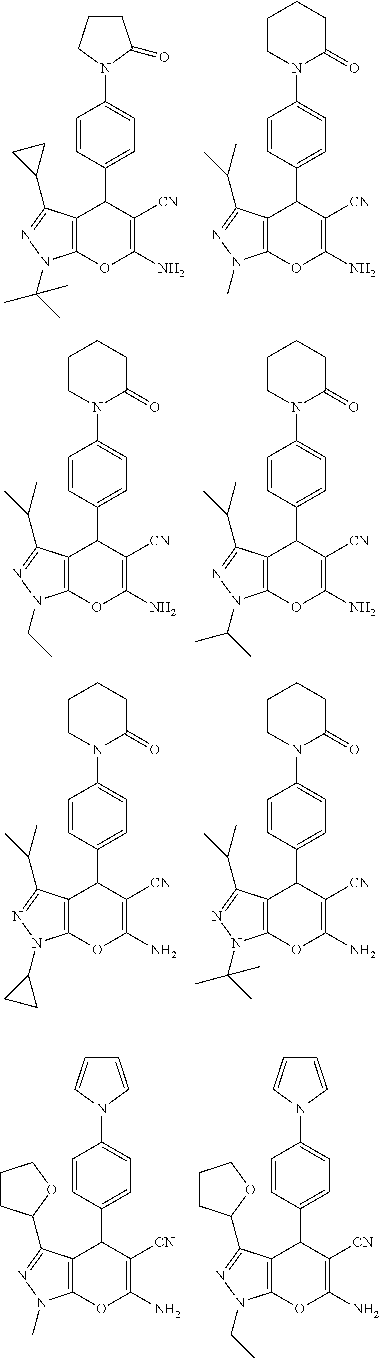

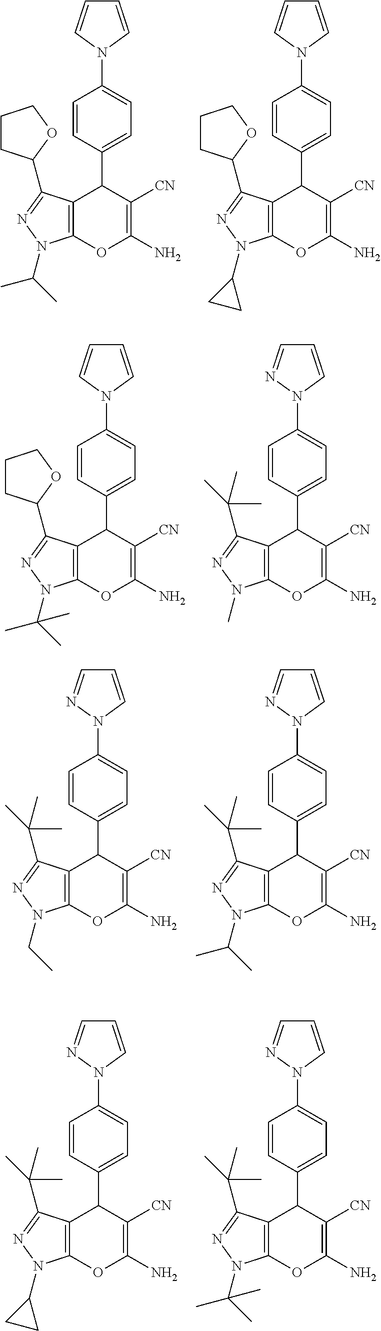

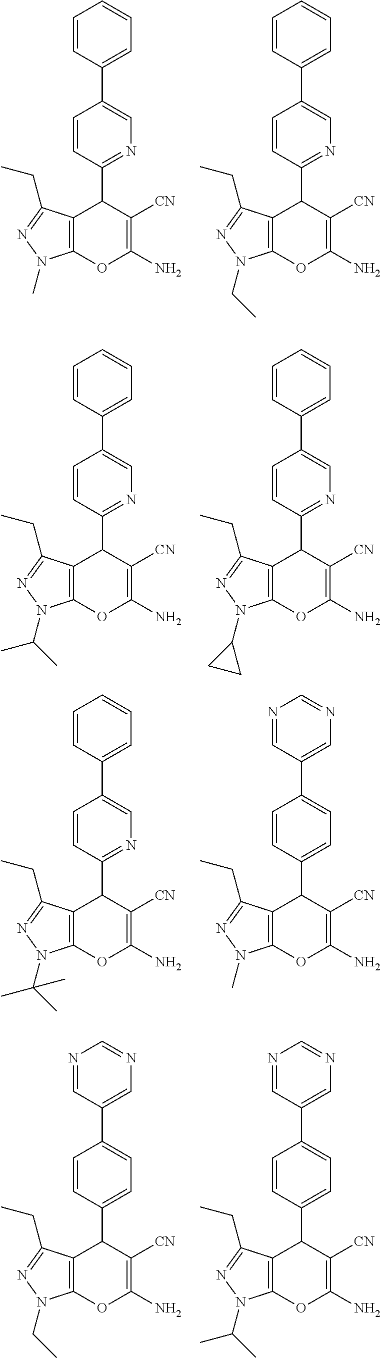

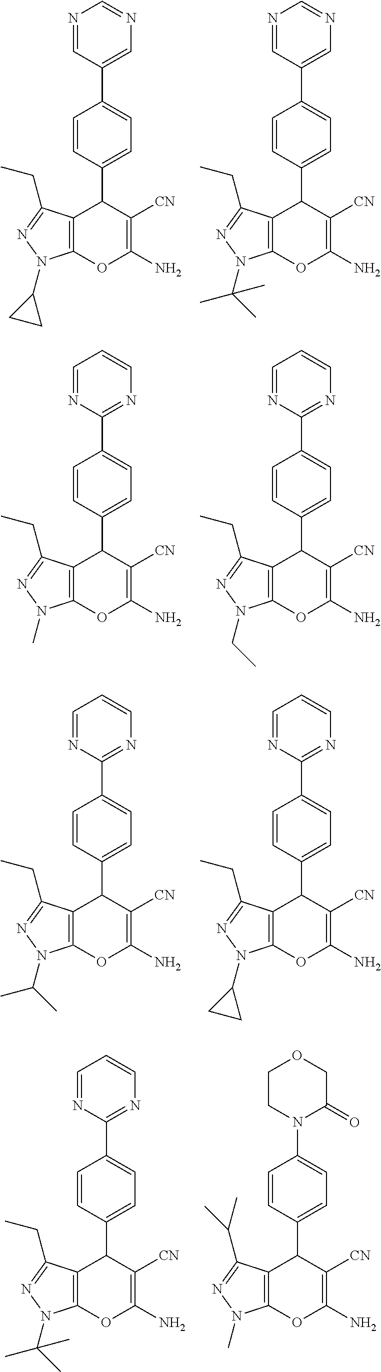

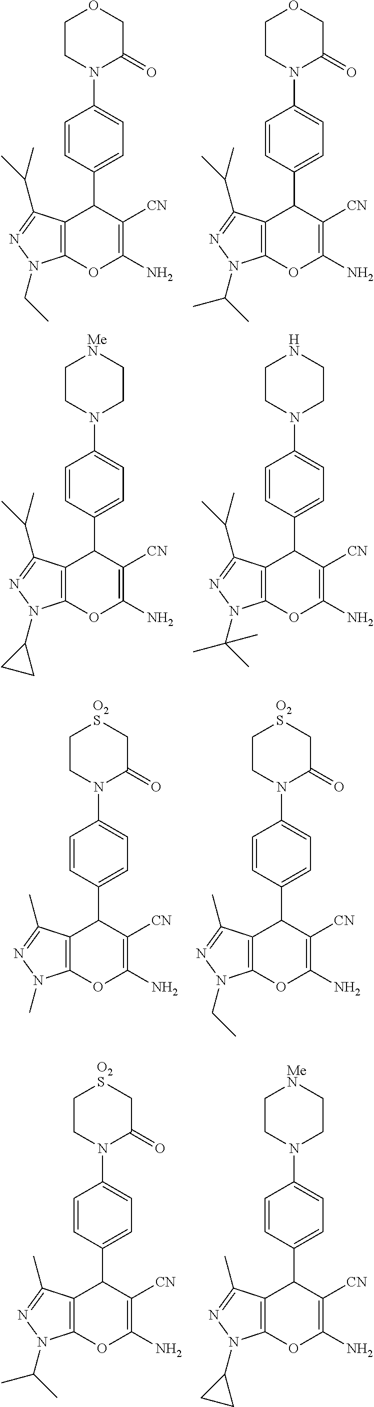

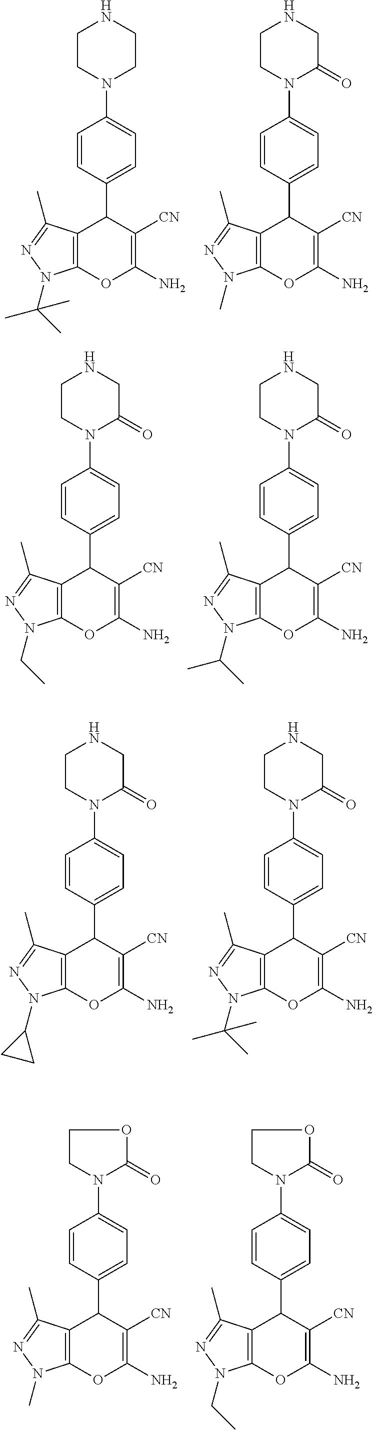

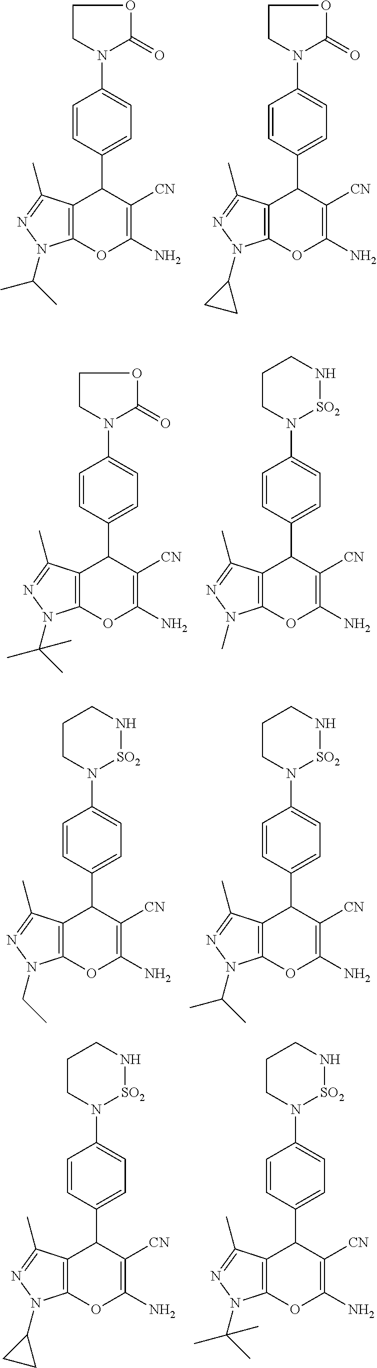

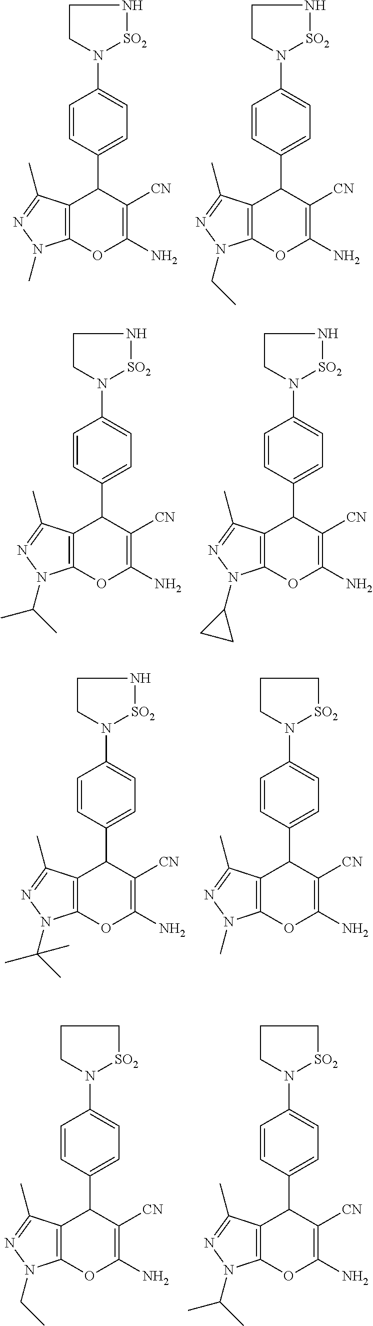

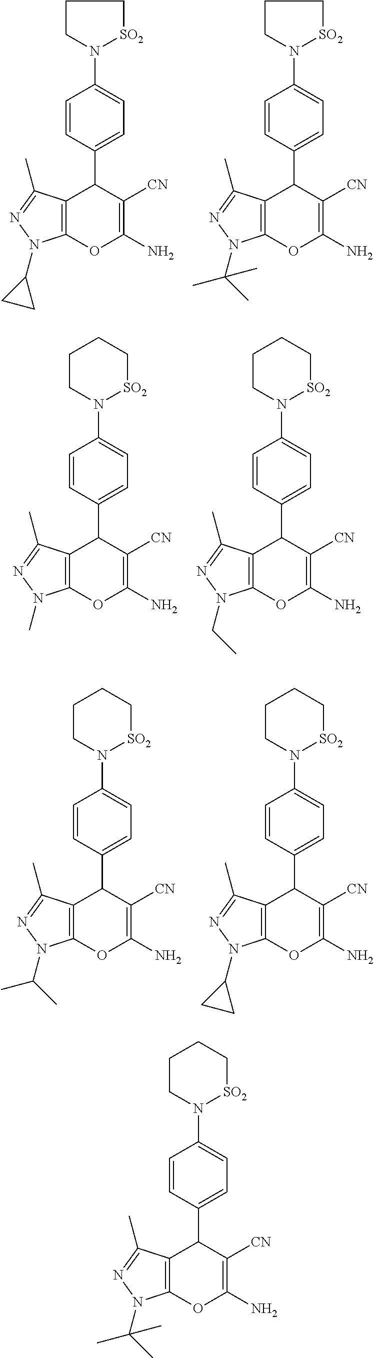

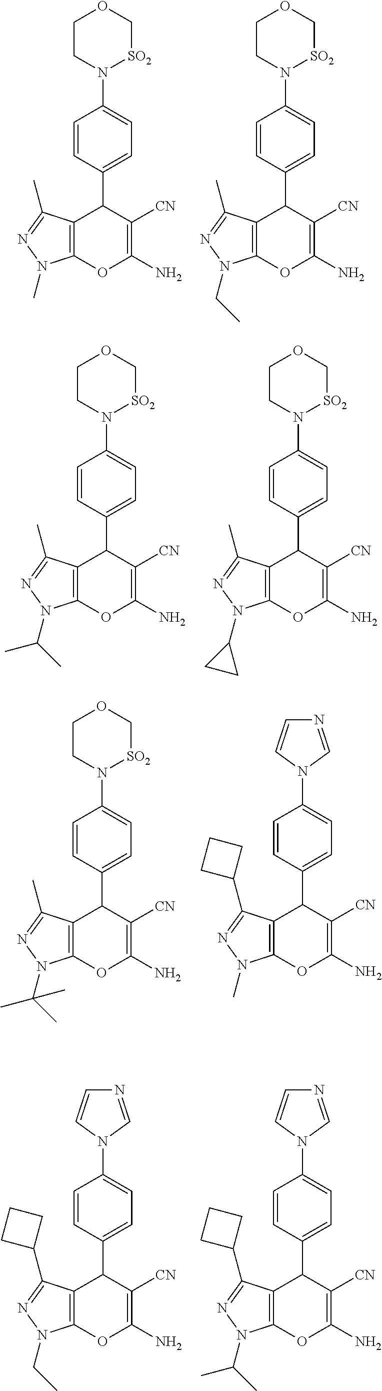

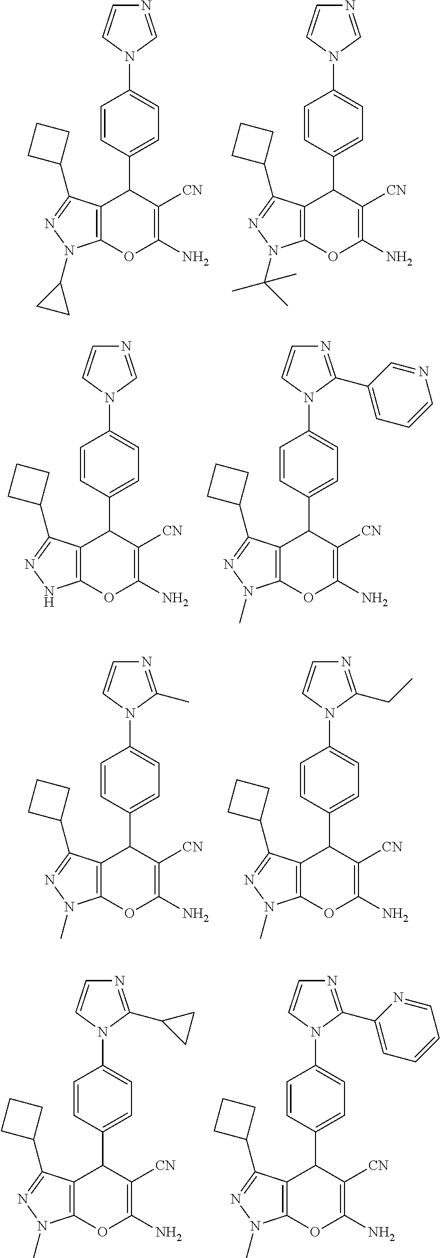

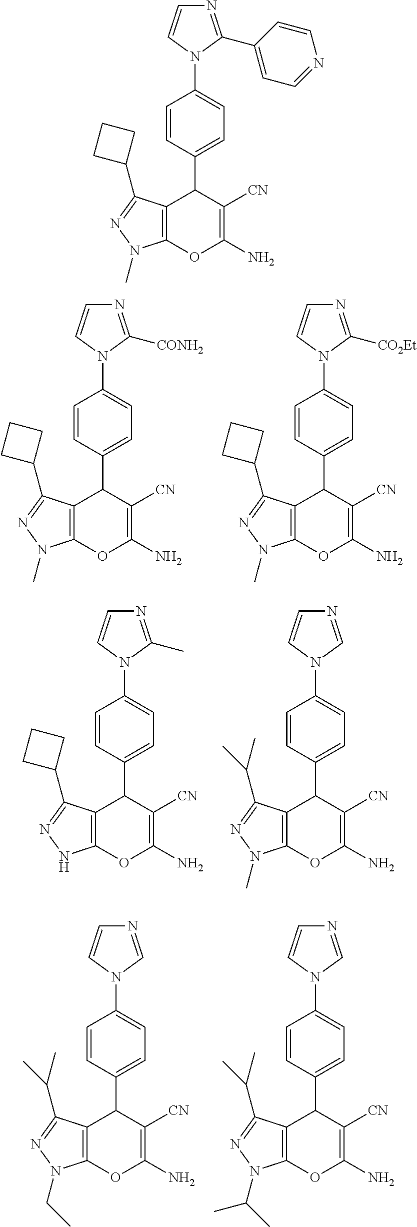

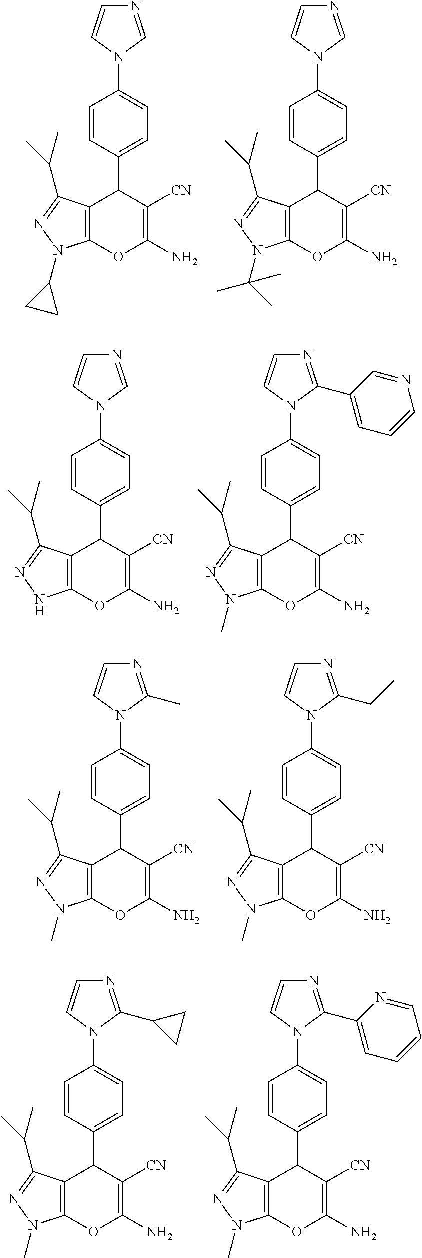

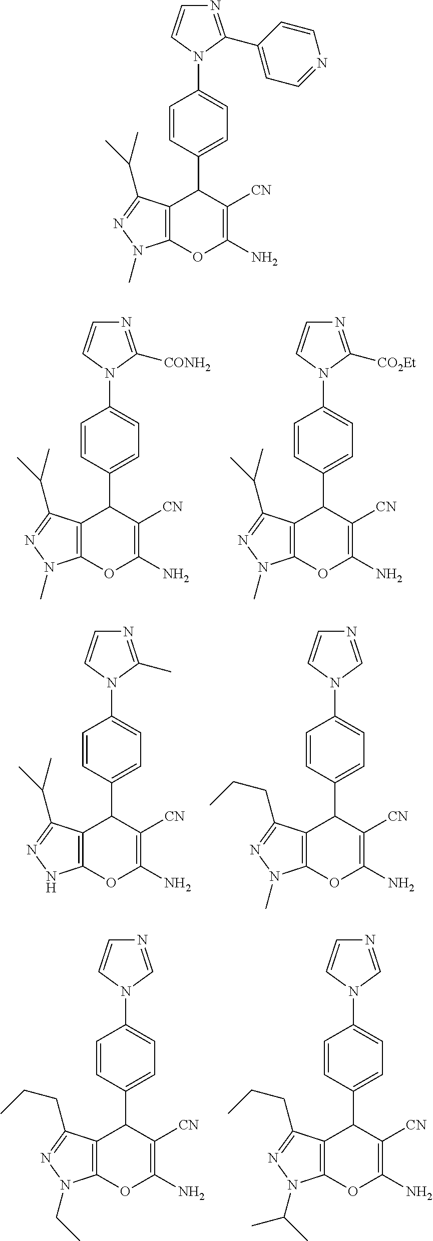

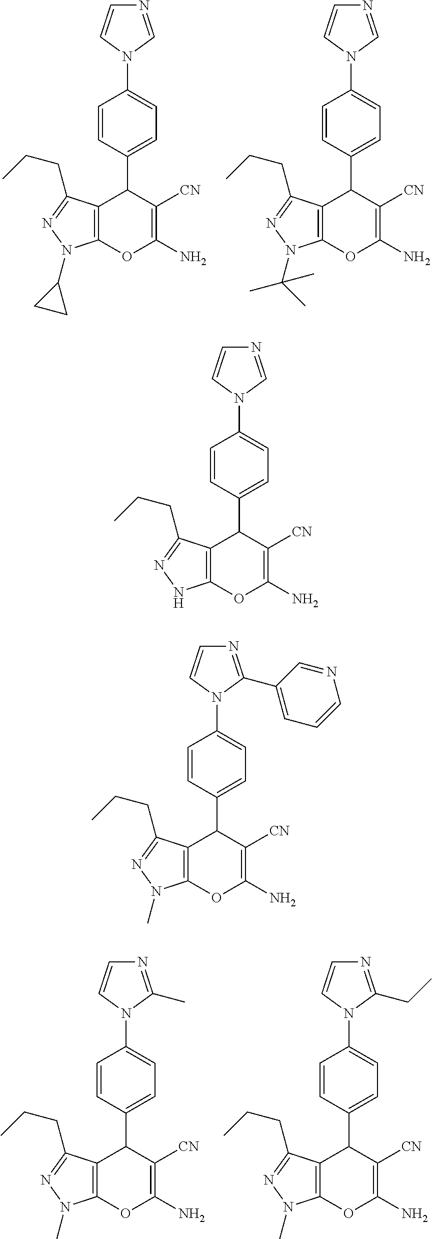

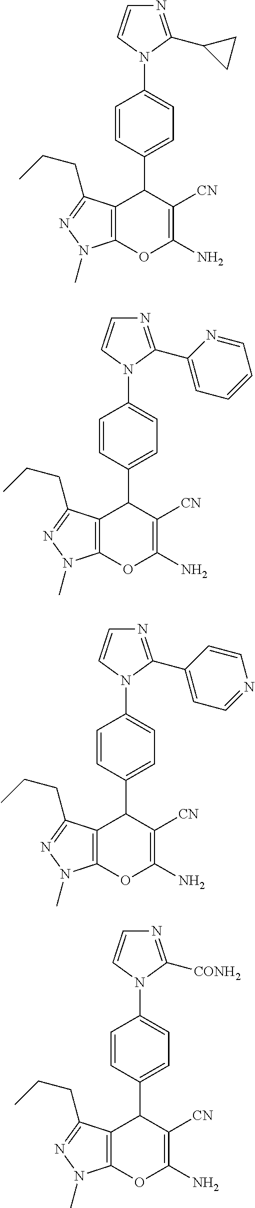 [SMILES: CCCc1nn(C)c2c1C(c1ccc(-n3ccnc3C3CC3)cc1)C(C#N)=C(N)O2 ; CCCc1nn(C)c2c1C(c1ccc(-n3ccnc3-c3ccccn3)cc1)C(C#N)=C(N)O2 ; CCCc1nn(C)c2c1C(c1ccc(-n3ccnc3-c3ccncc3)cc1)C(C#N)=C(N)O2 ; CCCc1nn(C)c2c1C(c1ccc(-n3ccnc3C(N)=O)cc1)C(C#N)=C(N)O2]

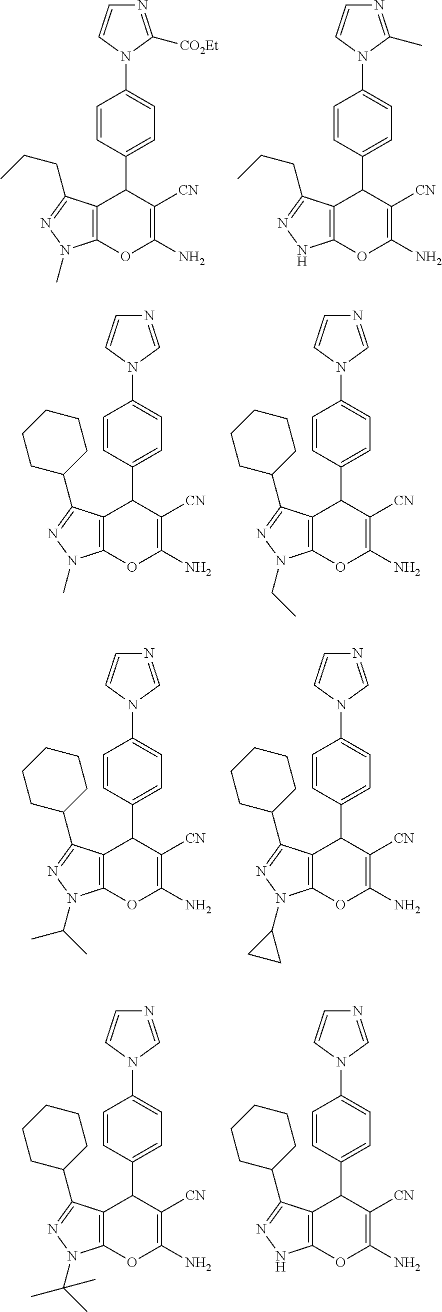

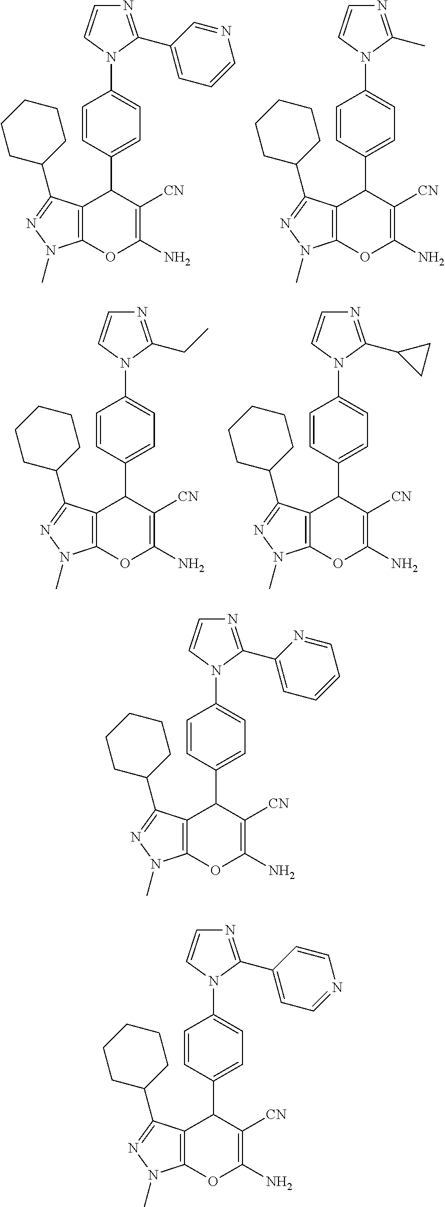

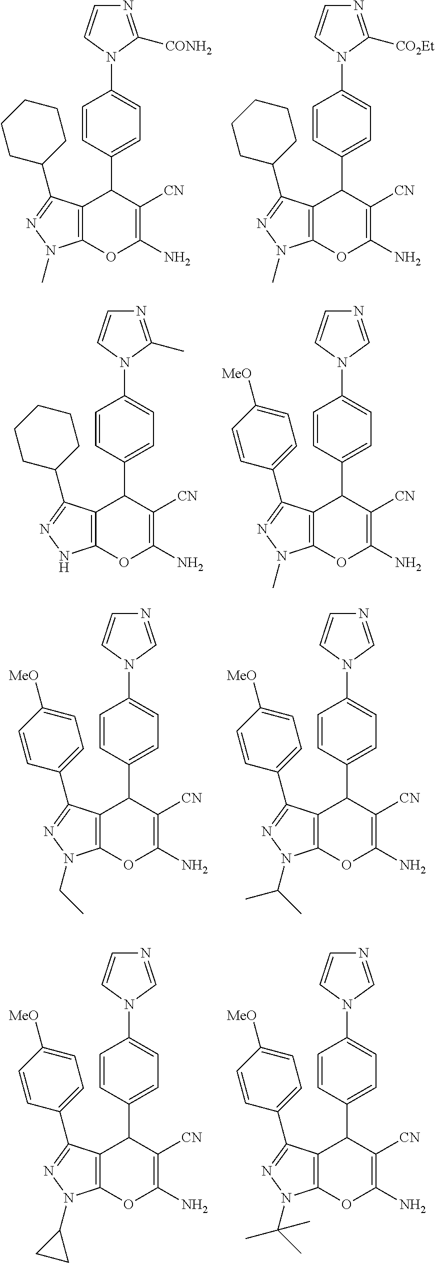

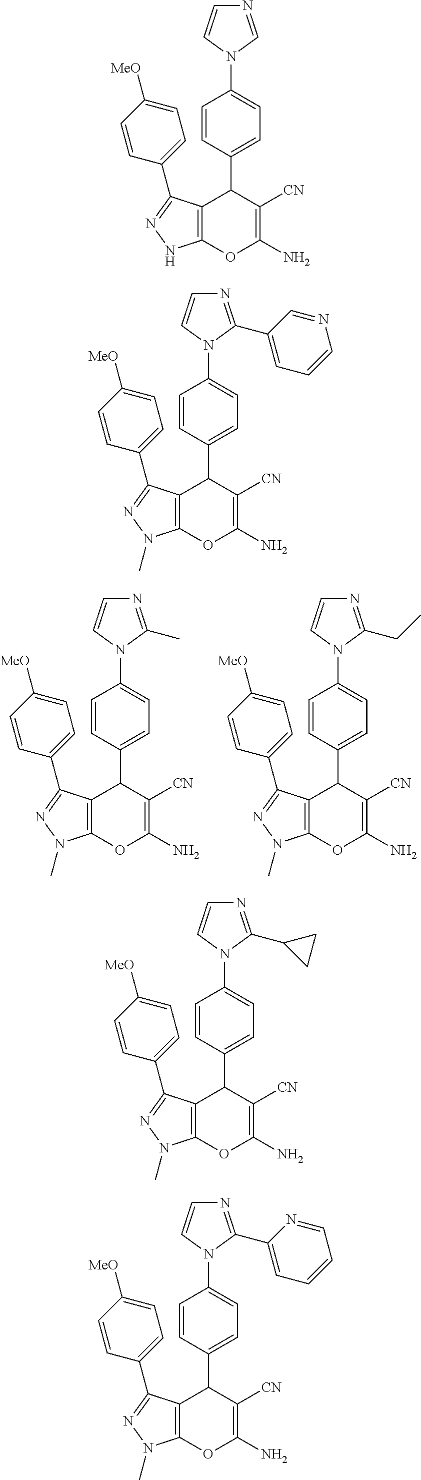

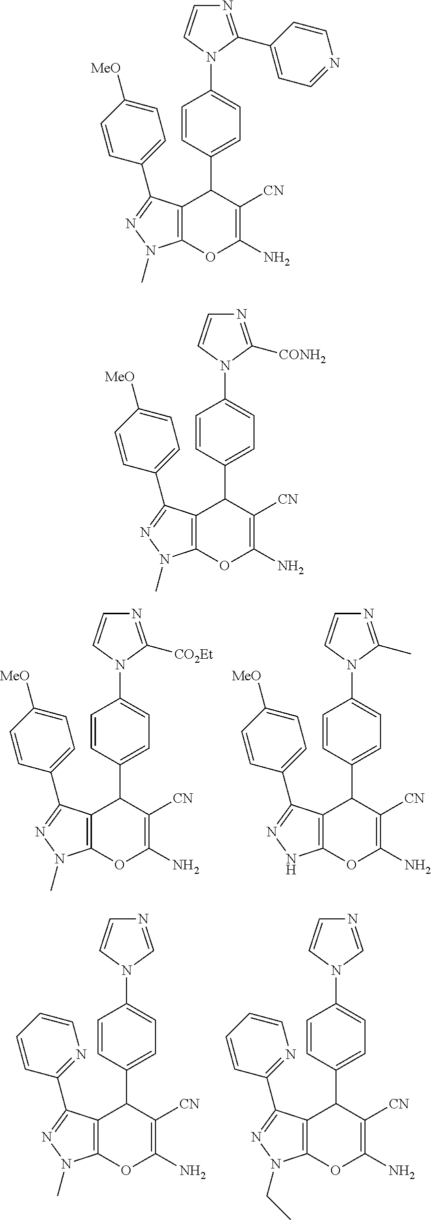

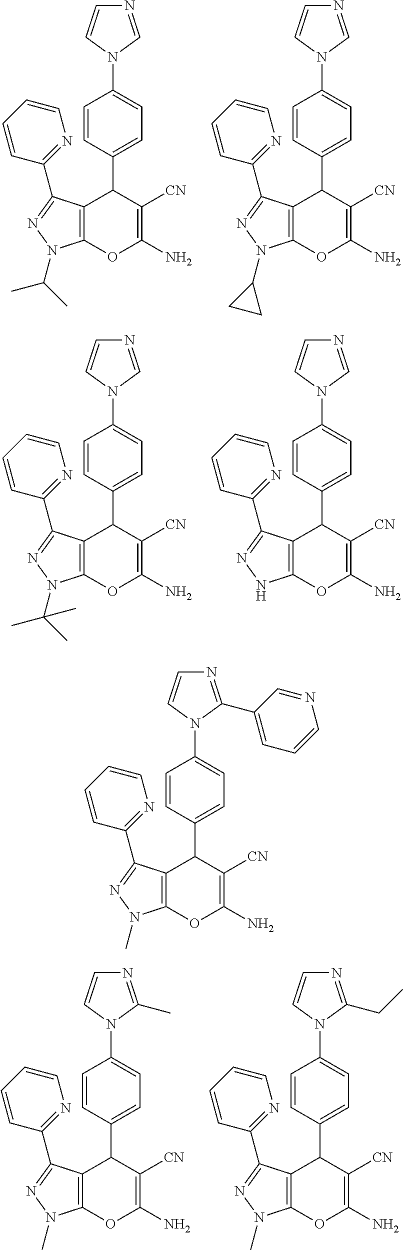

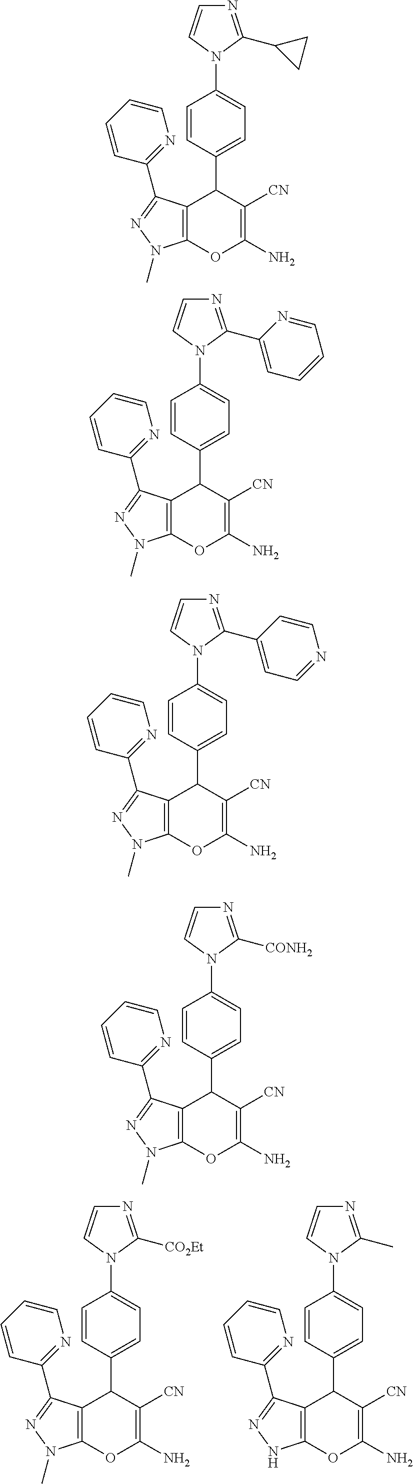

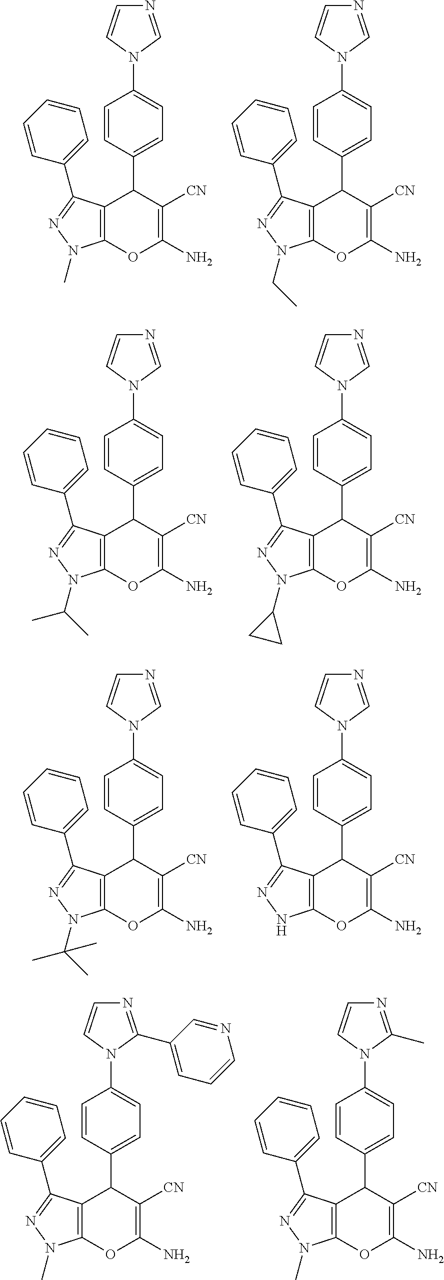

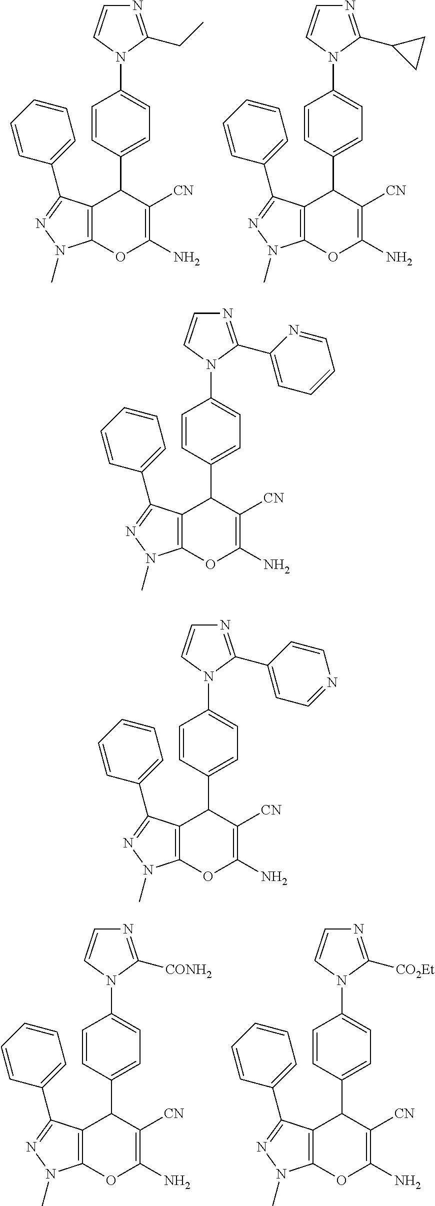

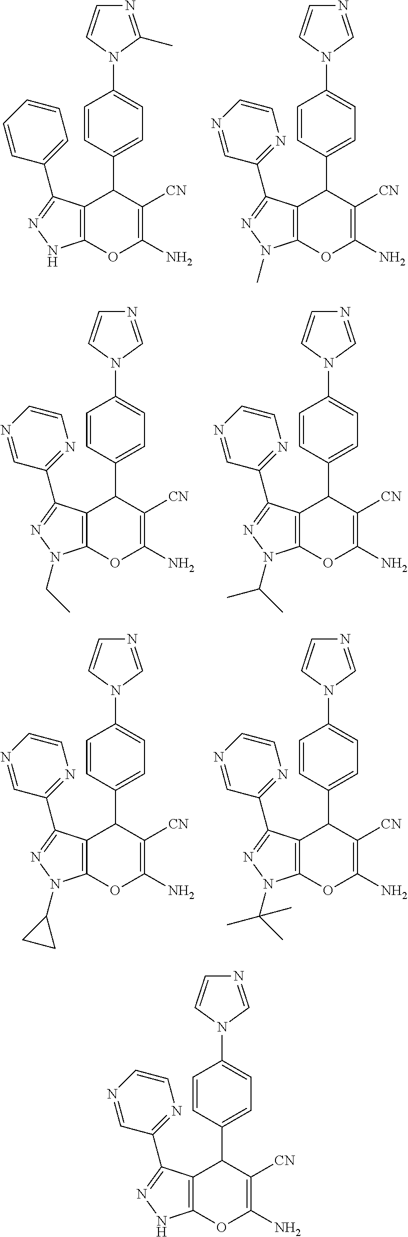

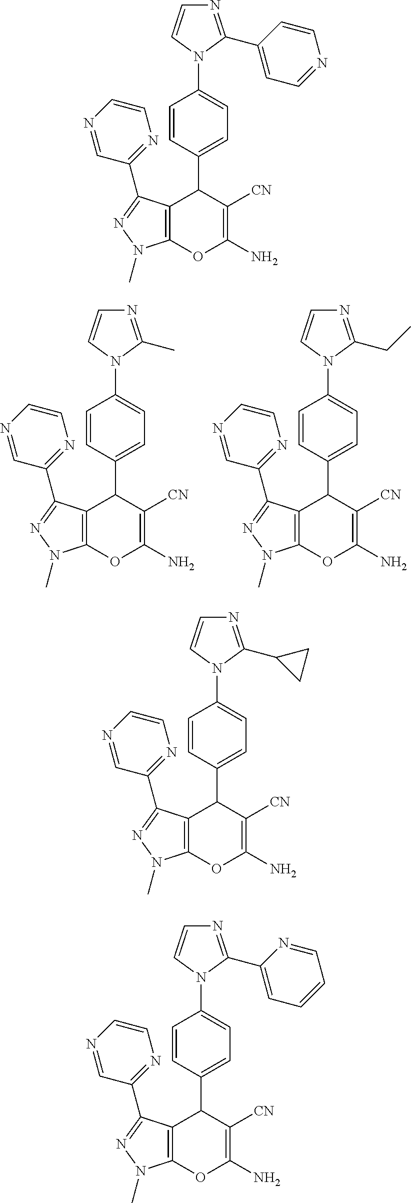

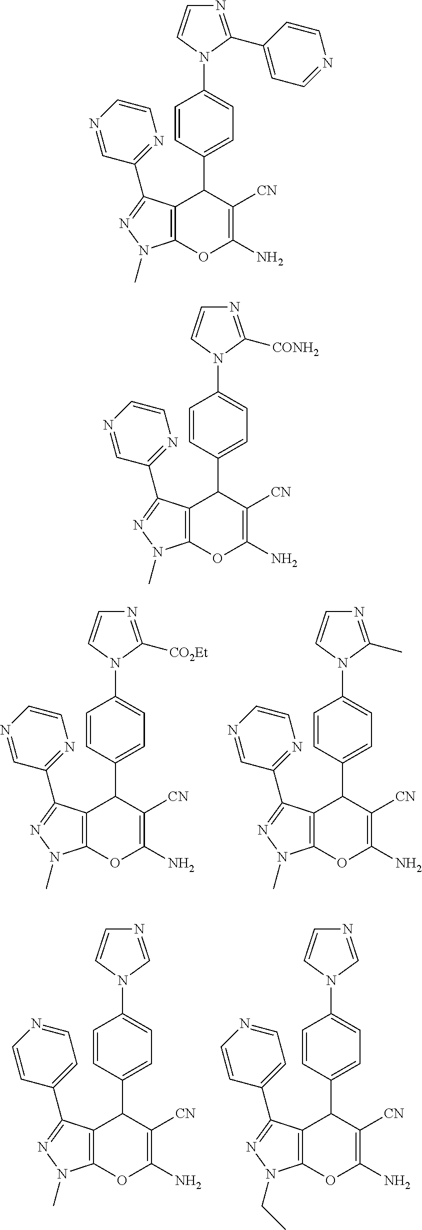

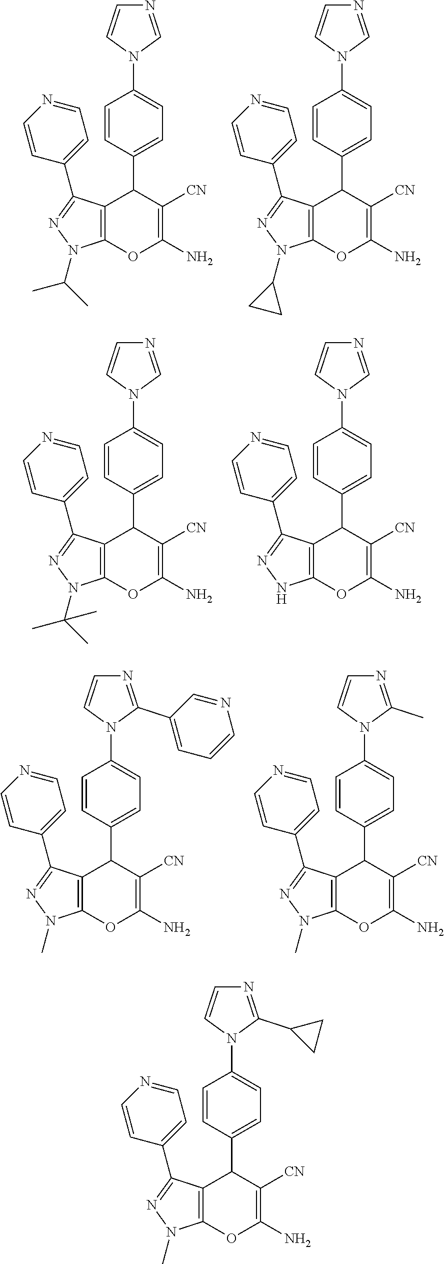

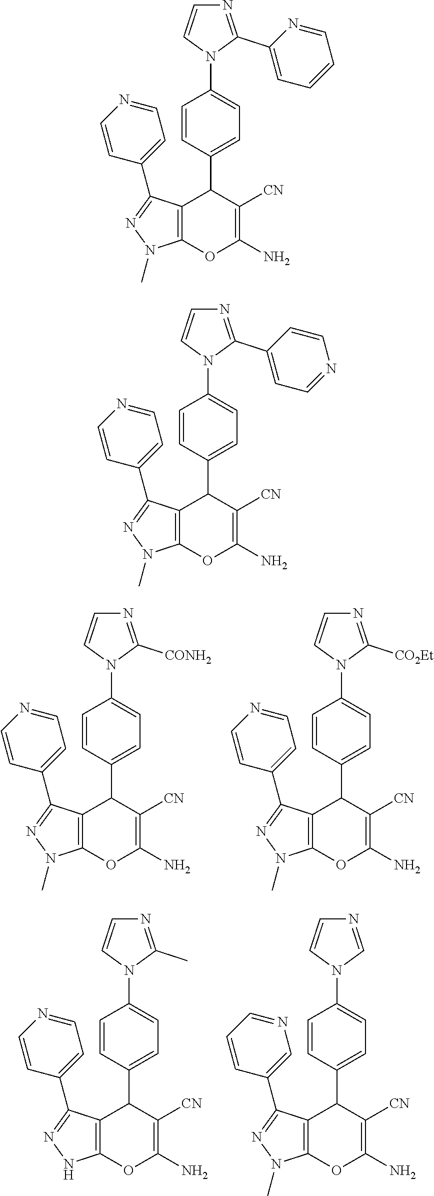

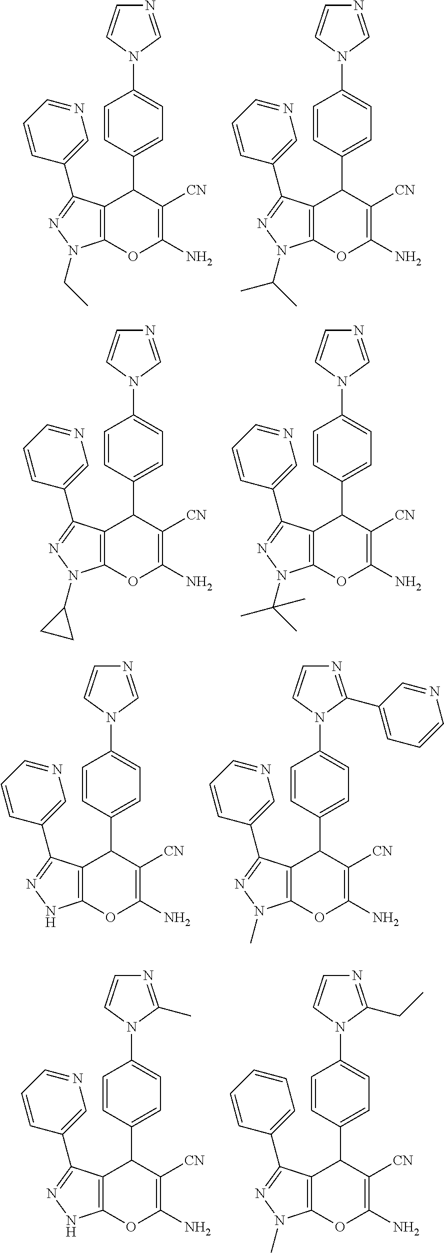

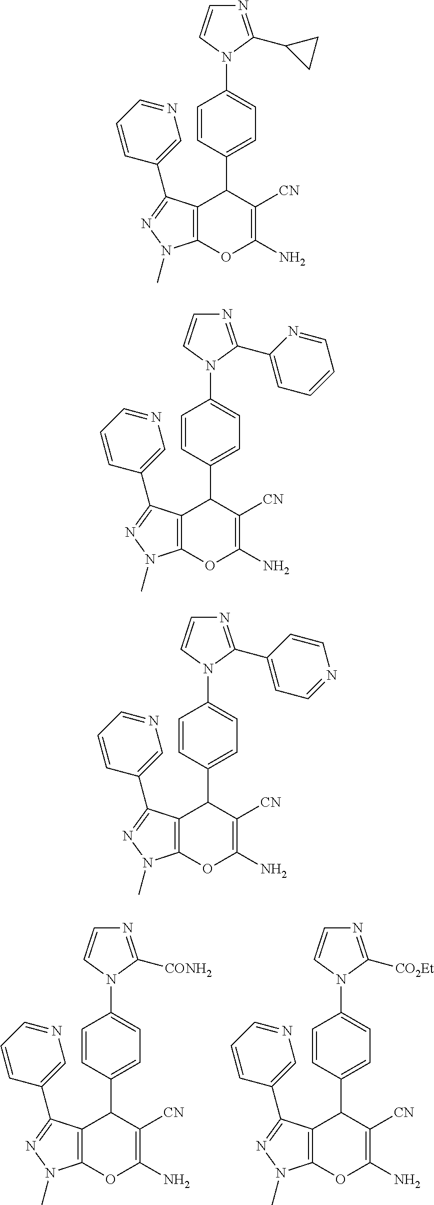

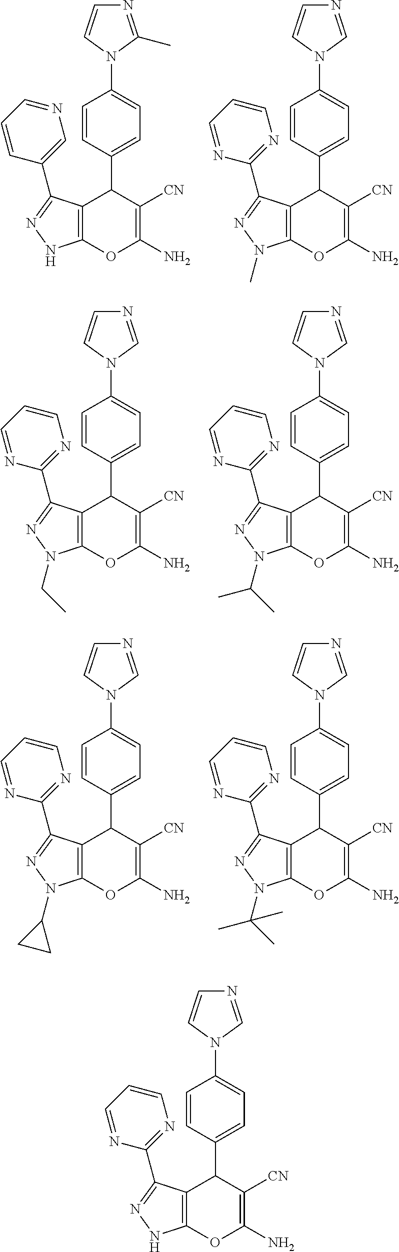

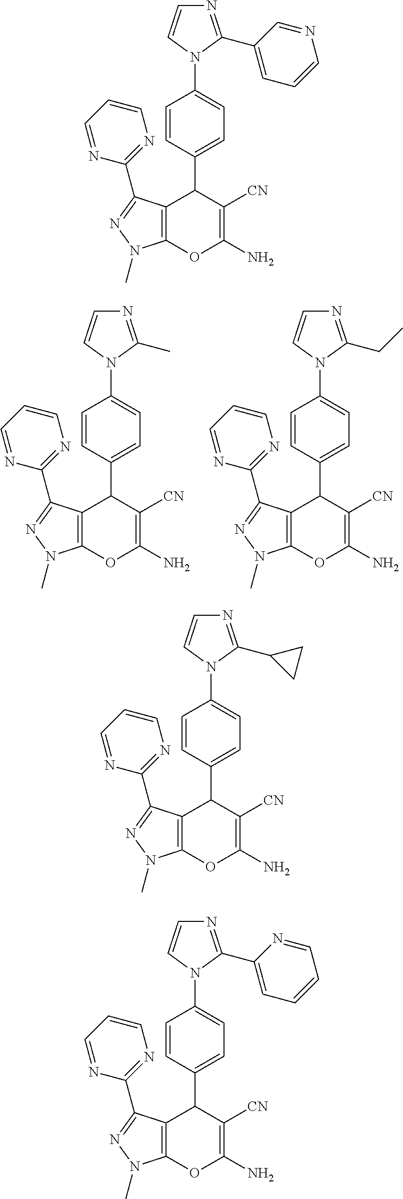

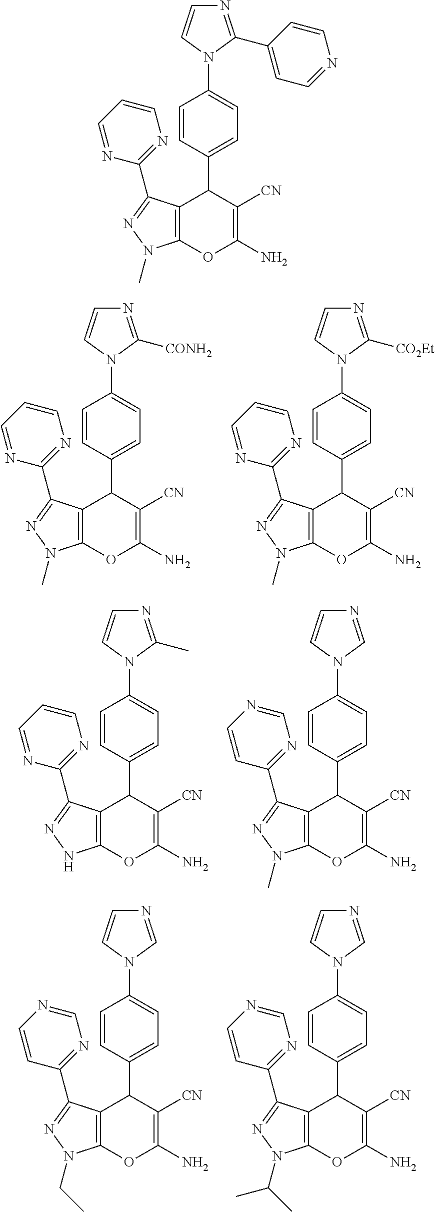

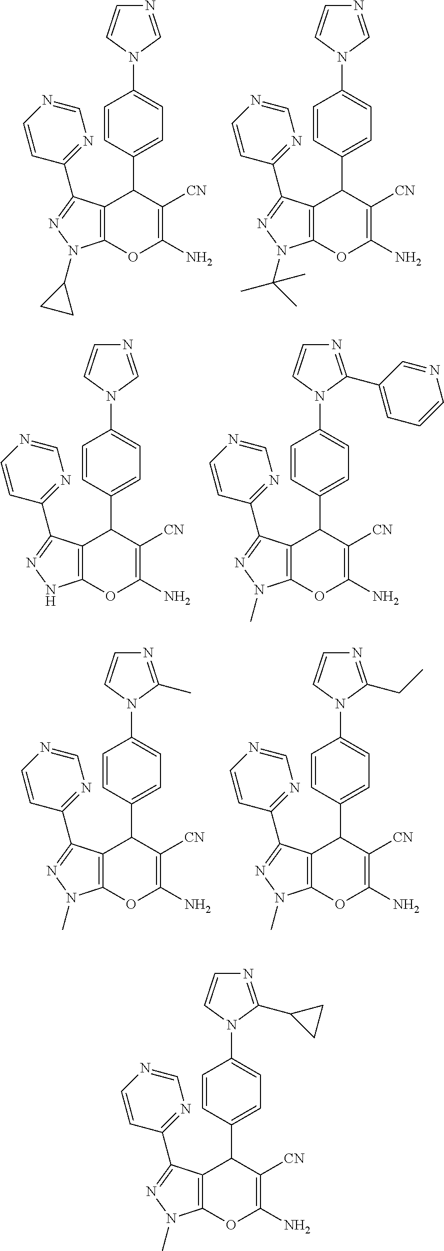

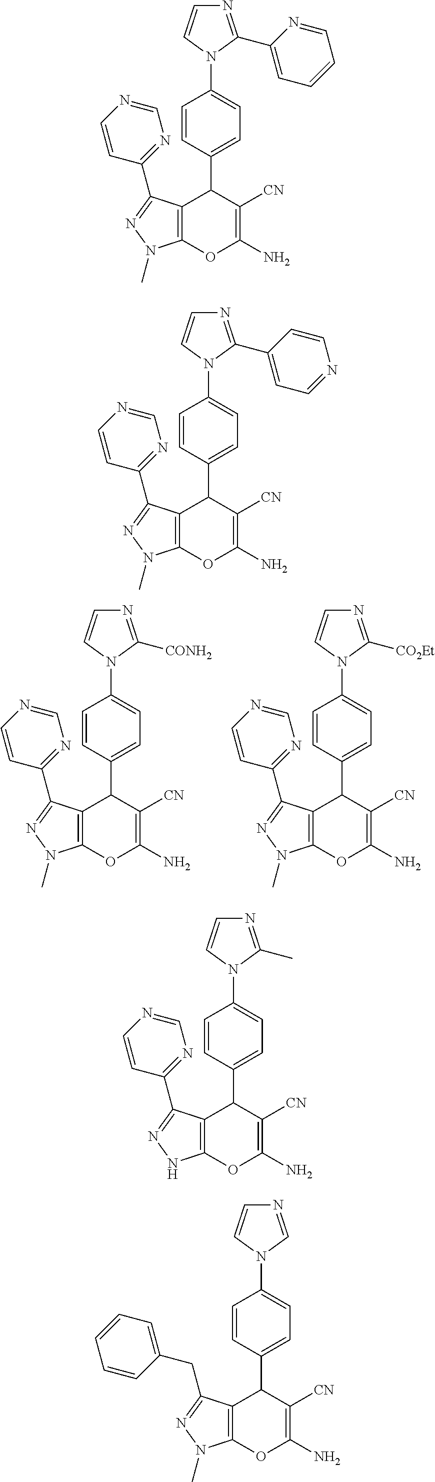

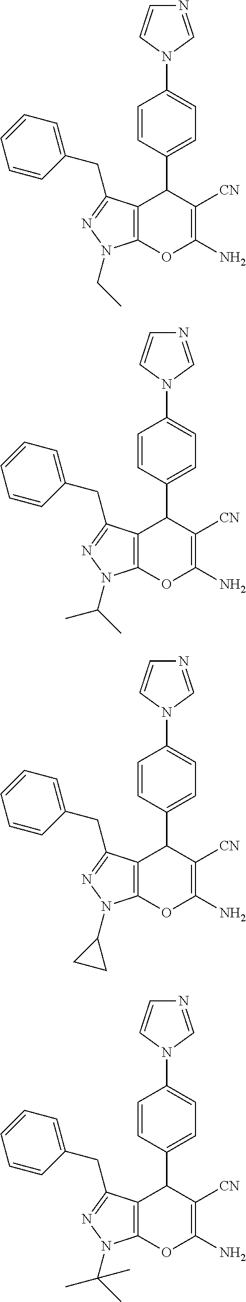

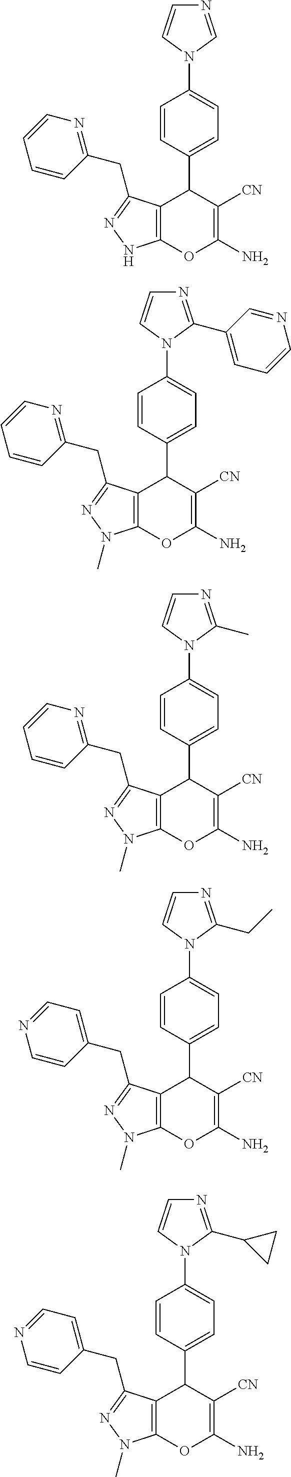

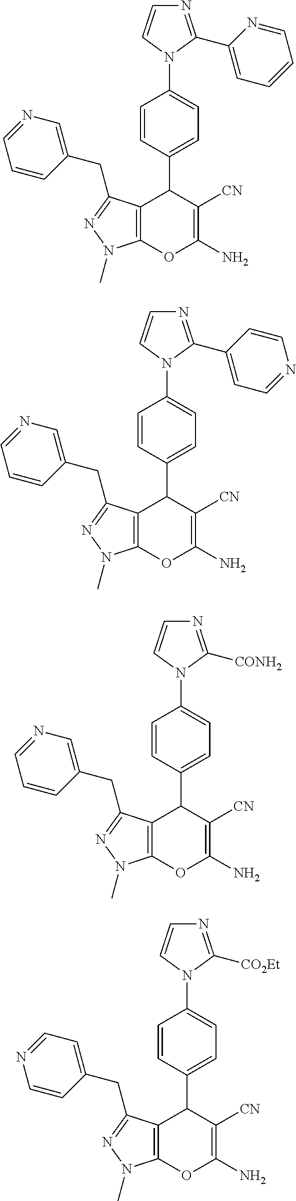 [SMILES: Cn1nc(Cc2cccnc2)c2c1OC(N)=C(C#N)C2c1ccc(-n2ccnc2-c2ccccn2)cc1 ; Cn1nc(Cc2cccnc2)c2c1OC(N)=C(C#N)C2c1ccc(-n2ccnc2-c2ccncc2)cc1 ; Cn1nc(Cc2cccnc2)c2c1OC(N)=C(C#N)C2c1ccc(-n2ccnc2C(N)=O)cc1 ; CCOC(=O)c1nccn1-c1ccc(C2C(C#N)=C(N)Oc3c2c(Cc2ccncc2)nn3C)cc1]

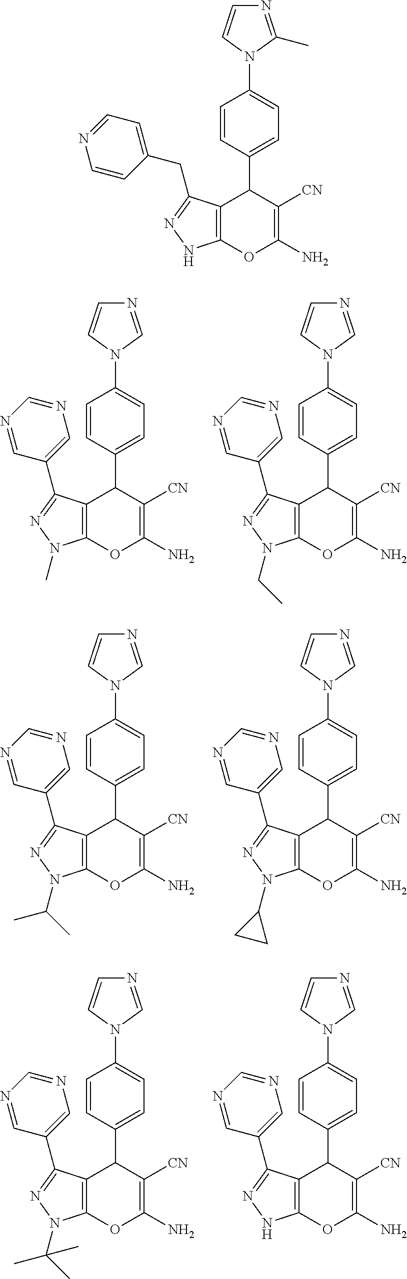

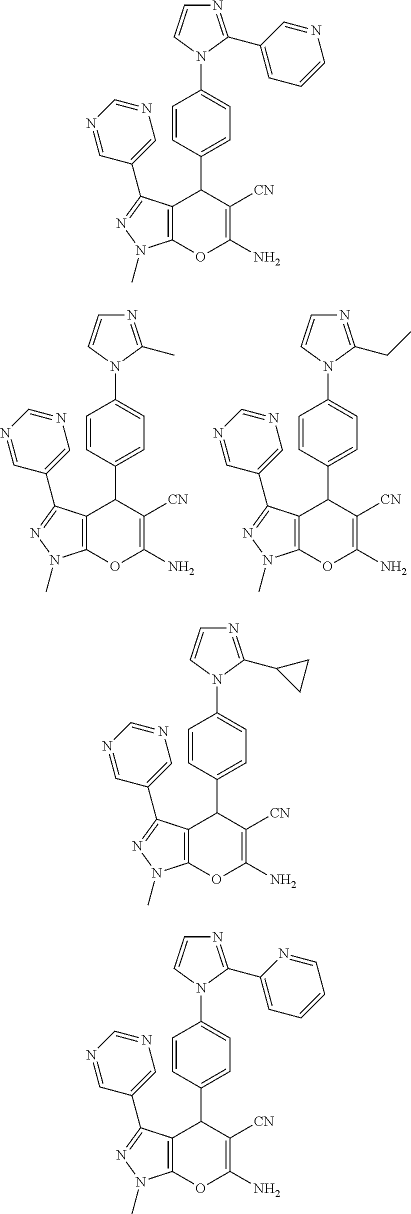

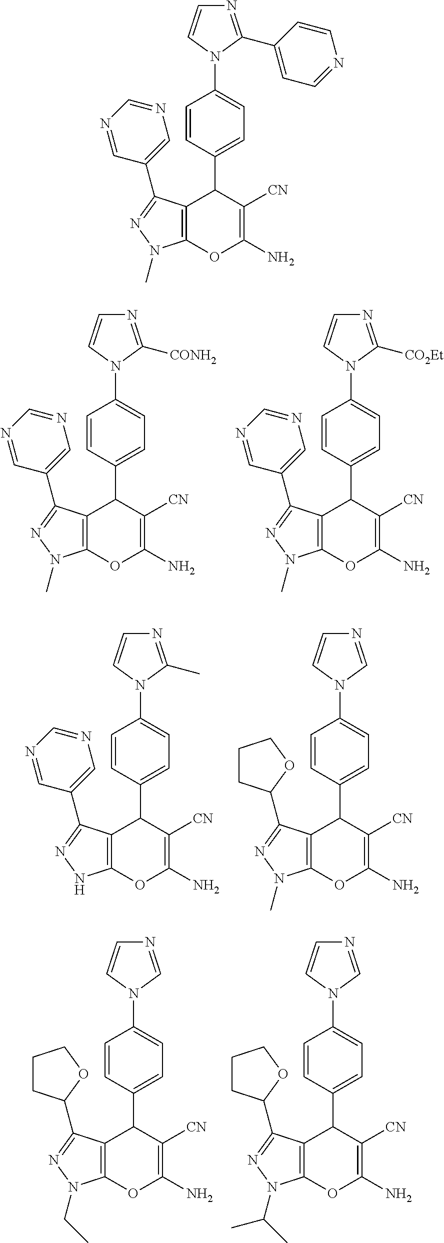

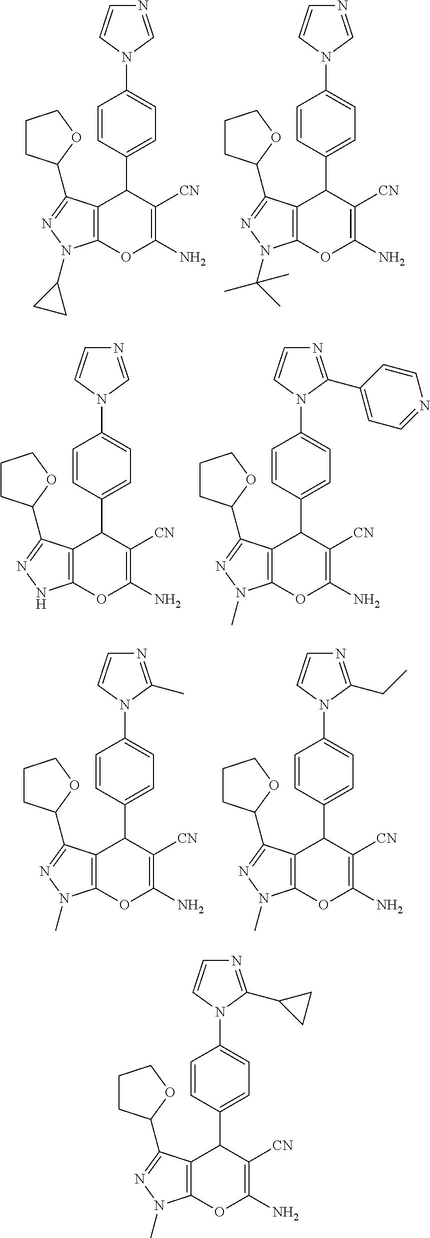

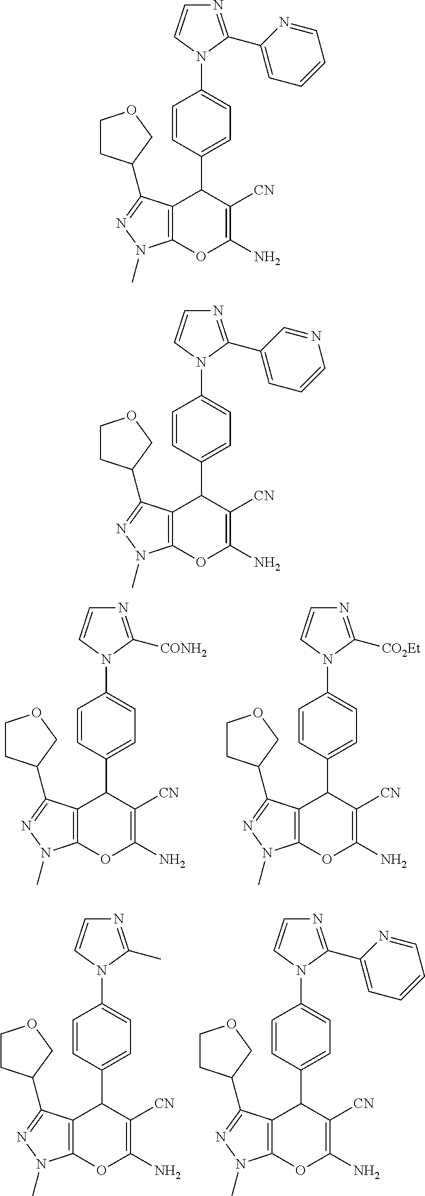

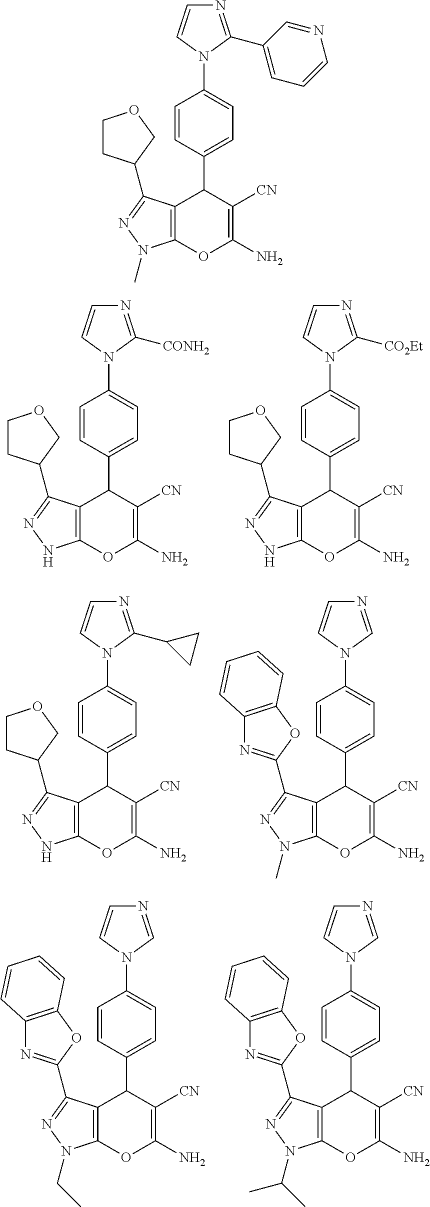

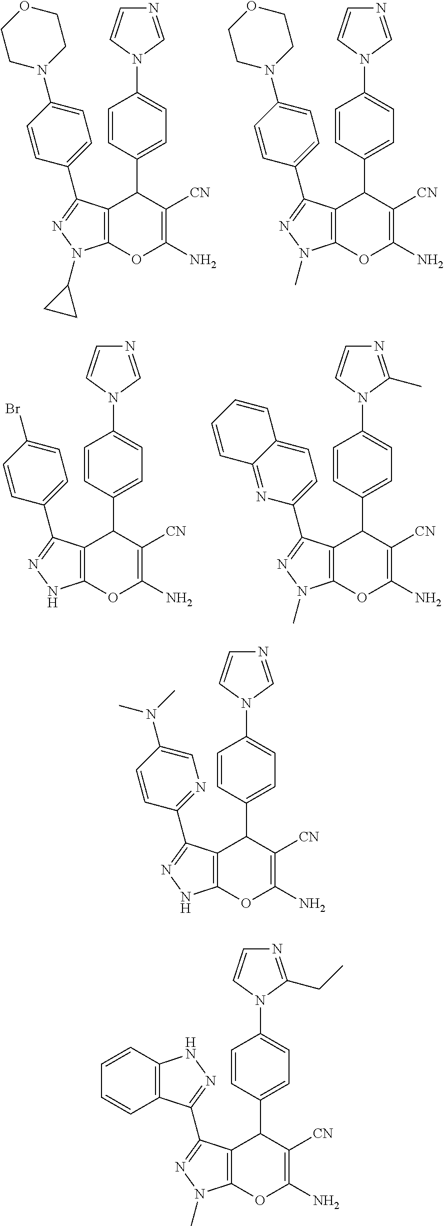

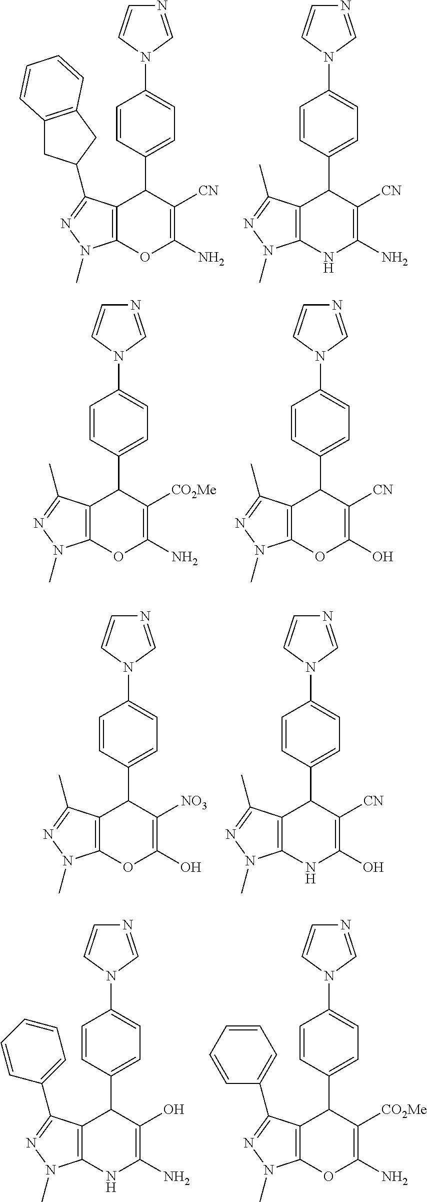

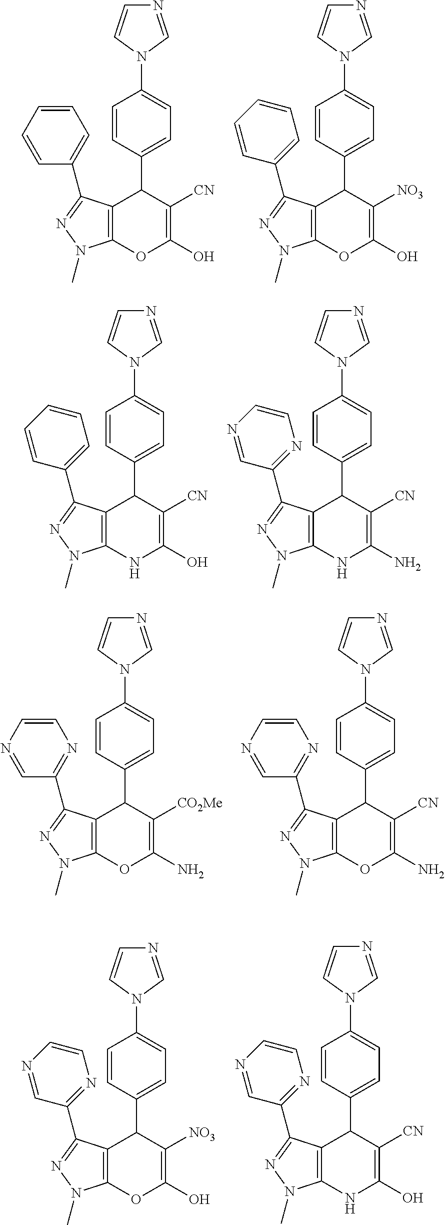

For compounds having asymmetric centers, it should be understood that (unless otherwise specified) all of the optical isomers and mixtures thereof are encompassed. In addition, compounds with carbon-carbon double bonds may occur in Z- and E-forms, with all isomeric forms of the compounds being included in the present invention unless otherwise specified. Where a compound exists in various tautomeric forms, a recited compound is not limited to any one specific tautomer, but rather is intended to encompass all tautomeric forms. Certain compounds are described herein using a general formula that include, variables (e.g. X, Ar.). Unless otherwise specified, each variable within such a formula is defined independently of any other variable, and any variable that occurs more than one time in a formula is defined independently at each occurrence.

It will be appreciated by those skilled in the art that compounds of the invention having a chiral center may exist in, and may be isolated in, optically active and racemic forms. It is to be understood that the compounds of the present invention encompasses any racemic, optically-active, regioisomeric or stereoisomeric form, or mixtures thereof, which possess the therapeutically useful properties described herein. Where the compounds of the invention have at least one chiral center, they may exist as enantiomers. Where the compounds possess two or more chiral centers, they may additionally exist as diastereomers. Where the processes for the preparation of the compounds according to the invention give rise to mixtures of stereoisomers, these isomers may be separated by conventional techniques such as preparative chromatography. The compounds may be prepared in racemic form or as individual enantiomers or diasteromers by either stereospecific synthesis or by resolution. The compounds may, for example, be resolved into their component enantiomers or diasteromers by standard techniques, such as the formation of stereoisomeric pairs by salt formation with an optically active acid, such as (−)-di-p-toluoyl-D-tartaric acid and/or (+)-di-p-toluoyl-L-tartaric acid followed by fractional crystallization and regeneration of the free base. The compounds may also be resolved by formation of stereoisomeric esters or amides, followed by chromatographic separation and removal of the chiral auxiliary. Alternatively, the compounds may be resolved using a chiral HPLC column. It is to be understood that all stereoisomers, racemic mixtures, diastereomers and enantiomers thereof are encompassed within the scope of the present invention.

It is well known in the art how to prepare optically active forms (for example, by resolution of the racemic form by recrystallization techniques, by synthesis from optically-active starting materials, by chiral synthesis, or by chromatographic separation using a chiral stationary phase). It is also to be understood that the scope of this invention encompasses not only the various isomers, which may exist but also the various mixtures of isomers, which may be formed. The resolution of the compounds of the present invention, their starting materials and/or the intermediates may be carried out by known procedures, e.g., as described in the four volume compendium Optical Resolution Procedures for Chemical Compounds: Optical Resolution Information Center, Manhattan College, Riverdale, N.Y., and in Enantiomers, Racemates and Resolutions, Jean Jacques, Andre Collet and Samuel H. Wilen; John Wiley & Sons, Inc., New York, 1981, which is incorporated in its entirety by this reference. Basically, the resolution of the compounds is based on the differences in the physical properties of diastereomers by attachment, either chemically or enzymatically, of an enantiomerically pure moiety resulting in forms that are separable by fractional crystallization, distillation or chromatography.

Moreover, it should also be appreciated that contemplated compounds may be prepared in a prodrug form to so achieve a desired pharmaceutical, pharmacokinetic, and/or pharmacodynamic parameter. The term “prodrug” as used herein refers to a modification of contemplated compounds, wherein the modified compound exhibits less pharmacological activity as compared to the unmodified compound, and wherein the modified compound is converted within a target cell (e.g., cancer cell) or target organ/anatomic structure (e.g., pancreas) back into the unmodified form. For example, conversion of contemplated compounds into prodrugs may be useful where the active drug is too toxic for safe systemic administration, or where the contemplated compound is poorly absorbed by the digestive tract or other compartment or cell, or where the body breaks down the contemplated compound before reaching its target. Thus, it should be recognized that the compounds according to the inventive subject matter can be modified in numerous manners, and especially preferred modifications include those that improve one or more pharmacokinetic and/or pharmacodynamic parameter. For example, one or more substituents may be added or replaced to achieve a higher AUC in serum.

On the other hand, and especially where increased solubility is desired, hydrophilic groups may be added. Still further, where contemplated compounds contain one or more bonds that can be hydrolyzed (or otherwise cleaved), reaction products are also expressly contemplated. Exemplary suitable protocols for conversion of contemplated compounds into the corresponding prodrug form can be found in “Prodrugs (Drugs and the Pharmaceutical Sciences: a Series of Textbooks and Monographs)” by Kenneth B. Sloan (ISBN: 0824786297), and “Hydrolysis in Drug and Prodrug Metabolism: Chemistry, Biochemistry, and Enzymology” by Bernard Testa, Joachim M. Mayer (ISBN: 390639025X), both of which are incorporated by reference herein. Moreover, especially where contemplated compounds have a higher activity when the compound is metabolized (e.g., hydrolyzed, hydroxylated, glucuronidated, etc.), it should be appreciated that metabolites of contemplated compounds are also expressly contemplated herein.

General Synthetic Procedures

In another embodiment, a method of preparing the invented compounds is provided. The compounds of the present invention can be generally prepared by coupling key intermediates II and III via established condensation procedures. Compound (I) and or (Ia) may contain various stereoisomers, geometric isomers, tautomeric isomers, and the like. All of possible isomers and their mixtures are included in the present invention, and the mixing ratio is not particularly limited.

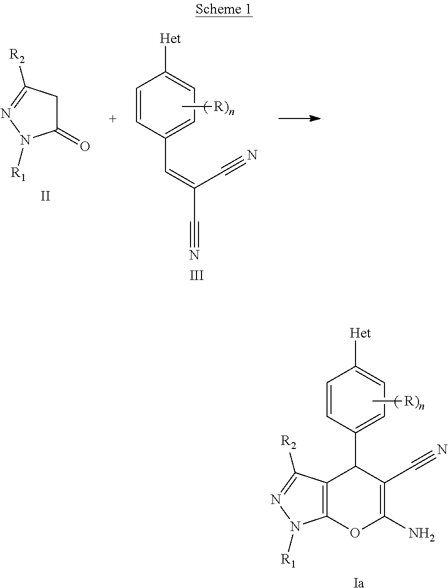

Methodologies for synthesis of functionalized pyrano[2,3-c]pyrazoles has been reviewed recently (Synthetic Developments in Functionalized Pyrano[2,3-c]pyrazoles. A Review. Bekington Myrboh, Hormi Mecadon, Md. Rumum Rohman, Mantu Rajbangshi, Icydora Kharkongor, Badaker M. Laloo, Iadeishisha Kharbangar, and Baskhemlang Kshiar Organic Preparations and Procedures International, 45:253-303, 2013).

Syntheses of 1,4-dihydropyrano[2,3-c]pyrazole compounds of general formula (Ia) are preferably carried out via strategies described in Scheme 1. Condensation of pyrazolone II with substituted 2-benzylidenemalononitrile intermediate III in the presence of N-methyl morpholine in ethanol with or without heating could generally afford the desired product.

Intermediate II could be prepared via strategies described in Scheme 2. Thus, condensation of substituted hydrazine IV with an equivalent substituted malonate V in ethanol with or without reflux could afford the desired product.

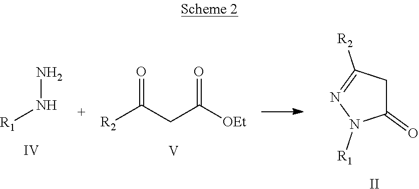

General methods to make intermediates of formula III are described in Scheme 3. Knoevengal condensation of malonitrile with heterocycle substitute aryl aldehyde VI in the presence of N-methyl morpholine in ethanol quantitatively affords the desire product.

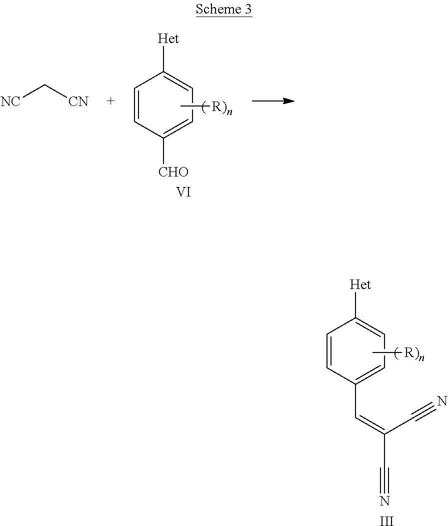

Alternatively, compounds Ia can be made under one-pot conditions at room temperature by mixing malonitrile with intermediate VI followed by treatment with equivalent intermediate II in ethanol (Three-Component Combinatorial Synthesis of Novel Dihydropyrano[2,3-c]pyrazoles. Lehmann F, Holm M, and Laufer S J. Comb. Chem. 2008, 10, 364-367).

Aldehydes such as in the formula of VI could be prepared using several methods. For the ones with C-linked heterocycles, the synthesis could be performed under Suzuki-Miyaura cross-coupling reaction conditions (Chapoulaud, V. G. et al., Tetrahedron, 2000, 56, 5499-5507; Mongin, F., Rebstock, A., et al., J. Org. Chem., 2004, 69, 6766-6771). The coupling of various heteroaryl boronic acids or the relevant pinacol boronates with bromides VII affords the compounds II in the presence of an appropriate palladium catalyst, such as palladium(II) acetate triphenylphosphine, dichlorobis(triphenylphosphine)palladium(0), or tetrakis(triphenylphosphine)palladium(0). The reaction also works with pseudohalides such as triflates (OTO, instead of halides, and also with boron-esters instead of boronic acids. A variety of base agent may be used, but not limited to, KOAC, K₂CO₃, K₃PO₄, KOH, NaOH, KF, NaOAc, Na₂CO₃, Cs₂CO₃, NaHCO₃ and the like. The suitable solvent may be used, but not limited to, dioxane, acetonitrile, THF, DMF, DMSO, THF, toluene and the like, may be used alone or as a mixture thereof, conveniently at a temperature within the range room temperature to reflux.

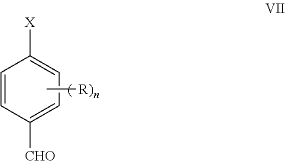

For the ones with N-linked heterocycles such as lactam, the synthesis could be performed under Buchwald-Hartwig amination conditions. The coupling of various heteroaryl with nucleophilic nitrogens with bromides VII affords the compounds II in the presence of an appropriate palladium catalyst, such as palladium(II) acetate, dichlorobis(triphenylphosphine)palladium(0), or tetrakis(triphenylphosphine)palladium(0). A variety of base agent may be used, but not limited to, KOAC, K₂CO₃, K₃PO₄, KOH, NaOH, KF, NaOAc, Na₂CO₃, Cs2CO3, NaHCO₃ and the like. The suitable solvent may be used, but not limited to, DME, dioxane, acetonitrile, THF, DMF, DMSO, THF, toluene and the like, may be used alone or as a mixture thereof, conveniently at a temperature within the range room temperature to reflux.

Alternatively, syntheses of heteroaryl aldehyde VI with N-linked five membered heterocycles such as imidazole and triazole could be carried out via the strategies as described in Scheme 4.

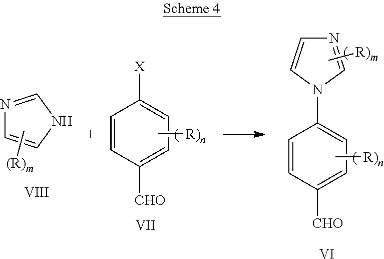

For example, coupling of substituted imidazoles in the formula of VIII with aldehydes VII in the presence of Hunig's base in acetonitrile under reflux could afford the desired products in moderate to good yields (Huang Zhangjiang, Liu Bingni, Liu Dengke, Liu Mo, Liu Ying, Yang Miao, Zhang Zhiqiang and Zou Meixiang, A class of imidazole derivatives, their preparation and use, CN101781294).

Alternatively, synthesis of Syntheses of 1,4-dihydropyrano[2,3-c]pyrazole compounds of general formula (Ia) could be carried out via a reaction between 3-methyl-1-phenyl-2-pyrazolin-5-one II, aromatic aldehydes VI and malononitrile using tungstate sulfuric acid as a catalyst (An environmentally friendly synthesis of 1,4-dihydropyrano[2,3-c]pyrazole derivatives catalyzed by tungstate sulfuric acid Farahi M, Karami B, Sedighimehr I, Tanuraghaj H M, Chinese Chemical Letters 25 (2014) 1580-1582) or using MgO as the catalyst (Three-Component Reaction to Form 1,4-Dihydropyrano[2,3-c]pyrazol-5-yl Cyanides, Synthetic Communications: An International Journal for Rapid Communication of Synthetic Organic Chemistry, Sheibani H and Babaie M. 40:2, 257-265).

Syntheses of 4,7-dihydro-1H-pyrazolo[3,4-b]pyridine compounds of general formula (Ib) are preferably carried out via strategies described in Scheme 5. Thus, the three component condensation of intermediates in the formula of II and VI and malonitrile can be performed in the presence of ammonium acetate in ethanol under reflux conditions (Facile synthesis of fused nitrogen containing heterocycles as anticancer agents. By: Mohamed, Nadia R.; Khaireldin, Nahed Y.; Fahmy, Amin F.; El-Sayed, Ahmed A. Pharma Chemica, 2(1), 400-417; 2010).

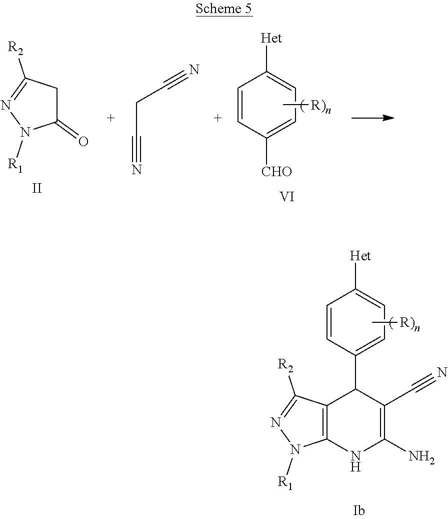

Syntheses of compounds of general formula (Ic) are preferably carried out via strategies described in Scheme 6. A four-component condensation of intermediates of general formula IV, V, VI and cyanocarboxylates IX could be carried out via catalysis of freshly prepared SnO₂ nanoflower. The preparation of SnO₂ QD was recently reported (Uncapped SnO quantum dot catalyzed cascade assembling of four components: a rapid and green approach to the pyrano[2,3-c]pyrazole and spiro-2-oxindole derivatives. Paul S, Pradhan K, Ghosh S, Das A R. Tetrahedron 70 (2014) 6088-6099).

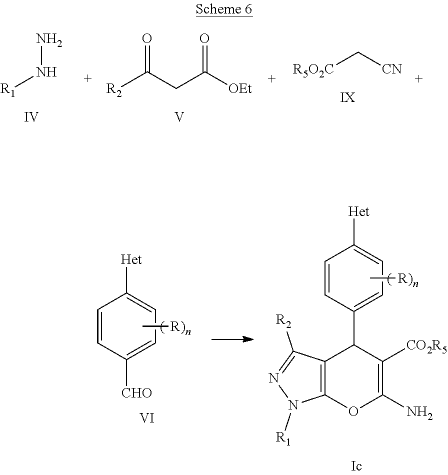

Syntheses of compounds of general formula (Id) are preferably carried out via strategies described in Scheme 7. A condensation of pyrazolone intermediate II with ethyl arylidenecyanoacetates in a formula of X could be efficiently carried out under refluxing conditions in the presence of piperidine catalysts (G. E. H. Elgemeie, B. Y. Riad, G. A. Nawwar and S. Elgamal, Arch. Pharm. Chem., (Weinheim), 320, 223 (1987); Chem. Abstr., 107, 198239 (1987).

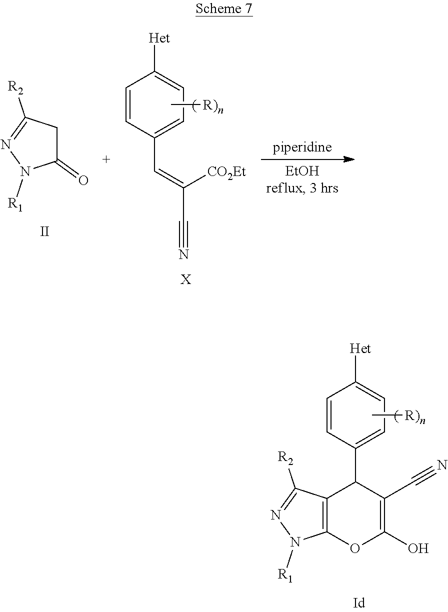

Syntheses of compounds of general formula (Ie) are preferably carried out via strategies as described in Scheme 8. Thus, condensation of arylidenenitroacetonitriles in a formula of XI with pyrazolone intermediate II (A. S. Polyanskaya, R. I. Bodina, V. Y. Shchadrin and N. I. Aboskalova, Zh. Org. Khim, 1984, 20, 2481; Chem. Abstr., 1985, 102, 149167).

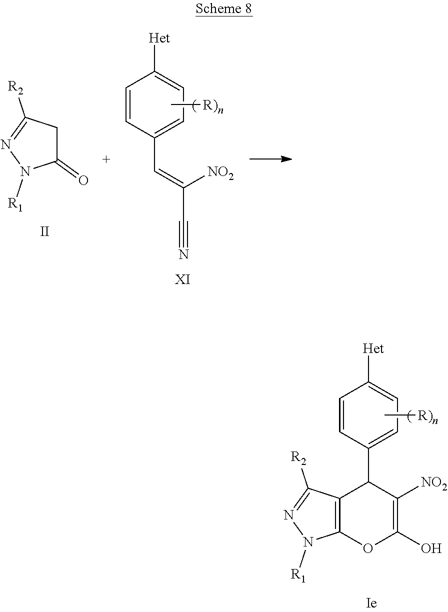

Syntheses of compounds of general formula (If) are preferably carried out via strategies as described in Scheme 9. Thus, compounds in a formula of Ia could be converted directly into If upon treatment with a mixture of acetic acid and sulphuric acid under reflux conditions (see reference, Catalyst free, one-pot, facile synthesis of novel pyrazolo-1,4-dihydropyridine derivative form pyranopyrazoles. Sohal H. S., Goyal A., Khare R., Singh K. and Sharma R. European Journal of Chemistry, 2014, 5, 227-232)

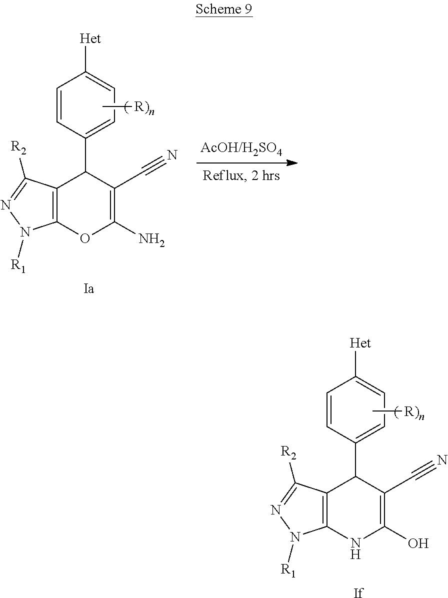

One embodiment of the invention is a method of treating a cancer by administering to a mammal in need of such treatment, a therapeutically-effective amount of a compound that inhibits Ral GTPase enzymatic activity. In one aspect of this embodiment, the compound inhibits at least one paralog of Ral GTPAse (either RalA or RalB), thereby inhibiting the growth or metastasis of a cancer. In a preferred aspect of this embodiment, the compound inhibits both RalA and RalB paralogs.

In a specific embodiment of these methods of treating or preventing a cancer in a mammal, the compound is administered to the mammal within a pharmaceutical composition of the invention.

Another embodiment of the invention is a method of preventing or treating metastatic cancers, particularly metastatic pancreas, prostate, lung, bladder, and/or colon cancers, by administering a therapeutically effective amount of at least one compound of the invention to a mammal in need of such treatment or suspected of having a cancer or a metastasis of a cancer.

Another embodiment of the invention is a method of treating cancer by administering a therapeutically effective combination of at least one of the compounds of the invention and one or more other known anti-cancer or anti-inflammatory treatments. For example, other anti-cancer treatments may include prenyltransferase inhibitors, including geranylgeranyltransferase type I (GGTase-I) inhibitors, surgery, chemotherapy, radiation, immunotherapy, or combinations thereof.

Also provided herein are methods for the prevention, treatment or prophylaxis of cancer in a mammal comprising administering to a mammal in need thereof, therapeutically-effective amounts of any of the pharmaceutical compositions of the invention.

Also provided herein are methods for preventing the metastasis of a cancer in a mammal comprising administering to the mammal, therapeutically-effective amounts of at least one compound of the invention, including, for example, pharmaceutical compositions containing at least one compound of the invention.

Also provided herein are pharmaceutical packages comprising therapeutically-effective amounts of at least one compound of the invention within a pharmaceutical composition. The pharmaceutical compositions may be administered separately, simultaneously or sequentially with other compounds or therapies used in the prevention, treatment or amelioration of cancer in a mammal. These packages may also include prescribing information and/or a container. If present, the prescribing information may describe the administration, and/or use of these pharmaceutical compositions alone or in combination with other therapies used in the prevention, treatment or amelioration of cancer in a mammal.

Another embodiment of this invention is a method of testing the susceptibility of a mammal having lung cancer to treatment with a putative inhibitor of Ral GTPase activity by testing the mammal for a response to administration of the putative inhibitor indicative of growth inhibition or reduction in cancer cell number or tumor volume in the mammal.

### EXAMPLES

The following examples are provided to further illustrate the present invention but, of course, should not be construed as in any way limiting its scope.

All experiments were performed under anhydrous conditions (i.e. dry solvents) in an atmosphere of argon, except where stated, using oven-dried apparatus and employing standard techniques in handling air-sensitive materials. Aqueous solutions of sodium bicarbonate (NaHCO3) and sodium chloride (brine) were saturated.

Analytical thin layer chromatography (TLC) was carried out on Merck Kiesel gel 60 F254 plates with visualization by ultraviolet and/or anisaldehyde, potassium permanganate or phosphomolybdic acid dips.

NMR spectra: 1H Nuclear magnetic resonance spectra were recorded at 400 MHz. Data are presented as follows: chemical shift, multiplicity (s=singlet, d=doublet, t=triplet, q=quartet, qn=quintet, dd=doublet of doublets, m=multiplet, bs=broad singlet), coupling constant (J/Hz) and integration. Coupling constants were taken and calculated directly from the spectra and are uncorrected.

Low resolution mass spectra: Electrospray (ES+) ionization was used. The protonated parent ion (M+H) or parent sodium ion (M+Na) or fragment of highest mass is quoted. Analytical gradient consisted of 10% ACN in water ramping up to 100% ACN over 5 minutes unless otherwise stated.

High performance liquid chromatography (HPLC) was use to analyze the purity of derivatives. HPLC was performed on a Phenomenex Synergi Polar-RP, 4u, 80A, 150×4.6 mm column using a Shimadzu system equipped with SPD-M10A Phosphodiode Array Detector. Mobile phase A was water and mobile phase B was acetonitrile with a gradient from 20% to 80% B over 60 minutes and re-equilibrate at A/B (80:20) for 10 minutes. UV detection was at 220 and 54 nm.

Preparation of Exemplary Intermediates

Intermediate 1

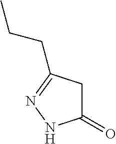

To a solution of ethyl 3-oxohexanoate (6.74 g, 42.64 mmol) in anhydrous ethanol (100 mL) was added dropwise a solution of hydrazine (42.6 mL, 42.64 mmol) in THF (1 N) at 0° C. The reaction was allowed to be warmed up to room temperature and stirred for 20 hrs. The mixture was further heated at 60° C. for 4 hrs. Reaction mixture was concentrated on rotavapor to dryness to provide intermediate 1 as a pale yellow solid (5.37 g, 100%). ¹H NMR (400 MHz, DMSO-d₆) δ (ppm): 5.21 (s, 1H), 4.06 (dd, J=7.2, 1.2 Hz, 1H), 3.52 (d, J=1.2 Hz, 1H), 2.47-2.38 (m, 2H), 1.58-1.40 (m, 2H), 1.18-1.14 (m, 2H), 0.88-0.82 (m, 3H). MS (ESI): Calcd for C6H11N2O: 127.1, found: 127.2 (M+H)⁺.

Intermediate 2

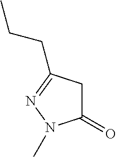

To a solution of ethyl 3-oxohexanoate (10.72 g, 67.87 mmol) in anhydrous ethanol (100 mL) was added slowly methyl hydrazine (3.57 mL, 67.87 mmol) at 0° C. The reaction was allowed to be warmed up to room temperature for 3.5 hrs. The mixture was further heated at 60° C. for 10 hrs. and kept reflux for 2.5 hrs. Reaction mixture was concentrated on rotavapor to dryness to provide intermediate 2 as a viscous pink solid (9.50 g, 100%). ¹H-NMR (400 MHz, CDCl₃) δ (ppm): 3.25 (s, 3H), 3.13 (d, J=0.8 Hz, 2H), 2.34 (t, J=7.6 Hz, 2H), 1.59 (dd, J=14.8, 7.2 Hz, 2H), 0.95 (t, J=7.2 Hz, 3H). MS (ESI): Calcd for C7H13N2O: 141.1, found: 141.1 (M+H)⁺.

Intermediate 3

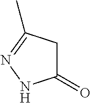

To a solution of ethyl 3-oxohexanoate (3.07 g, 23.6 mmol) in anhydrous ethanol (100 mL) was added dropwise a solution of hydrazine (23.6 mL, 23.6 mmol) in THF (1 N) at 0° C. The reaction was allowed to be warmed up to room temperature and stirred for 20 hrs. The mixture was further heated at 60° C. for 6 hrs. Reaction mixture was concentrated on rotavapor to dryness to provide intermediate 3 as an off white solid (2.31 g, 100%). ¹H NMR (400 MHz, CDCl3) δ (ppm): 10.0 (bs, 1H), 3.32 (s, 2H), 2.20 (s, 3H). MS (ESI): Calcd for C4H7N2O: 99.1, found: 99.1 (M+H)⁺.

Intermediate 4

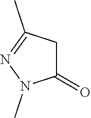

To a solution of ethyl 3-oxohexanoate (11.06 g, 84.99 mmol) in anhydrous ethanol (100 mL) was added slowly methyl hydrazine (4.47 mL, 84.99 mmol) at 0° C. The reaction was allowed to be warmed up to room temperature for 3.5 hrs. The mixture was further heated at 60° C. for 10 hrs. and kept reflux for 2.5 hrs. Reaction mixture was concentrated on rotavapor to dryness to provide intermediate 4 as a viscous pink solid (9.52 g, 100%). ¹H NMR (400 MHz, CDCl3) δ (ppm): 5.06 (s, 1H), 3.85 (bs, 1H), 3.32 (s, 3H), 1.94 (s, 3H). MS (ESI): Calcd for C5H9N2O: 113.1, found: 113.2 (M+H)⁺.

Intermediate 5

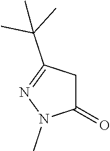

To a solution of ethyl 4,4-dimethyl-3-oxopentanoate (2.6 mL, 14.5 mmol) in anhydrous ethanol (15.0 mL) was added dropwise a solution of methylhydrazine (0.7 mL, 13.2 mmol) at 0° C. The reaction was allowed to be warmed up to room temperature and stirred for 3-5 hrs. The mixture was further heated at 60° C. overnight. Reaction mixture was concentrated on rotavapor to dryness to provide intermediate 5 as an off white solid (1.85 g). ¹H NMR (400 MHz, DMSO-d₆) δ (ppm): 10.62 (bs, 1H), 5.15 (s, 1H), 3.40 (s, 3H), 1.15 (s, 9H). MS (ESI): Calcd. for C8H14N2O: 154, found 155 (M+H)⁺.

Intermediate 6

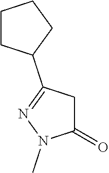

To a solution of ethyl 3-cyclopentyl-3-oxopropanoate (0.52 mL, 3.3 mmol) in anhydrous ethanol (5.0 mL) was added dropwise a solution of methylhydrazine (0.16 mL, 3 mmol) at 0° C. The reaction was allowed to be warmed up to room temperature and stirred for 3-5 hrs. The mixture was further heated at 60° C. over the weekend. Reaction mixture was concentrated on rotavapor to dryness to provide intermediate 6 as an off white solid (470 mg). ¹H NMR (400 MHz, DMSO-d₆) δ (ppm): 10.71 (bs, 1H), 5.11 (s, 1H), 3.38 (s, 3H), 2.78 (m, 1H), 1.87-1.52 (m, 8H). MS (ESI): Calcd. for C9H14N2O: 166, found 167 (M+H)⁺.

Intermediate 7

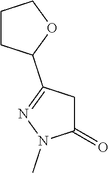

To a solution of ethyl 3-oxo-3-(tetrahydrofuran-2-yl)propanoate (0.49 mL, 3.3 mmol) in anhydrous ethanol (5.0 mL) was added dropwise a solution of methylhydrazine (0.16 mL, 3 mmol) at 0° C. The reaction was allowed to be warmed up to room temperature and stirred for 3-5 hrs. The mixture was further heated at 60° C. over the weekend. Reaction mixture was concentrated on rotavapor to dryness to provide intermediate 7 as a brown solid (530 mg). ¹H NMR (400 MHz, DMSO-d₆) δ (ppm): 5.22 (s, 1H), 4.55 (m, 1H), 3.80-3.42 (m, 2H), 3.13 (s, 3H), 2.05-1.86 (m, 4H). MS (ESI): Calcd. for C8H12N2O2: 168, found 169 (M+H)⁺.

Intermediate 8

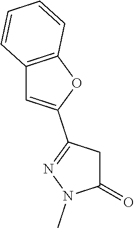 [SMILES: CN1N=C(c2cc3ccccc3o2)CC1=O]

To a solution of ethyl 3-(benzofuran-2-yl)-3-oxopropanoate (0.67 mL, 3.3 mmol) in anhydrous ethanol (5.0 mL) was added dropwise a solution of methylhydrazine (0.16 mL, 3 mmol) at 0° C. The reaction was allowed to be warmed up to room temperature and stirred for 3-5 hrs. The mixture was further heated at 60° C. over the weekend. Reaction mixture was concentrated on rotavapor to dryness to provide intermediate 8 as a white solid (550 mg). ¹H NMR (400 MHz, DMSO-d₆) δ (ppm): 11.31 (bs, 1H), 7.61-7.56 (m, 2H), 7.29-7.05 (m, 2H), 5.82 (s, 1H), 3.60 (s, 3H). MS (ESI): Calcd. for C12H10N2O2: 214, found 215 (M+H)⁺.

Intermediate 9

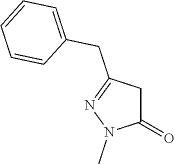

To a solution of ethyl 3-oxo-4-phenylbutanoate (0.57 mL, 3.3 mmol) in anhydrous ethanol (5.0 mL) was added dropwise a solution of methylhydrazine (0.16 mL, 3 mmol) at 0° C. The reaction was allowed to be warmed up to room temperature and stirred for 3-5 hrs. The mixture was further heated at 60° C. over the weekend. Reaction mixture was concentrated on rotavapor to dryness to provide intermediate 9 as a white solid (530 mg). ¹H NMR (400 MHz, DMSO-d₆) δ (ppm): 10.70 (bs, 1H), 7.26-7.19 (m, 5H), 5.08 (s, 1H), 3.67 (s, 2H), 3.42 (s, 3H). MS (ESI): Calcd. for C11H12N2O: 188, found 189 (M+H)⁺.

Intermediate 10

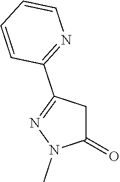

To a solution of ethyl 3-oxo-3-(pyridin-2-yl)propanoate (0.50 mL, 3.3 mmol) in anhydrous ethanol (5.0 mL) was added dropwise a solution of methylhydrazine (0.16 mL, 3 mmol) at 0° C. The reaction was allowed to be warmed up to room temperature and stirred for 3-5 hrs. The mixture was further heated at 60° C. over the weekend. Reaction mixture was concentrated on rotavapor to dryness to provide intermediate 10 as a pink solid (270 mg). ¹H NMR (400 MHz, DMSO-d₆) δ (ppm): 11.12 (bs, 1H), 8.51-8.50 (m, 1H), 7.84-7.30 (m, 2H), 7.25-7.22 (m, 1H), 5.92 (s, 1H), 3.59 (s, 3H). MS (ESI): Calcd. for C9H9N3O: 175, found 176 (M+H)⁺.

Intermediate 11

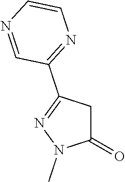

To a solution of ethyl 3-oxo-3-(pyrazin-2-yl)propanoate (0.58 mL, 3.3 mmol) in anhydrous ethanol (5.0 mL) was added dropwise a solution of methylhydrazine (0.16 mL, 3 mmol) at 0° C. The reaction was allowed to be warmed up to room temperature and stirred for 3-5 hrs. The mixture was further heated at 60° C. over the weekend. Reaction mixture was concentrated on rotavapor to dryness to provide intermediate 11 as a brown solid (350 mg). ¹H NMR (400 MHz, DMSO-d₆) δ (ppm): 11.32 (bs, 1H), 9.05 (s, 1H), 8.57-8.48 (m, 2H), 5.97 (s, 1H), 3.59 (s, 3H). MS (ESI): Calcd. for C8H8N4O: 176, found 177 (M+H)⁺.

Intermediate 12

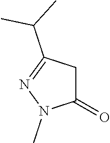

To a solution of isopropyl 3-oxohexanoate (12.11 g, 76.6 mmol) in anhydrous ethanol (150 mL) was added dropwise a solution of methylhydrazine (4.0 mL, 76.6 mmol) in THF (1 N) at 0° C. The reaction was allowed to be warmed up to room temperature and stirred for 28 hrs. The mixture was further heated under reflux for 8 hrs. Reaction mixture was concentrated on rotavapor to dryness to provide intermediate 12 as an orange solid (10.72 g, 100%). ¹H NMR (400 MHz, DMSO-d₆) δ (ppm): 10.70 (s, 1H), 5.10 (s, 1H), 3.36 (s, 3H), 2.65-2.61 (m, 1H), 1.07 (t, J=7.2 Hz, 6H). MS (ESI): Calcd for C7H13N2O: 141.1, found: 141.2 (M+H)⁺.

Intermediate 13

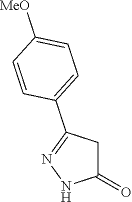 [SMILES: COc1ccc(C2=NNC(=O)C2)cc1]

To a solution of ethyl 3-(4-methoxyphenyl)-3-oxopropanoate (0.67 mL, 3.5 mmol) in anhydrous ethanol (5.0 mL) was added dropwise a solution of hydrazine, 1 M in THF (3.5 mL, 3.5 mmol) at 0° C. The reaction was allowed to be warmed up to room temperature and stirred for 3-5 hrs. The mixture was further heated at 60° C. for 2 days. The suspension was concentrated to dryness and the solid was gently washed with DCM and further dried under high vacuum to provide intermediate 13 as an off white solid (240 mg). ¹H NMR (400 MHz, DMSO-d₆) δ (ppm): 7.58 (d, J=8.8 Hz, 2H), 6.96 (d, J=8.8 Hz, 2H), 5.78 (s, 1H), 3.77 (s, 3H). MS (ESI): Calcd. for C10H10N2O2: 190, found 191 (M+H)⁺.

Intermediate 14

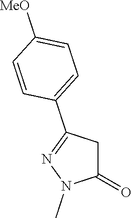

To a solution of ethyl 3-(4-methoxyphenyl)-3-oxopropanoate (0.67 mL, 3.5 mmol) in anhydrous ethanol (5.0 mL) was added dropwise a solution of 1-methyl hydrazine (0.18 mL, 3.5 mmol) at 0° C. The reaction was allowed to be warmed up to room temperature and stirred for 3-5 hrs. The mixture was further heated at 60° C. for 2 days. The suspension was concentrated to dryness and the solid was gently washed with DCM and further dried under high vacuum to provide intermediate 14 as an off white solid (470 mg). ¹H NMR (400 MHz, DMSO-d₆) δ (ppm): 10.94 (bs, 1H), 7.60 (d, J=8.4 Hz, 2H), 6.90 (d, J=8.4 Hz, 2H), 5.68 (s, 1H), 3.76 (s, 3H), 3.53 (s, 3H). MS (ESI): Calcd. for C11H12N2O2: 204, found 205 (M+H)⁺.

Intermediate 15

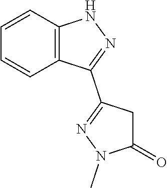 [SMILES: CN1N=C(c2n[nH]c3ccccc23)CC1=O]

To a solution of ethyl 3-(1H-indazol-3-yl)-3-oxopropanoate (1 g, 4.31 mmol) in anhydrous ethanol (70 mL) was added dropwise a solution of methylhydrazine (0.023 mL, 4.31 mmol) in THF (1 N) at 0° C. The reaction was allowed to be warmed up to room temperature and stirred for 28 hrs. The mixture was further heated under reflux for 8 hrs. Reaction mixture was concentrated on rotavapor to dryness to provide intermediate 15 as an orange solid (922 mg, 100%). ¹H NMR (400 MHz, DMSO-d₆) δ (ppm): 7.72 (d, J=8.0 Hz, 1H), 6.95 (d, J=8.4 Hz, 1H), 6.80 (t, J=7.2 Hz, 1H), 6.59 (t, J=7.2 Hz, 1H), 5.20 (brs, 1H), 3.07 (s, 3H), 3.00 (s, 2H). MS (ESI): Calcd for C11H11N4O: 215.09, found: 215.09 (M+H)⁺.

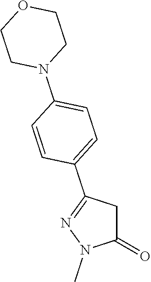 [SMILES: CN1N=C(c2ccc(N3CCOCC3)cc2)CC1=O]

Intermediate 16

To a solution of ethyl 3-(4-morpholinophenyl)-3-oxopropanoate (498 mg, 1.8 mmol) in anhydrous ethanol (10 mL) was added dropwise a solution of methylhydrazine (0.09 mL, 1.8 mmol) in THF (1 N) at 0° C. The reaction was allowed to be warmed up to room temperature and stirred for 32 hrs. The mixture was further heated under reflux for 8 hrs. Reaction mixture was concentrated on rotavapor to dryness to provide intermediate 16 as an orange solid (465 mg, 100%). ¹H NMR (400 MHz, DMSO-d₆) δ (ppm): 7.78 (d, J=9.2 Hz, 2H), 6.96 (d, J=9.2 Hz, 2H), 3.99 (s, 2H), 3.71 (t, J=5.2 Hz, 4H), 3.31 (s, 3H), 3.29 (t, J=5.2 Hz, 4H), MS (ESI): Calcd for C14H18N3O2: 260.1, found: 260.2 (M+H)⁺.

Intermediate 17

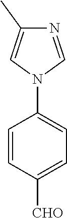

A mixture of 4-fluorobenzaldehyde (1.76 g, 14.18 mmol) and 4-methylimidazole (2.91 g, 35.45 mmol) in acetonitrile (80 mL) was charged with DIEA (6.18 mL, 35.45 mmol). The solution was refluxed for 72 hrs., upon which TLC indicated still 30% starting material was left. The mixture was concentrated on rotavapor to dryness and the resulting crude product was purified by Teledyne-Isco flash system by using CH₂Cl₂/MeOH, 0 to 8% of methanol in dichloromethane to provide intermediate 17 as a viscous solid (700 mg). ¹H NMR (400 MHz, DMSO-d₆) δ (ppm): 10.62 (s, 0.48H), 8.96 (s, 0.45H), 8.63 (m, 2H), 8.46 (m, 2H), 8.09 (s, 1H), 7.30 (s, 1H), 2.79+2.74 (s, 3H). MS (ESI): Calcd for C11H10N2O: 186, found: 187(M+H)⁺.

Intermediate 18

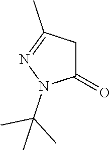

To a solution of ethyl acetoacetate (0.45 mL, 3.5 mmol) in anhydrous ethanol (5.0 mL) was added dropwise a solution of t-butylhydrazine (0.45 mL, 3.5 mmol) at 0° C. The reaction was allowed to be warmed up to room temperature and stirred for 3-5 hrs. The mixture was further heated at 60° C. overnight. The suspension was concentrated to dryness and the resulting crude product was purified by Teledyne-Isco flash system by using CH₂Cl₂/MeOH, 0 to 5% of methanol in dichloromethane to provide intermediate 18 as a white solid (260 mg). ¹H NMR (400 MHz, DMSO-d₆) δ (ppm): 5.78 (s, 1H), 2.28 (s, 3H), 1.60 (s, 9H); MS (ESI): Calcd. for C8H14N2O: 154, found 155 (M+H)⁺.

Intermediate 19

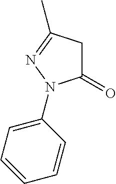

To a solution of ethyl acetoacetate (0.45 mL, 3.5 mmol) in anhydrous ethanol (5.0 mL) was added dropwise a solution of phenylhydrazine (0.35 mL, 3.5 mmol) at 0° C. The reaction was allowed to be warmed up to room temperature and stirred for 3-5 hrs. The mixture was further heated at 60° C. overnight. The suspension was concentrated to dryness and the resulting crude product was purified by Teledyne-Isco flash system by using CH₂Cl₂/MeOH, 0 to 5% of methanol in dichloromethane to provide intermediate 19 as an off white solid (270 mg). ¹H NMR (400 MHz, DMSO-d₆) δ (ppm): 11.45 (bs, 1H), 7.82-7.17 (m, 5H), 5.36 (bs, 1H), 2.11 (s, 3H); MS (ESI): Calcd. for C10H10N2O: 174, found 175 (M+H)⁺.

Intermediate 20

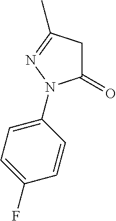

To a solution of ethyl acetoacetate (0.45 mL, 3.5 mmol) in anhydrous ethanol (5.0 mL) was added dropwise a solution of 4-fluorophenylhydrazine (0.57 mL, 3.5 mmol) at 0° C. The reaction was allowed to be warmed up to room temperature and stirred for 3-5 hrs. The mixture was further heated at 60° C. overnight. The suspension was concentrated to dryness and the resulting crude product was purified by Teledyne-Isco flash system by using CH₂Cl₂/MeOH, 0 to 5% of methanol in dichloromethane to provide intermediate 20 as a light yellow solid (190 mg). ¹H NMR (400 MHz, DMSO-d₆) δ (ppm): 7.70-7.67 (m, 2H), 7.23 (t, 2H), 5.33 (s, 1H), 2.08 (s, 3H); MS (ESI): Calcd. for C10H9FN2O: 192, found 193 (M+H)⁺.

Intermediate 21

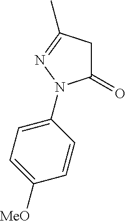

To a solution of ethyl acetoacetate (0.45 mL, 3.5 mmol) in anhydrous ethanol (5.0 mL) was added dropwise a solution of 4-methoxyphenylhydrazine (0.61 mL, 3.5 mmol) at 0° C. The reaction was allowed to be warmed up to room temperature and stirred for 3-5 hrs. The mixture was further heated at 60° C. overnight. The suspension was concentrated to dryness and the resulting crude product was purified by Teledyne-Isco flash system by using CH₂Cl₂/MeOH, 0 to 5% of methanol in dichloromethane to provide intermediate 21 as a light yellow solid (320 mg). ¹H NMR (400 MHz, DMSO-d₆) δ (ppm): 12.00 (bs, 1H), 7.58-7.07 (m, 4H), 5.78 (s, 1H), 3.80 (s, 3H), 2.28 (s, 3H); MS (ESI): Calcd. for C11H12N2O2: 204, found 205 (M+H)⁺.

Intermediate 22

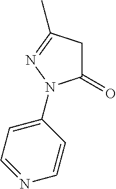

To a solution of ethyl acetoacetate (0.45 mL, 3.5 mmol) in anhydrous ethanol (5.0 mL) was added dropwise a solution of 4-pyridylhydrazine (0.35 mL, 3.5 mmol) at 0° C. The reaction was allowed to be warmed up to room temperature and stirred for 3-5 hrs. The mixture was further heated at 60° C. overnight. The suspension was concentrated to dryness and the resulting crude product was purified by Teledyne-Isco flash system by using CH₂Cl₂/MeOH, 0 to 5% of methanol in dichloromethane to provide intermediate 22 as a light yellow solid (130 mg). ¹H NMR (400 MHz, DMSO-d₆) δ (ppm): 8.78 (d, J=6.8 Hz, 2H), 6.90 (d, J=6.4 Hz, 2H), 5.52 (s, 1H), 2.19 (s, 3H). MS (ESI): Calcd. for C9H9N3O: 175, found 176 (M+H)⁺.

Intermediate 23

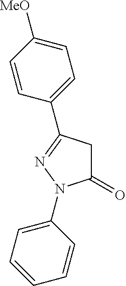 [SMILES: COc1ccc(C2=NN(c3ccccc3)C(=O)C2)cc1]

To a solution of ethyl 3-(4-methoxyphenyl)-3-oxopropanoate (0.67 mL, 3.5 mmol) in anhydrous ethanol (5.0 mL) was added dropwise a solution of 1-phenyl hydrazine (0.35 mL, 3.5 mmol) at 0° C. The reaction was allowed to be warmed up to room temperature and stirred for 3-5 hrs. The mixture was further heated at 60° C. for 2 days. The suspension was concentrated to dryness and the solid was gently washed with DCM and further dried under high vacuum to provide intermediate 23 as a white solid (670 mg). ¹H NMR (400 MHz, DMSO-d₆) δ (ppm): 11.75 (bs, 1H), 8.82-8.74 (m, 4H), 7.49-7.45 (m, 2H), 7.28-7.26 (m, 1H), 6.97 (d, J=8.0 Hz, 2H), 5.93 (s, 1H), 3.78 (s, 3H). MS (ESI): Calcd. for C16H14N2O2: 266, found 267 (M+H)⁺.

### Preparation of Exemplary Compounds

### Example 1

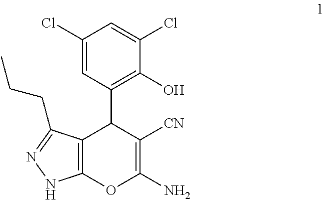 [SMILES: CCCc1n[nH]c2c1C(c1cc(Cl)cc(Cl)c1O)C(C#N)=C(N)O2]

A mixture of malonitrile (355.2 mg, 5.38 mmol) and 3,5-dichloro-2-hydroxybenzaldehyde (1.03 g, 5.38 mmol) in 10 mL of anhydrous ethanol was charged with N-methylmorpholine (0.59 mL, 5.38 mmol) for 2 min. To the mixture was added 3-propyl-1H-pyrazol-5(4H)-one (678 mg, 5.38 mmol) in one portion at room temperature. The reaction mixture was stirred at room temperature for 48 hrs. The suspension was filtered under vacuum and a pink solid was obtained. The solid was gently washed with hexanes (20 mL) and chilled ethanol (10 mL) and further dried under high vacuum to provide compound 1 as a light pinkish solid (804 mg, 41%). ¹H NMR (400 MHz, DMSO-d₆) δ (ppm): 7.50 (d, J=1.6 Hz, 1H), 6.91 (d, J=0.8 Hz, 1H), 6.91 (s, 2H), 4.66 (s, 1H), 2.40-2.37 (m, 2H), 1.47-1.43 (m, 2H), 0.81 (t, J=6.8 Hz, 3H). MS (ESI): Calcd for C16H14C12N4O2: 364, found: 365 (M+H)⁺.

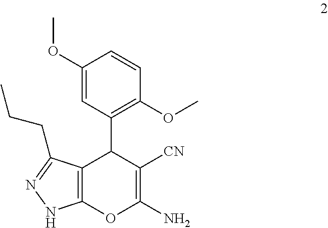 [SMILES: CCCc1n[nH]c2c1C(c1cc(OC)ccc1OC)C(C#N)=C(N)O2]

### Example 2

A mixture of malonitrile (491 mg, 7.43 mmol) and 2,5-dimethoxybenzaldehyde (1.23 g, 7.43 mmol) in 10 mL of anhydrous ethanol was charged with N-methylmorpholine (0.82 mL, 7.43 mmol) for 2 min. To the mixture was added 3-propyl-1H-pyrazol-5(4H)-one (936 mg, 7.43 mmol) in one portion at room temperature. The reaction mixture was stirred at room temperature for 48 hrs. The suspension was filtered under vacuum and a pink solid was obtained. The solid was gently washed with hexanes (20 mL) and chilled ethanol (20 mL) and further dried under high vacuum to provide compound 2 as a light yellow solid (600 mg, 24%). ¹H NMR (400 MHz, DMSO-d₆) δ (ppm): 12.00 (s, 1H), 6.91 (d, J=8.8 Hz, 1H), 6.77 (s, 2H), 6.76 (d, J=8.6 Hz, 1H), 6.49 (d, J=2.4 Hz, 1H), 4.90 (s, 1H), 3.69 (s, 3H), 3.62 (d, J=0.8 Hz, 3H), 2.30-2.02 (m, 2H), 1.42-1.02 (m, 2H), 0.61 (t, J=7.6 Hz, 3H). MS (ESI): Calcd for C18H2ON4O3: 340, found: 341 (M+H)⁺.

### Example 3

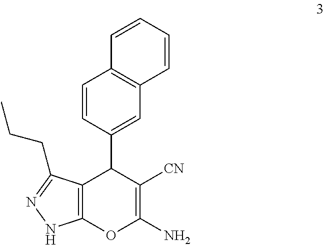 [SMILES: CCCc1n[nH]c2c1C(c1ccc3ccccc3c1)C(C#N)=C(N)O2]

A mixture of malonitrile (165 mg, 2.49 mmol) and 2-naphthaldehyde (389 mg, 2.49 mmol) in 5 mL of anhydrous ethanol was charged with N-methylmorpholine (0.27 mL, 2.49 mmol) for 2 min. To the mixture was added 3-propyl-1H-pyrazol-5(4H)-one (314 mg, 2.49 mmol) in one portion at room temperature. The reaction mixture was stirred at room temperature for 28 hrs. The suspension was filtered under vacuum and a pink solid was obtained. The solid was gently washed with hexanes (10 mL) and chilled ethanol (10 mL) and further dried under high vacuum to provide compound 3 as a light yellow solid (200 mg, 24%). ¹H NMR (400 MHz, DMSO-d₆) δ (ppm): 12.09 (s, 1H), 7.88-7.81 (m, 3H), 7.73 (s, 1H), 7.47-7.44 (m, 2H), 7.19 (d, J=8.4 Hz, 1H), 6.88 (s, 2H), 4.74 (s, 1H), 2.20-1.85 (m, 2H), 1.25-1.05 (m, 2H), 0.49 (t, J=7.2 Hz, 3H). MS (ESI): Calcd for C20H18N4O: 330, found: 331 (M+H)⁺.

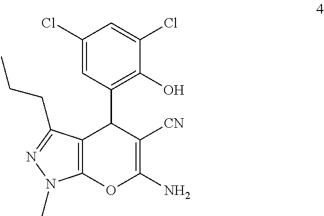 [SMILES: CCCc1nn(C)c2c1C(c1cc(Cl)cc(Cl)c1O)C(C#N)=C(N)O2]

### Example 4

A mixture of malonitrile (355 mg, 5.38 mmol) and 3,5-dichloro-2-hydroxybenzaldehyde (1.03 g, 5.38 mmol) in 10 mL of anhydrous ethanol was charged with N-methylmorpholine (0.59 mL, 5.38 mmol) for 2 min. To the mixture was added 1-methyl-3-propyl-1H-pyrazol-5(4H)-one (753 mg, 5.38 mmol) in one portion at room temperature. The reaction mixture was stirred at room temperature for 48 hrs. The suspension was filtered under vacuum and an off white solid was obtained. The solid was gently washed with hexanes (20 mL) and chilled ethanol (10 mL) and further dried under high vacuum to provide compound 4 as a light pinkish (790 mg, 39%). ¹H NMR (400 MHz, DMSO-d₆) δ (ppm): 7.33 (dd, J=2.4, 0.8 Hz, 1H), 6.99 (dd, J=2.4, 1.0 Hz, 1H), 4.78 (d, J=0.8 Hz, 1H), 3.39 (s, 3H), 2.58-2.34 (m, 2H), 1.60-1.40 (m, 2H), 0.88 (t, J=7.2 Hz, 3H). MS (ESI): Calcd for C17H16C12N4O2: 379, found 380 (M+H)⁺.

### Example 5

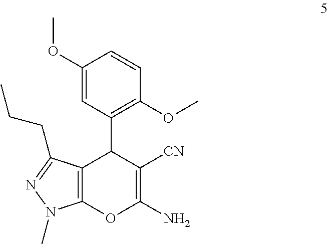 [SMILES: CCCc1nn(C)c2c1C(c1cc(OC)ccc1OC)C(C#N)=C(N)O2]

A mixture of malonitrile (491 mg, 7.43 mmol) and 2,5-dimethoxybenzaldehyde (1.23 g, 7.43 mmol) in 10 mL of anhydrous ethanol was charged with N-methylmorpholine (0.82 mL, 7.43 mmol) for 2 min. To the mixture was added 1-methyl-3-propyl-1H-pyrazol-5(4H)-one (1.03 g, 7.43 mmol) in one portion at room temperature. The reaction mixture was stirred at room temperature for 48 hrs. The suspension was filtered under vacuum and a pink solid was obtained. The solid was gently washed with hexanes (20 mL) and chilled ethanol (20 mL) and further dried under high vacuum to provide compound 5 as a light pink solid (550 mg, 21%). ¹H NMR (400 MHz, DMSO-d₆) δ (ppm): 6.94-6.89 (m, 3H), 6.72 (d, J=6.4 Hz, 1H), 6.40 (s, 1H), 4.89 (s, 1H), 3.69 (s, 3H), 3.60 (s, 3H), 3.56 (s, 3H), 2.10-1.80 (m, 2H), 1.30-1.15 (m, 2H), 0.63 (t, J=7.6 HZ, 3H). MS (ESI): Calcd for C19H22N4O3: 354, found: 355 (M+H)⁺.

### Example 6

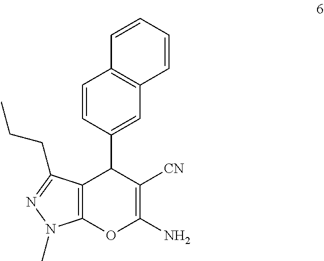 [SMILES: CCCc1nn(C)c2c1C(c1ccc3ccccc3c1)C(C#N)=C(N)O2]

A mixture of malonitrile (165 mg, 2.49 mmol) and 2-naphthaldehyde (389 mg, 2.49 mmol) in 5 mL of anhydrous ethanol was charged with N-methylmorpholine (0.27 mL, 2.49 mmol) for 2 min. To the mixture was added 1-methyl-3-propyl-1H-pyrazol-5(4H)-one (348 mg, 2.49 mmol) in one portion at room temperature. The reaction mixture was stirred at room temperature for 28 hrs. The suspension was filtered under vacuum and a pink solid was obtained. The solid was gently washed with hexanes (10 mL) and chilled ethanol (10 mL) and further dried under high vacuum to provide compound 6 as an off white powder (340 mg, 40%). ¹H NMR (400 MHz, DMSO-d₆) δ (ppm): 7.85-7.79 (m, 2H), 7.74 (s, 1H), 7.47 (dd, J=2.2, 3.4 Hz, 2H), 7.25 (d, J=1.2 Hz, 1H), 4.78 (s, 1H), 3.72 (s, 3H), 2.18-1.90 (m, 2H), 1.38-1.05 (m, 2H), 0.61 (t, J=7.6 Hz, 3H). MS (ESI): Calcd for C21H20N4O: 344, found: 345 (M+H)⁺.

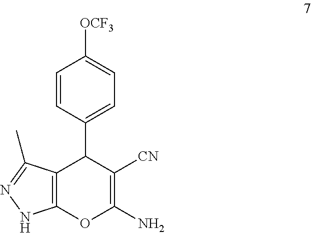 [SMILES: Cc1n[nH]c2c1C(c1ccc(OC(F)(F)F)cc1)C(C#N)=C(N)O2]

### Example 7

A mixture of malonitrile (69 mg, 1.04 mmol) and 4-(trifluoromethoxy)benzaldehyde (198 mg, 1.04 mmol) in 5 mL of anhydrous ethanol was charged with N-methylmorpholine (0.11 mL, 1.04 mmol) for 5 min. To the mixture was added 3-methyl-1H-pyrazol-5(4H)-one (102 mg, 1.04 mmol) in one portion at room temperature. The reaction mixture was stirred at room temperature for 48 hrs. The suspension was filtered under vacuum and a pink solid was obtained. The solid was gently washed with hexanes (20 mL) and chilled ethanol (5 mL) and further dried under high vacuum to provide compound 7 as an off-white powder (80 mg, 24%). ¹H NMR (400 MHz, DMSO-d₆) δ (ppm): 12.11 (s, 1H), 7.28 (d, J=1.6 Hz, 4H), 6.91 (s, 2H), 4.66 (s, 1H), 1.77 (s, 3H). MS (ESI): Calcd for C15H11F3N4O2 (M+H): 336, found: 337 (M+H)⁺.

### Example 8

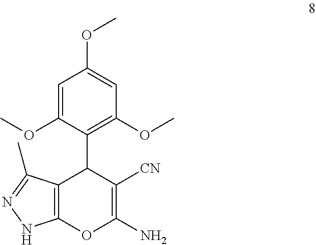

A mixture of malonitrile (90 mg, 1.36 mmol) and 2,4,6-trimethoxybenzaldehyde (268 mg, 1.36 mmol) in 6 mL of anhydrous ethanol was charged with N-methylmorpholine (151 uL, 1.36 mmol) for 10 min. To the mixture was added 3-methyl-1H-pyrazol-5(4H)-one (134 mg, 1.36 mmol) in one portion at room temperature. The reaction mixture was stirred at room temperature for 48 hrs. The suspension was filtered under vacuum and a pink solid was obtained. The solid was gently washed with hexanes (20 mL) and chilled ethanol (10 mL) and further dried under high vacuum to provide compound 8 as a bright yellow powder (220 mg, 24%). ¹H NMR (400 MHz, DMSO-d₆) δ (ppm): 7.95 (s, 1H), 6.31 (s, 2H), 3.87 (bs, 2H), 3.856 (s, 9H), 3.87-3.85 (s, 1H), 3.30 (s, 3H). MS (ESI): Calcd for C17H18N4O4 (M+H): 342, found: 343 (M+H)⁺.

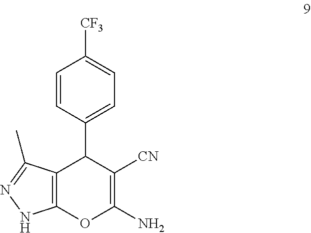 [SMILES: Cc1n[nH]c2c1C(c1ccc(C(F)(F)F)cc1)C(C#N)=C(N)O2]

### Example 9

A mixture of malonitrile (146 mg, 2.21 mmol) and 4-(trifluoromethyl)benzaldehyde (386 mg, 2.21 mmol) in 8 mL of anhydrous ethanol was charged with N-methylmorpholine (0.24 mL, 2.21 mmol) for 10 min. To the mixture was added 3-methyl-1H-pyrazol-5(4H)-one (217 mg, 2.21 mmol) in one portion at room temperature. The reaction mixture was stirred at room temperature for 48 hrs. The suspension was filtered under vacuum and a pink solid was obtained. The solid was gently washed with hexanes (20 mL) and chilled ethanol (10 mL) and further dried under high vacuum to provide compound 9 as a bright yellow powder (230 mg, 33%). ¹H NMR (400 MHz, DMSO-d₆) δ (ppm): 12.49 (s, 1H), 8.02 (d, J=8.4 Hz, 2H), 7.73 (d, J=7.6 Hz, 2H), 7.31 (s, 2H), 5.08 (s, 1H), 2.12 (s, 3H). MS (ESI): Calcd for C15H11F3N4O (M+H): 320, found: 321 (M+H)⁺.

### Example 10

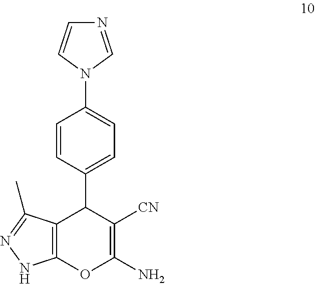 [SMILES: Cc1n[nH]c2c1C(c1ccc(-n3ccnc3)cc1)C(C#N)=C(N)O2]

A mixture of malonitrile (151 mg, 2.28 mmol) and 4-(1H-imidazol-1-yl)benzaldehyde (393 mg, 2.28 mmol) in 8 mL of anhydrous ethanol was charged with N-methylmorpholine (0.25 mL, 2.28 mmol) for 10 min. To the mixture was added 3-methyl-1H-pyrazol-5(4H)-one (224 mg, 2.28 mmol) in one portion at room temperature. The reaction mixture was stirred at room temperature for 48 hrs. The suspension was filtered under vacuum and a pink solid was obtained. The solid was gently washed with hexanes (20 mL) and chilled ethanol (10 mL) and further dried under high vacuum to provide compound 10 as a bright yellow powder (320 mg, 44%). ¹H NMR (400 MHz, DMSO-d₆) δ (ppm): 8.20 (d, J=0.8 Hz, 1H), 7.70 (d, J=1.2 Hz, 1H), 7.17-7.59 (m, 4H), 7.07 (d, J=1.2 Hz, 1H), 5.54 (d, J=11.6 Hz, 1H), 4.72 (d, J=11.6 Hz, 1H), 4.32 (bs, 1H), 2.09 (s, 3H). MS (ESI): Calcd for C₁₇H₁₄N₆O: 318, found: 319 (M+H)⁺.

### Example 11

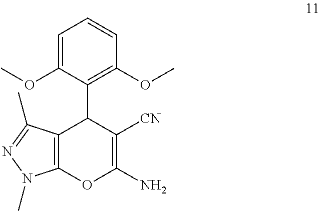

A mixture of malonitrile (93 mg, 1.41 mmol) and 2,6-dimethoxybenzaldehyde (233 mg, 1.41 mmol) in 5 mL of anhydrous ethanol was charged with N-methylmorpholine (0.15 mL, 1.41 mmol) for 10 min. To the mixture was added 1,3-dimethyl-1H-pyrazol-5(4H)-one (158 mg, 1.41 mmol) in one portion at room temperature. The reaction mixture was stirred at room temperature for 48 hrs. The suspension was filtered under vacuum and a yellow solid was obtained. The solid was gently washed with hexanes (20 mL) and chilled ethanol (10 mL) and further dried under high vacuum to provide compound 11 as an off-white powder (150 mg, 33%). ¹H NMR (400 MHz, DMSO-d₆) δ (ppm): 7.51 (t, J=8.4 Hz, 1H), 7.13 (s, 1H), 7.70 (d, J=8.4 Hz, 1H), 5.28 (d, J=10.8 Hz, 1H), 4.12 (s, 1H), 4.11 (s, 6H), 4.11-4.10 (m, 1H), 3.65 (s, 3H), 2.17 (s, 3H). MS (ESI): Calcd for C17H18N4O3: 326, found: 327 (M+H)⁺.

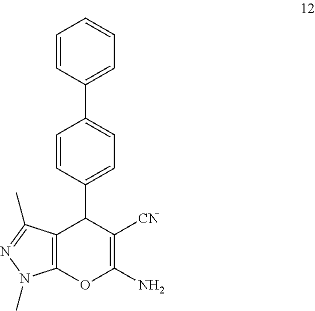 [SMILES: Cc1nn(C)c2c1C(c1ccc(-c3ccccc3)cc1)C(C#N)=C(N)O2]

### Example 12

A mixture of malonitrile (94 mg, 1.42 mmol) and [1,1′-biphenyl]-4-carbaldehyde (257 mg, 1.42 mmol) in 5 mL of anhydrous ethanol was charged with N-methylmorpholine (0.15 mL, 1.42 mmol) for 10 min. To the mixture was added 1,3-dimethyl-1H-pyrazol-5(4H)-one (158 mg, 1.42 mmol) in one portion at room temperature. The reaction mixture was stirred at room temperature for 48 hrs. The suspension was filtered under vacuum and a yellow solid was obtained. The solid was gently washed with hexanes (20 mL) and chilled ethanol (10 mL) and further dried under high vacuum to provide compound 12 as a yellow powder (250 mg, 52%). ¹H NMR (400 MHz, DMSO-d₆) δ (ppm): 7.64-7.52 (m, 4H), 7.43 (t, J=7.6 Hz, 2H), 7.33 (d, J=7.2 Hz, 1H), 7.24 (d, J=8.0 Hz, 2H), 7.07 (s, 2H), 4.61 (s, 1H), 3.59 (s, 3H), 1.69 (s, 3H). MS (ESI): Calcd for C21H18N4O: 342. found: 343 (M+H)⁺.

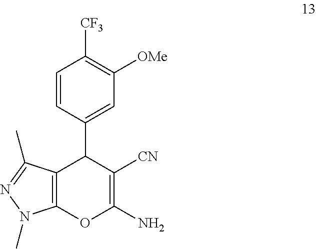 [SMILES: COc1cc(C2C(C#N)=C(N)Oc3c2c(C)nn3C)ccc1C(F)(F)F]

### Example 13

A mixture of malonitrile (97 mg, 1.47 mmol) and 3-methoxy-4-(trifluoromethyl)benzaldehyde (298 mg, 1.47 mmol) in 5 mL of anhydrous ethanol was charged with N-methylmorpholine (0.16 mL, 1.47 mmol) for 10 min. To the mixture was added 1,3-dimethyl-1H-pyrazol-5(4H)-one (164 mg, 1.47 mmol) in one portion at room temperature. The reaction mixture was stirred at room temperature for 48 hrs. The suspension was filtered under vacuum and a yellow solid was obtained. The solid was gently washed with hexanes (20 mL) and chilled ethanol (10 mL) and further dried under high vacuum to provide compound 13 as a yellow powder (300 mg, 52%). ¹H NMR (400 MHz, DMSO-d₆) δ (ppm): 7.54 (d, J=8.0 Hz, 1H), 7.15 (bs, 2H), 7.13 (d, J=8.0 Hz, 1H), 6.82 (d, J=8.0 Hz, 1H), 4.70 (s, 1H), 3.84 (s, 3H), 3.58 (s, 3H), 1.70 (s, 3H). MS (ESI): Calcd for C17H15F3N4O2: 364, found: 365 (M+H)⁺.

### Example 14

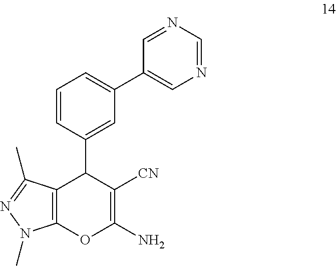 [SMILES: Cc1nn(C)c2c1C(c1cccc(-c3cncnc3)c1)C(C#N)=C(N)O2]

A mixture of malonitrile (100 mg, 1.51 mmol) and 3-(Pyrimidin-5-yl)benzaldehyde (278 mg, 1.51 mmol) in 5 mL of anhydrous ethanol was charged with N-methylmorpholine (0.17 mL, 1.51 mmol) for 10 min. To the mixture was added 1,3-dimethyl-1H-pyrazol-5(4H)-one (169 mg, 1.51 mmol) in one portion at room temperature. The reaction mixture was stirred at room temperature for 48 hrs. The suspension was filtered under vacuum and a yellow solid was obtained. The solid was gently washed with hexanes (20 mL) and chilled ethanol (10 mL) and further dried under high vacuum to provide compound 14 as a yellow powder (255 mg, 49%). ¹H NMR (400 MHz, DMSO-d₆) δ (ppm): 9.20 (s, 1H), 9.09 (s, 2H), 7.69 (t, J=7.2 Hz, 2H), 7.48 (t, J=7.6 Hz, 1H), 7.24 (d, J=7.6 Hz, 1H), 7.09 (bs, 2H), 4.69 (s, 1H), 3.58 (s, 3H), 1.69 (s, 3H). MS (ESI): Calcd for C19H16N6O: 344, found: 345 (M+H)⁺.

### Example 15

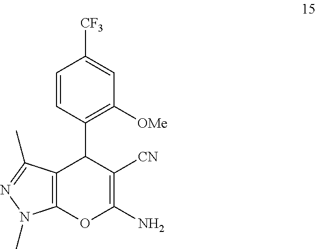 [SMILES: COc1cc(C(F)(F)F)ccc1C1C(C#N)=C(N)Oc2c1c(C)nn2C]

A mixture of malonitrile (101 mg, 1.53 mmol) and 2-methoxy-4-(trifluoromethyl)benzaldehyde (313 mg, 1.53 mmol) in 5 mL of anhydrous ethanol was charged with N-methylmorpholine (0.17 mL, 1.53 mmol) for 10 min. To the mixture was added 1,3-dimethyl-1H-pyrazol-5(4H)-one (172 mg, 1.53 mmol) in one portion at room temperature. The reaction mixture was stirred at room temperature for 48 hrs. The suspension was filtered under vacuum and a yellow solid was obtained. The solid was gently washed with hexanes (20 mL) and chilled ethanol (10 mL) and further dried under high vacuum to provide compound 15 as a yellow powder (150 mg, 58%). ¹H NMR (400 MHz, DMSO-d₆) δ (ppm): 7.28 (s, 1H), 7.24 (s, 1H), 7.20 (m, 1H), 7.07 (brs, 2H), 5.01 (s, 1H), 3.86 (s, 3H), 3.29 (s, 3H), 1.64 (s, 3H). MS (ESI): Calcd for C17H15F3N4O2: 364, found: 365 (M+H)⁺.

### Example 16

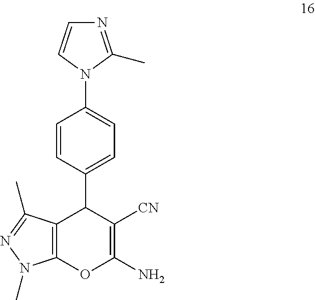 [SMILES: Cc1nn(C)c2c1C(c1ccc(-n3ccnc3C)cc1)C(C#N)=C(N)O2]

A mixture of malonitrile (110 mg, 1.66 mmol) and 4-(2-methyl-1H-imidazol-1-yl)benzaldehyde (309 mg, 1.66 mmol) in 8 mL of anhydrous ethanol was charged with N-methylmorpholine (0.18 mL, 1.66 mmol) for 10 min. To the mixture was added 1,3-dimethyl-1H-pyrazol-5(4H)-one (186 mg, 1.66 mmol) in one portion at room temperature. The reaction mixture was stirred at room temperature for 48 hrs. The suspension was filtered under vacuum and a pink solid was obtained. The solid was gently washed with hexanes (20 mL) and chilled ethanol (10 mL) and further dried under high vacuum to provide compound 16 as a bright yellow powder (260 mg, 44%). ¹H NMR (400 MHz, DMSO-d₆) δ (ppm): 7.74 (d, J=8.4 Hz, 2H), 7.67 (d, J=8.4 Hz, 2H), 7.63 (s, 1H), 7.49 (brs, 2H), 7.24 (s, 1H), 5.05 (s, 1H), 3.96 (s, 3H), 2.61 (s, 3H), 2.07 (s, 3H). MS (ESI): Calcd for C19H18N6O: 346, found: 347 (M+H)⁺.

### Example 17

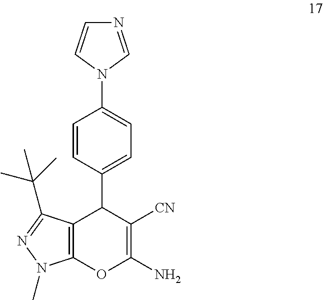 [SMILES: Cn1nc(C(C)(C)C)c2c1OC(N)=C(C#N)C2c1ccc(-n2ccnc2)cc1]

A mixture of malonitrile (171.0 mg, 2.59 mmol) and 4-(1H-imidazol-1-yl)benzaldehyde (446.0 mg, 2.59 mmol) in anhydrous ethanol (10.0 mL) was charged with N-methylmorpholine (0.3 mL, 2.59 mmol) for 2 minutes. To the mixture was added 3-(tert-butyl)-1-methyl-1H-pyrazol-5(4H)-one (400 mg, 2.59 mmol) in one portion at room temperature. The reaction mixture was stirred at room temperature for 48 hrs. The suspension was filtered under vacuum and off white solid was obtained. The solid was gently washed with hexanes (20 mL) and chilled ethanol (10 mL) and further dried under high vacuum to provide compound 17 as an off white solid (800 mg, 82%). ¹H NMR (400 MHz, DMSO-d₆) δ (ppm): 8.23 (s, 1H), 7.72 (s, 1H), 7.59 (d, J=8.0 Hz, 2H), 7.24 (d, J=8.0 Hz, 2H), 7.09 (s, 1H), 7.02 (s, 2H), 4.73 (s, 1H), 3.66 (s, 2H), 0.95 (s, 9H). MS (ESI): Calcd for C21H22N6O: 374, found: 375(M+H)⁺.

### Example 18

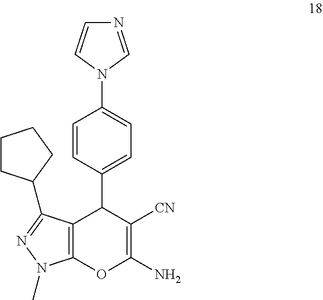 [SMILES: Cn1nc(C2CCCC2)c2c1OC(N)=C(C#N)C2c1ccc(-n2ccnc2)cc1]

A mixture of malonitrile (60.0 mg, 0.9 mmol) and 4-(1H-imidazol-1-yl)benzaldehyde (155.0 mg, 0.9 mmol) in anhydrous ethanol (4.0 mL) was charged with N-methylmorpholine (0.1 mL, 0.9 mmol) for 2 minutes. To the mixture was added 3-cyclopentyl-1-methyl-1H-pyrazol-5(4H)-one (150.0 mg, 0.9 mmol) in one portion at room temperature. The reaction mixture was stirred at room temperature for 48 hrs. The suspension was filtered under vacuum and off white solid was obtained. The solid was gently washed with hexanes (20 mL) and chilled ethanol (10 mL) and further dried under high vacuum to provide compound 18 as an off white solid (246 mg, 70%). ¹H NMR (400 MHz, DMSO-d₆) δ (ppm): 8.24 (s, 1H), 7.73 (s, 1H), 7.59 (d, J=8.0 Hz, 2H), 7.30 (d, J=8.0 Hz, 2H), 7.10-7.08 (d, J=8.0 Hz, 3H), 4.67 (s, 1H), 3.63 (s, 3H), 2.48 (m, 1H), 1.76-1.28 (m, 8H). MS (ESI): Calcd for C25H18N6O2: 386, found: 387 (M+H)⁺.

### Example 19

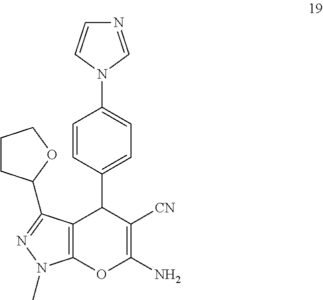 [SMILES: Cn1nc(C2CCCO2)c2c1OC(N)=C(C#N)C2c1ccc(-n2ccnc2)cc1]

A mixture of malonitrile (60.0 mg, 0.9 mmol) and 4-(1H-imidazol-1-yl)benzaldehyde (155.0 mg, 0.9 mmol) in anhydrous ethanol (4.0 mL) was charged with N-methylmorpholine (0.1 mL, 0.9 mmol) for 2 minutes. To the mixture was added 3-cyclopentyl-1-methyl-1H-pyrazol-5(4H)-one (150.0 mg, 0.9 mmol) in one portion at room temperature. The reaction mixture was stirred at room temperature for 48 hrs. The suspension was filtered under vacuum and off white solid was obtained. The solid was gently washed with hexanes (20 mL) and chilled ethanol (10 mL) and further dried under high vacuum to provide compound 19 as an off white solid (246 mg, 70%). ¹H NMR (400 MHz, DMSO-d₆) δ (ppm): 8.24 (s, 1H), 7.73 (s, 1H), 7.59 (d, J=8.0 Hz, 2H), 7.30 (d, J=8.0 Hz, 2H), 7.10-7.08 (d, J=8.0 Hz, 3H), 4.67 (s, 1H), 3.63 (s, 3H), 2.48 (m, 1H), 1.76-1.28 (m, 8H). MS (ESI): Calcd for C25H18N6O2: 386, found: 387 (M+H)⁺.

### Example 20

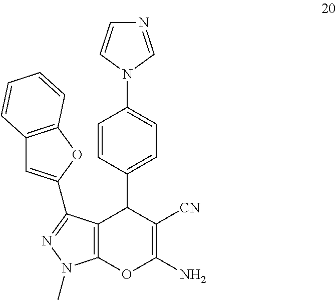 [SMILES: Cn1nc(-c2cc3ccccc3o2)c2c1OC(N)=C(C#N)C2c1ccc(-n2ccnc2)cc1]

A mixture of malonitrile (60.0 mg, 0.9 mmol) and 4-(1H-imidazol-1-yl)benzaldehyde (155.0 mg, 0.9 mmol) in anhydrous ethanol (4.0 mL) was charged with N-methylmorpholine (0.1 mL, 0.9 mmol) for 2 minutes. To the mixture was added 3-(benzofuran-2-yl)-1-methyl-1H-pyrazol-5(4H)-one (193.0 mg, 0.9 mmol) in one portion at room temperature. The reaction mixture was stirred at room temperature for 48 hrs. The suspension was filtered under vacuum and off white solid was obtained. The solid was gently washed with hexanes (20 mL) and chilled ethanol (10 mL) and further dried under high vacuum to provide compound 20 as an off white solid (255 mg, 65%). ¹H NMR (400 MHz, DMSO-d₆) δ (ppm): 8.16 (s, 1H), 7.65 (s, 1H), 7.55-7.52 (m, 4H), 7.41 (d, J=8.0 Hz, 2H), 7.28-7.7.16 (m, 4H), 7.04 (s, 1H), 6.85 (s, 1H), 5.05 (s, 1H), 3.83 (s, 3H). MS (ESI): Calcd for C25H18N6O2: 434, found: 435 (M+H)⁺.

### Example 21

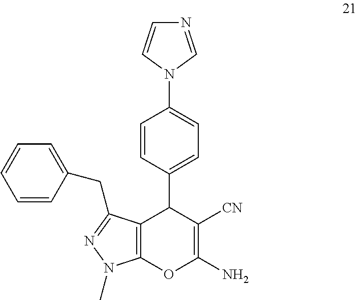 [SMILES: Cn1nc(Cc2ccccc2)c2c1OC(N)=C(C#N)C2c1ccc(-n2ccnc2)cc1]

A mixture of malonitrile (60.0 mg, 0.9 mmol) and 4-(1H-imidazol-1-yl)benzaldehyde (155.0 mg, 0.9 mmol) in anhydrous ethanol (4.0 mL) was charged with N-methylmorpholine (0.1 mL, 0.9 mmol) for 2 minutes. To the mixture was added 3-benzyl-1-methyl-1H-pyrazol-5(4H)-one (170.0 mg, 0.9 mmol) in one portion at room temperature. The reaction mixture was stirred at room temperature for 48 hrs. The suspension was filtered under vacuum and off white solid was obtained. The solid was gently washed with hexanes (20 mL) and chilled ethanol (10 mL) and further dried under high vacuum to provide compound 21 as an off white solid (330 mg, 89%). ¹H NMR (400 MHz, DMSO-d₆) δ (ppm): 8.20 (s, 1H), 7.69 (s, 1H), 7.51 (d, J=8.0 Hz, 2H), 7.21 (d, J=8.0 Hz, 2H), 7.15-7.08 (m, 6H), 6.86 (d, J=7.2 Hz, 2H), 4.46 (s, 1H), 3.66 (s, 3H), 3.53 (d, J=15.2 Hz, 1H), 3.28 (s, 1H). MS (ESI): Calcd for C24H20N6O: 408, found: 409 (M+H)⁺.

### Example 22

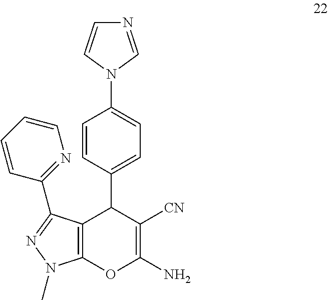 [SMILES: Cn1nc(-c2ccccn2)c2c1OC(N)=C(C#N)C2c1ccc(-n2ccnc2)cc1]

A mixture of malonitrile (60.0 mg, 0.9 mmol) and 4-(1H-imidazol-1-yl)benzaldehyde (155.0 mg, 0.9 mmol) in anhydrous ethanol (4.0 mL) was charged with N-methylmorpholine (0.1 mL, 0.9 mmol) for 2 minutes. To the mixture was added 1-methyl-3-(pyridin-2-yl)-1H-pyrazol-5(4H)-one (158.0 mg, 0.9 mmol) in one portion at room temperature. The reaction mixture was stirred at room temperature for 48 hrs. The suspension was filtered under vacuum and off white solid was obtained. The solid was gently washed with hexanes (20 mL) and chilled ethanol (10 mL) and further dried under high vacuum to provide compound 22 as an off white solid (365 mg, 99%). ¹H NMR (400 MHz, DMSO-d₆) δ (ppm): 8.46-8.45 (m, 1H), 8.13 (s, 1H), 7.78-7.63 (m, 3H), 7.44 (d, J=8.0 Hz, 2H), 7.29 (d, J=8.4 Hz, 2H), 7.20-7.17 (m, 3H), 7.04 (s, 1H), 5.13 (s, 1H), 4.34 (t, 1H), 3.81 (s, 3H), 3.47-3.41 (m, 2H), 1.06 (t, 3H). MS (ESI): Calcd for C22H17N7O: 395, found: 396 (M+H)⁺.

### Example 23

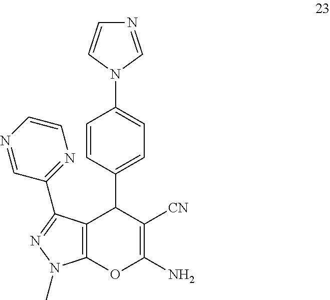 [SMILES: Cn1nc(-c2cnccn2)c2c1OC(N)=C(C#N)C2c1ccc(-n2ccnc2)cc1]

A mixture of malonitrile (60.0 mg, 0.9 mmol) and 4-(1H-imidazol-1-yl)benzaldehyde (155.0 mg, 0.9 mmol) in anhydrous ethanol (4.0 mL) was charged with N-methylmorpholine (0.1 mL, 0.9 mmol) for 2 minutes. To the mixture was added 1-methyl-3-(pyrazin-2-yl)-1H-pyrazol-5(4H)-one (159.0 mg, 0.9 mmol) in one portion at room temperature. The reaction mixture was stirred at room temperature for 48 hrs. The suspension was filtered under vacuum and off white solid was obtained. The solid was gently washed with hexanes (20 mL) and chilled ethanol (10 mL) and further dried under high vacuum to provide compound 23 as an off white solid (340 mg, 95%). ¹H NMR (400 MHz, DMSO-d₆) δ (ppm): 8.98 (s, 1H), 8.50-8.42 (m, 2H), 8.14 (s, 1H), 7.63 (s, 1H), 7.45 (d, J=8.4 Hz, 2H), 7.28 (d, J=8.4 Hz, 2H), 7.05 (s, 1H), 5.08 (s, 1H), 3.85 (s, 3H). MS (ESI): Calcd for C₂₁H₁₆N₈O: 396, found: 397 (M+H)⁺.

### Example 24

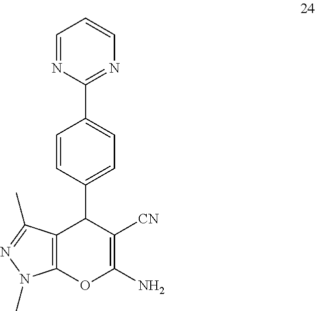 [SMILES: Cc1nn(C)c2c1C(c1ccc(-c3ncccn3)cc1)C(C#N)=C(N)O2]

A mixture of malonitrile (102 mg, 1.54 mmol) and 4-(pyrimidin-2-yl)benzaldehyde (284 mg, 1.54 mmol) in 8 mL of anhydrous ethanol was charged with N-methylmorpholine (0.17 mL, 1.54 mmol) for 10 min. To the mixture was added 1,3-dimethyl-1H-pyrazol-5(4H)-one (172 mg, 1.54 mmol) in one portion at room temperature. The reaction mixture was stirred at room temperature for 48 hrs. The suspension was filtered under vacuum and a pink solid was obtained. The solid was gently washed with hexanes (20 mL) and chilled ethanol (10 mL) and further dried under high vacuum to provide compound 24 as a bright yellow powder (200 mg, 44%). ¹H NMR (400 MHz, DMSO-d₆) δ (ppm): 9.43 (t, J=5.6 Hz, 2H), 8.87 (d, J=8.0 Hz, 2H), 7.96 (d, J=1.2 Hz, 1H), 7.86 (d, J=8.0 Hz, 2H), 7.65 (s, 2H), 5.21 (s, 1H), 4.14 (s, 3H), 2.22 (s, 3H). MS (ESI): Calcd for C19H16N6O: 344, found: 345 (M+H)⁺.

### Example 25

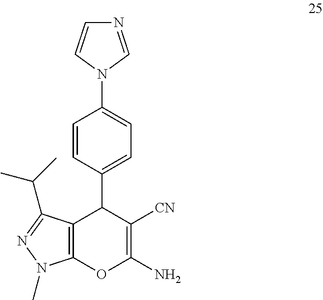 [SMILES: CC(C)c1nn(C)c2c1C(c1ccc(-n3ccnc3)cc1)C(C#N)=C(N)O2]

A mixture of malonitrile (102 mg, 1.54 mmol) and 4(1H-imidazol-1-yl)benzaldehyde (266 mg, 1.54 mmol) in 8 mL of anhydrous ethanol was charged with N-methylmorpholine (0.18 mL, 1.54 mmol) for 10 min. To the mixture was added 3-isopropyl-1-methyl-1H-pyrazol-5(4H)-one (219 mg, 1.54 mmol) in one portion at room temperature. The reaction mixture was stirred at room temperature for 48 hrs. The suspension was filtered under vacuum and a pink solid was obtained. The solid was gently washed with hexanes (20 mL) and chilled ethanol (10 mL) and further dried under high vacuum to provide compound 25 as an off white powder (230 mg, 41%). ¹H NMR (400 MHz, DMSO-d₆) δ (ppm): 8.21 (s, 1H), 7.70 (d, J=1.2 Hz, 1H), 7.57 (d, J=8.8 Hz, 2H), 7.28 (d, J=8.4 Hz, 2H), 7.06 (d, J=5.6 Hz, 3H), 4.68 (s, 1H), 3.62 (s, 3H), 2.37-2.32 (m, 1H), 0.92 (d, J=7.2 Hz, 3H), 0.77 (d, J=7.2 Hz, 3H); MS (ESI): Calcd for C20H20N6O: 360, found: 361 (M+H)⁺.

### Example 26

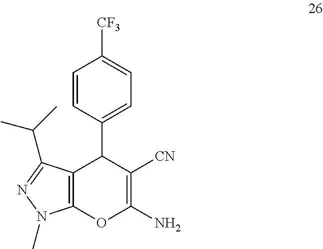 [SMILES: CC(C)c1nn(C)c2c1C(c1ccc(C(F)(F)F)cc1)C(C#N)=C(N)O2]

A mixture of malonitrile (139 mg, 2.10 mmol) and 4-(trifluoromethyl)benzaldehyde (366 mg, 2.10 mmol) in 8 mL of anhydrous ethanol was charged with N-methylmorpholine (0.23 mL, 2.10 mmol) for 10 min. To the mixture was added 3-isopropyl-1-methyl-1H-pyrazol-5(4H)-one (299 mg, 2.10 mmol) in one portion at room temperature. The reaction mixture was stirred at room temperature for 48 hrs. The suspension was filtered under vacuum and a pink solid was obtained. The solid was gently washed with hexanes (20 mL) and chilled ethanol (10 mL) and further dried under high vacuum to provide compound 26 as an off white powder (100 mg, 13%). ¹H NMR (400 MHz, DMSO-d₆) δ (ppm): 7.67 (d, J=8.0 Hz, 2H), 7.39 (d, J=8.0 Hz, 2H), 7.12 (s, 2H), 4.74 (s, 1H), 3.61 (s, 3H), 2.30 (t, J=6.8 Hz, 1H), 0.91 (d, J=7.2 Hz, 3H), 0.71 (d, J=7.2 Hz, 3H). MS (ESI): Calcd for C18H17F3N4O (M+H): 362, found: 363 (M+H)⁺.

### Example 27

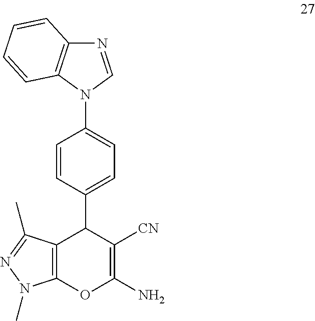 [SMILES: Cc1nn(C)c2c1C(c1ccc(-n3cnc4ccccc43)cc1)C(C#N)=C(N)O2]

A mixture of malonitrile (126 mg, 1.91 mmol) and 4-(1H-benzo[d]imidazol-1-yl)benzaldehyde (424 mg, 1.91 mmol) in 8 mL of anhydrous ethanol was charged with N-methylmorpholine (0.21 mL, 1.91 mmol) for 10 min. To the mixture was added 1,3-dimethyl-1H-pyrazol-5(4H)-one (214 mg, 1.91 mmol) in one portion at room temperature. The reaction mixture was stirred at room temperature for 48 hrs. The reaction mixture was concentrated on rotavapor to dryness and the resulting crude product was purified by Teledyne-Isco flash system by using CH₂Cl₂/MeOH, 5 to 10% of methanol in dichloromethane to provide compound 27 as a viscous solid (360 mg, 31%). ¹H NMR (400 MHz, DMSO-d₆) δ (ppm): 8.54 (d, J=3.2 Hz, 1H), 7.74-7.15 (m, 8H), 7.13 (s, 2H), 4.72 (s, 1H), 3.30 (s, 3H), 2.09 (s, 3H). MS (ESI): Calcd for C22H18N6O: 382, found: 383 (M+H)⁺.

### Example 28

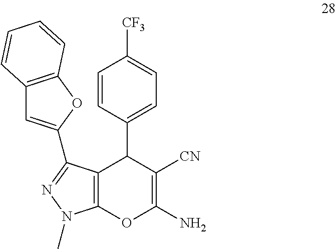 [SMILES: Cn1nc(-c2cc3ccccc3o2)c2c1OC(N)=C(C#N)C2c1ccc(C(F)(F)F)cc1]

A mixture of malonitrile (15 mg, 0.23 mmol) and 4-(trifluoromethyl)benzaldehyde (0.03 mL, 0.23 mmol) in anhydrous ethanol (1.0 mL) was charged with N-methylmorpholine (0.03 mL, 0.23 mmol) for 2 minutes. To the mixture was added 3-(benzofuran-2-yl)-1-methyl-1H-pyrazol-5(4H)-one (50 mg, 0.23 mmol) in one portion at room temperature. The reaction mixture was stirred at room temperature for 48 hrs. The suspension was filtered under vacuum and off white solid was obtained. The solid was gently washed with hexanes (20 mL) and chilled ethanol (10 mL) and further dried under high vacuum to provide compound 28 as white solid (80 mg, 89%). ¹H NMR (400 MHz, DMSO-d₆) δ (ppm): 7.64 (d, J=8.0 Hz, 2H), 7.53 (d, J=8.4 Hz, 2H), 7.48 (d, J=8.4 Hz, 2H), 7.29-7.17 (m, 4H), 6.84 (s, 1H), 5.11 (s, 1H), 3.82 (s, 3H). MS (ESI): Calcd for C23H15F3N4O2: 436, found: 437 (M+H)⁺.

### Example 29

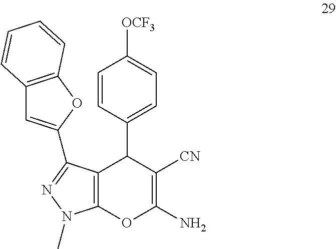 [SMILES: Cn1nc(-c2cc3ccccc3o2)c2c1OC(N)=C(C#N)C2c1ccc(OC(F)(F)F)cc1]

A mixture of malonitrile (15 mg, 0.23 mmol) and 4-(trifluoromethoxy)benzaldehyde (0.03 mL, 0.23 mmol) in anhydrous ethanol (1.0 mL) was charged with N-methylmorpholine (0.03 mL, 0.23 mmol) for 2 minutes. To the mixture was added 3-(benzofuran-2-yl)-1-methyl-1H-pyrazol-5(4H)-one (50 mg, 0.23 mmol) in one portion at room temperature. The reaction mixture was stirred at room temperature for 48 hrs. The suspension was filtered under vacuum and off white solid was obtained. The solid was gently washed with hexanes (20 mL) and chilled ethanol (10 mL) and further dried under high vacuum to provide compound 29 as white solid (75 mg, 41%). ¹H NMR (400 MHz, DMSO-d₆) δ (ppm): 7.53-7.48 (m, 2H), 7.42 (d, J=8.8 Hz, 2H), 7.28-7.17 (m, 6H), 6.80 (s, 1H), 5.04 (s, 1H), 3.82 (s, 3H). MS (ESI): Calcd for C23H15F3N4O3: 452, found: 453 (M+H)⁺.

### Example 30

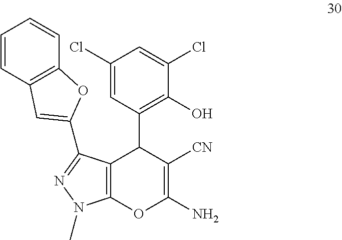 [SMILES: Cn1nc(-c2cc3ccccc3o2)c2c1OC(N)=C(C#N)C2c1cc(Cl)cc(Cl)c1O]

A mixture of malonitrile (15 mg, 0.23 mmol) and 3,5-dichloro-2-hydroxybenzaldehyde (44 mg, 0.23 mmol) in anhydrous ethanol (1.0 mL) was charged with N-methylmorpholine (0.03 mL, 0.23 mmol) for 2 minutes. To the mixture was added 3-(benzofuran-2-yl)-1-methyl-1H-pyrazol-5(4H)-one (50 mg, 0.23 mmol) in one portion at room temperature. The reaction mixture was stirred at room temperature for 48 hrs. The suspension was filtered under vacuum and off white solid was obtained. The solid was gently washed with hexanes (20 mL) and chilled ethanol (10 mL) and further dried under high vacuum to provide compound 30 as white solid (75 mg, 41%). ¹H NMR (400 MHz, DMSO-d₆) δ (ppm): 7.57-7.47 (m, 3H), 7.27-7.18 (m, 2H), 6.94-6.93 (m, 4H), 5.14 (s, 1H), 4.36-4.33 (m, 1H), 3.67 (s, 1H), 3.46-3.43 (m, 2H), 1.05 (t, 3H). MS (ESI): Calcd for C22H14C12N4O3: 452, found: 453 (M+H)⁺.

### Example 31

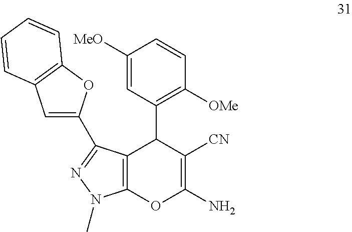 [SMILES: COc1ccc(OC)c(C2C(C#N)=C(N)Oc3c2c(-c2cc4ccccc4o2)nn3C)c1]

A mixture of malonitrile (15 mg, 0.23 mmol) and 2,5-dimethoxybenzaldehyde (38 mg, 0.23 mmol) in anhydrous ethanol (1.0 mL) was charged with N-methylmorpholine (0.03 mL, 0.23 mmol) for 2 minutes. To the mixture was added 3-(benzofuran-2-yl)-1-methyl-1H-pyrazol-5(4H)-one (50 mg, 0.23 mmol) in one portion at room temperature. The reaction mixture was stirred at room temperature for 48 hrs. The suspension was filtered under vacuum and off white solid was obtained. The solid was gently washed with hexanes (20 mL) and chilled ethanol (10 mL) and further dried under high vacuum to provide compound 31 as white solid (95 mg, 55%). ¹H NMR (400 MHz, DMSO-d₆) δ (ppm): 7.53 (d, J=7.6 Hz, 1H), 7.53 (d, J=8.0 Hz, 1H), 7.27 (t, 1H), 7.19 (t, 1H), 7.08 (s, 1H), 6.92 (d, J=9.2 Hz, 2H), 6.77 (s, 2H), 6.72-6.69 (dd, 1H), 6.05 (m, 1H), 5.22 (s, 1H), 3.79 (d, 6H), 3.59 (s, 1H), 3.48-3.41 (m, 1H). MS (ESI): Calcd for C24H2ON4O4: 428, found: 429 (M+H)⁺.

### Example 32

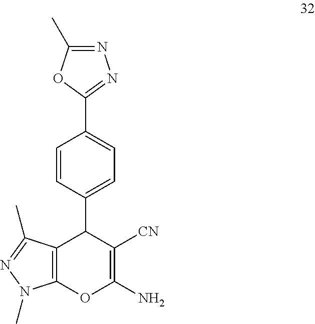 [SMILES: Cc1nnc(-c2ccc(C3C(C#N)=C(N)Oc4c3c(C)nn4C)cc2)o1]

A mixture of malonitrile (111 mg, 1.68 mmol) and 4-(5-methyl-1,3,4-oxadiazol-2-yl)benzaldehyde (316 mg, 1.68 mmol) in 8 mL of anhydrous ethanol was charged with N-methylmorpholine (0.15 mL, 1.68 mmol) for 10 min. To the mixture was added 1,3-dimethyl-1H-pyrazol-5(4H)-one (188 mg, 1.68 mmol) in one portion at room temperature. The reaction mixture was stirred at room temperature for 48 hrs. The reaction mixture was concentrated on rotavapor to dryness and the resulting crude product was purified by Teledyne-Isco flash system by using CH₂Cl₂/MeOH, 5 to 10% of methanol in dichloromethane to provide compound 32 as a light yellow solid (580 mg, 31%). ¹H NMR (400 MHz, DMSO-d₆) δ (ppm): 8.54 (d, J=3.2 Hz, 1H), 7.74-7.15 (m, 8H), 7.13 (s, 2H), 4.72 (s, 1H), 3.30 (s, 3H), 2.09 (s, 3H). MS (ESI): Calcd for C18H16N6O2: 348, found: 349 (M+H)⁺.

### Example 33

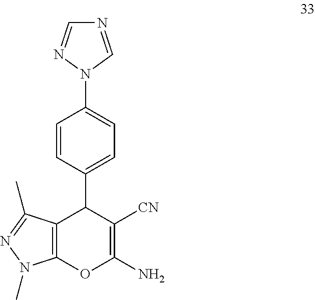 [SMILES: Cc1nn(C)c2c1C(c1ccc(-n3cncn3)cc1)C(C#N)=C(N)O2]

A mixture of malonitrile (128 mg, 1.94 mmol) and 4-(1H-1,2,4-triazol-1-yl)benzaldehyde (336 mg, 1.94 mmol) in 8 mL of anhydrous ethanol was charged with N-methylmorpholine (0.21 mL, 1.94 mmol) for 10 min. To the mixture was added 1,3-dimethyl-1H-pyrazol-5(4H)-one (214 mg, 1.94 mmol) in one portion at room temperature. The reaction mixture was stirred at room temperature for 48 hrs. The reaction mixture was concentrated on rotavapor to dryness and the resulting crude product was purified by Teledyne-Isco flash system by using CH₂Cl₂/MeOH, 5 to 10% of methanol in dichloromethane to provide compound 33 as a viscous solid (500 mg, 77%). ¹H NMR (400 MHz, DMSO-d₆) δ (ppm): 8.68 (s, 1H), 7.63 (s, 1H), 7.22 (d, J=8.0 Hz, 2H), 6.78 (d, J=8.4 Hz, 2H), 6.54 (s, 1H), 4.10 (s, 1H), 3.02 (s, 3H), 2.74 (s, 2H), 1.11 (s, 3H). MS (ESI): Calcd for C17H15N7O: 333, found: 334 (M+H)⁺.

### Example 34

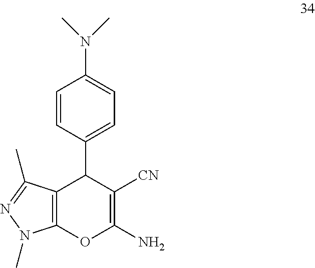 [SMILES: Cc1nn(C)c2c1C(c1ccc(N(C)C)cc1)C(C#N)=C(N)O2]

A mixture of malonitrile (60 mg, 0.91 mmol) and 4-(dimethylamino)benzaldehyde (134 mg, 0.91 mmol) in anhydrous ethanol (4.0 mL) was charged with N-methylmorpholine (0.1 mL, 0.91 mmol) for 2 minutes. To the mixture was added 1,3-dimethyl-1H-pyrazol-5(4H)-one (100 mg, 0.91 mmol) in one portion at room temperature. The reaction mixture was stirred at room temperature for 48 hrs. The suspension was concentrated to dryness and the resulting crude product was purified by Teledyne-Isco flash system by using CH₂Cl₂/MeOH, 0 to 5% of methanol in dichloromethane to provide compound 34 as orange solid (10 mg, 4%). ¹H NMR (400 MHz, DMSO-d₆) δ (ppm): 6.97-6.92 (m, 4H), 6.67-6.64 (m, 2H), 4.43 (s, 1H), 3.59 (s, 3H), 2.86 (s, 6H), 1.67 (s, 3H). MS (ESI): Calcd for C17H19N5O: 309, found: 310 (M+H)⁺.

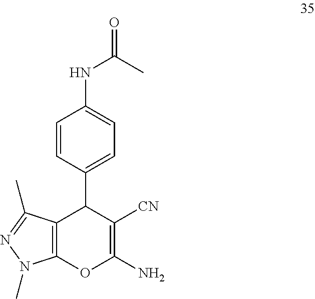 [SMILES: CC(=O)Nc1ccc(C2C(C#N)=C(N)Oc3c2c(C)nn3C)cc1]

### Example 35

A mixture of malonitrile (60 mg, 0.91 mmol) and N-(4-formylphenyl)acetamide (147 mg, 0.91 mmol) in anhydrous ethanol (4.0 mL) was charged with N-methylmorpholine (0.1 mL, 0.91 mmol) for 2 minutes. To the mixture was added 1,3-dimethyl-1H-pyrazol-5(4H)-one (100 mg, 0.91 mmol) in one portion at room temperature. The reaction mixture was stirred at room temperature for 48 hrs. The suspension was concentrated to dryness and the resulting crude product was purified by Teledyne-Isco flash system by using CH₂Cl₂/MeOH, 0 to 5% of methanol in dichloromethane to provide compound 35 as white solid (100 mg, 35%). ¹H NMR (400 MHz, DMSO-d₆) δ (ppm): 9.90 (s, 1H), 7.50 (d, J=8.4 Hz, 2H), 7.09-7.02 (m, 4H), 4.51 (s, 1H), 3.59 (s, 3H), 2.01 (s, 3H), 1.66 (s, 3H). MS (ESI): Calcd for C17H17N5O2: 323, found: 324 (M+H)⁺.

### Example 36

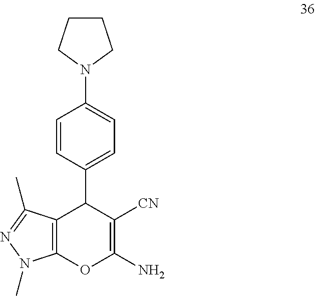 [SMILES: Cc1nn(C)c2c1C(c1ccc(N3CCCC3)cc1)C(C#N)=C(N)O2]

A mixture of malonitrile (60 mg, 0.91 mmol) and 4-(pyrrolidin-1-yl)benzaldehyde (158 mg, 0.91 mmol) in anhydrous ethanol (4.0 mL) was charged with N-methylmorpholine (0.1 mL, 0.91 mmol) for 2 minutes. To the mixture was added 1,3-dimethyl-1H-pyrazol-5(4H)-one (100 mg, 0.91 mmol) in one portion at room temperature. The reaction mixture was stirred at room temperature for 48 hrs. The suspension was concentrated to dryness and the resulting crude product was purified by Teledyne-Isco flash system by using CH₂Cl₂/MeOH, 0 to 5% of methanol in dichloromethane to provide compound 36 as light yellow solid (60 mg, 20%). ¹H NMR (400 MHz, DMSO-d₆) δ (ppm): 6.93 (m, 4H), 6.45 (d, J=8.4 Hz, 2H), 4.41 (s, 1H), 3.59 (s, 3H), 3.21-3.17 (m, 4H), 1.95-1.92 (m, 4H), 1.67 (s, 3H). MS (ESI): Calcd for C19H21N5O: 335, found: 336 (M+H)⁺.

### Example 37

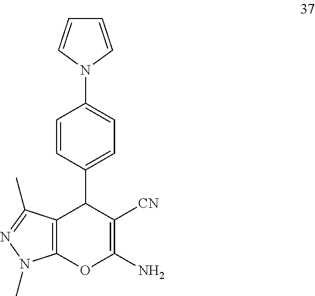 [SMILES: Cc1nn(C)c2c1C(c1ccc(-n3cccc3)cc1)C(C#N)=C(N)O2]

A mixture of malonitrile (60 mg, 0.91 mmol) and 4-(1H-pyrrol-1-yl)benzaldehyde (154 mg, 0.91 mmol) in anhydrous ethanol (4.0 mL) was charged with N-methylmorpholine (0.1 mL, 0.91 mmol) for 2 minutes. To the mixture was added 1,3-dimethyl-1H-pyrazol-5(4H)-one (100 mg, 0.91 mmol) in one portion at room temperature. The reaction mixture was stirred at room temperature for 48 hrs. The suspension was concentrated to dryness and the resulting crude product was purified by Teledyne-Isco flash system by using CH₂Cl₂/MeOH, 0 to 5% of methanol in dichloromethane to provide compound 37 as white solid (125 mg, 42%). ¹H NMR (400 MHz, DMSO-d₆) δ (ppm): 7.51 (d, J=8.8 Hz, 2H), 7.34-7.33 (m, 2H), 7.24 (d, J=8.4 Hz, 2H), 7.09 (s, 1H), 6.26-6.25 (m, 2H), 4.63 (s, 1H), 3.61 (s, 3H), 1.70 (s, 3H). MS (ESI): Calcd for C19H17N5O: 331, found: 332 (M+H)⁺.

### Example 38

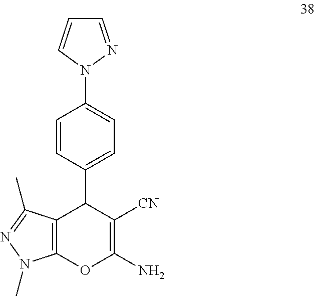 [SMILES: Cc1nn(C)c2c1C(c1ccc(-n3cccn3)cc1)C(C#N)=C(N)O2]

A mixture of malonitrile (60 mg, 0.91 mmol) and 4-(1H-pyrrol-1-yl)benzaldehyde (154 mg, 0.91 mmol) in anhydrous ethanol (4.0 mL) was charged with N-methylmorpholine (0.1 mL, 0.91 mmol) for 2 minutes. To the mixture was added 1,3-dimethyl-1H-pyrazol-5(4H)-one (100 mg, 0.91 mmol) in one portion at room temperature. The reaction mixture was stirred at room temperature for 48 hrs. The suspension was concentrated to dryness and the resulting crude product was purified by Teledyne-Isco flash system by using CH₂Cl₂/MeOH, 0 to 5% of methanol in dichloromethane to provide compound 38 as white solid (115 mg, 39%). ¹H NMR (400 MHz, DMSO-d₆) δ (ppm): 8.46 (m, 1H), 7.78 (d, J=8.0 Hz, 2H), 7.73 (s, 1H), 7.29 (d, J=8.4 Hz, 2H), 7.10 (s, 2H), 6.54-6.53 (m, 1H), 4.65 (s, 1H), 3.61 (s, 3H), 1.70 (s, 3H). MS (ESI): Calcd for C18H16N6O: 332, found: 333(M+H)⁺.

### Example 39

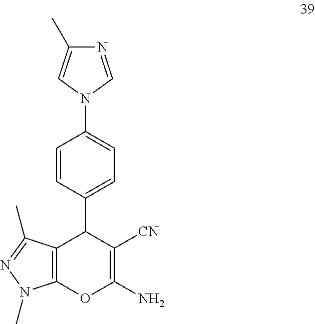 [SMILES: Cc1cn(-c2ccc(C3C(C#N)=C(N)Oc4c3c(C)nn4C)cc2)cn1]

A mixture of malonitrile (128 mg, 1.94 mmol) and 4-(1H-1,2,4-triazol-1-yl)benzaldehyde (363 mg, 1.94 mmol) in 8 mL of anhydrous ethanol was charged with N-methylmorpholine (0.21 mL, 1.94 mmol) for 10 min. To the mixture was added 1,3-dimethyl-1H-pyrazol-5(4H)-one (217 mg, 1.94 mmol) in one portion at room temperature. The reaction mixture was stirred at room temperature for 48 hrs. The reaction mixture was concentrated on rotavapor to dryness and the resulting crude product was purified by Teledyne-Isco flash system by using CH₂Cl₂/MeOH, 0 to 7% of methanol in dichloromethane to provide compound 39 as a viscous solid (300 mg, 45%). ¹H NMR (400 MHz, DMSO-d₆) δ (ppm): 7.65+7.58 (s, 1H), 7.32 (d, J=9.2 Hz, 2H), 7.16 (d, J=8.0 Hz, 2H), 6.92+6.74 (s, 1H), 4.80 (s, 1H), 3.47 (s, 3H), 3.42 (bs, 2H), 2.23 (s, 6H). MS (ESI): Calcd for C19H18N6O: 346, found: 347 (M+H)⁺.

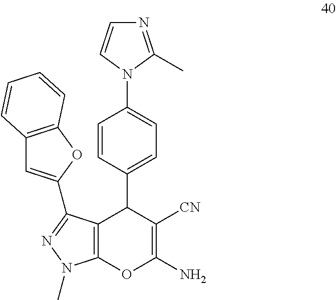 [SMILES: Cc1nccn1-c1ccc(C2C(C#N)=C(N)Oc3c2c(-c2cc4ccccc4o2)nn3C)cc1]

### Example 40

A mixture of malonitrile (109 mg, 1.65 mmol) and 4-(2-methyl-1H-imidazol-1-yl)benzaldehyde (309 mg, 1.65 mmol) in 8 mL of anhydrous ethanol was charged with N-methylmorpholine (0.18 mL, 1.65 mmol) for 10 min. To the mixture was added 3-(benzofuran-2-yl)-1-methyl-1H-pyrazol-5(4H)-one (353 mg, 1.65 mmol) in one portion at room temperature. The reaction mixture was stirred at room temperature for 48 hrs. The reaction mixture was concentrated on rotavapor to dryness and the resulting crude product was purified by Teledyne-Isco flash system by using CH₂Cl₂/MeOH, 0 to 7% of methanol in dichloromethane to provide compound 40 as a viscous brownish solid (100 mg, 45%). ¹H NMR (400 MHz, DMSO-d₆) δ (ppm): 7.59-7.54 (m, 3H), 7.44 (s, 1H), 7.30 (d, J=8.4 Hz, 2H), 7.16 (d, J=8.2 Hz, 2H), 6.98-6.92 (m, 4H), 5.22 (s, 1H), 4.25 (s, 1H), 2.93 (s, 3H), 2.28 (s, 3H). MS (ESI): Calcd for C26H20N6O2: 448, found: 449 (M+H)⁺.

### Example 41

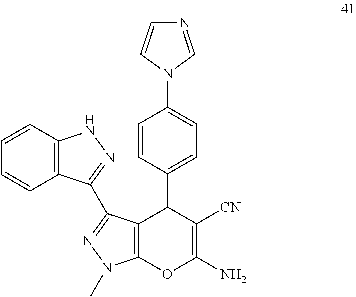 [SMILES: Cn1nc(-c2n[nH]c3ccccc23)c2c1OC(N)=C(C#N)C2c1ccc(-n2ccnc2)cc1]

A mixture of malonitrile (81 mg, 1.22 mmol) and 4-(1H-imidazol-1-yl)benzaldehyde (212 mg, 1 eq) in 8 mL of anhydrous ethanol was charged with N-methylmorpholine (134 uL, 1.0 eq) for 10 mins. To the mixture was added CY572_1 (261 mg, 1.0 eq) in one portion at room temperature. The reaction mixture was stirred at room temperature for 48 hrs. The reaction mixture was concentrated on rotavapor to dryness. The crude product was purified via silica gel column chromatography (MeOH in DCM, 0-8%) to afford the desired product as a viscous solid (100 mg, 45%). ¹H-NMR (400 MHz, d6-DMSO) δ (ppm): 7.92+7.62 (s, 1H), 7.50+7.30 (s, 1H), 7.10-6.74 (m, 12H), 4.45+4.23 (s, 1H), 3.46 (s, 3H). ESI-MS: calcd for C24H19N8O (M+H): 435.2, found: 435.2.

### Example 42

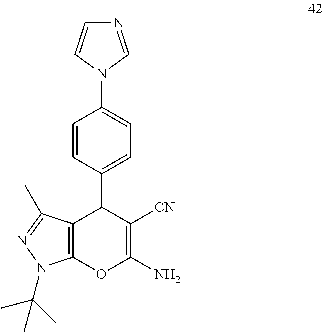 [SMILES: Cc1nn(C(C)(C)C)c2c1C(c1ccc(-n3ccnc3)cc1)C(C#N)=C(N)O2]

A mixture of malonitrile (30.0 mg, 0.45 mmol) and 4-(1H-imidazol-1-yl)benzaldehyde (78.0 mg, 0.45 mmol) in anhydrous ethanol (4.0 mL) was charged with N-methylmorpholine (0.05 mL, 0.45 mmol) for 2 minutes. To the mixture was added 1-(tert-butyl)-3-methyl-1H-pyrazol-5(4H)-one (70.0 mg, 0.45 mmol) in one portion at room temperature. The reaction mixture was stirred at room temperature for 48 hrs. The suspension was filtered under vacuum and off white solid was obtained. The solid was gently washed with hexanes (20 mL) and chilled ethanol (10 mL) and further dried under high vacuum to provide compound 42 as an off white solid (85 mg, 50%). ¹H NMR (400 MHz, DMSO-d₆) δ (ppm): MS (ESI): Calcd for C21H22N6O: 374, found: 375 (M+H)⁺.

### Example 43

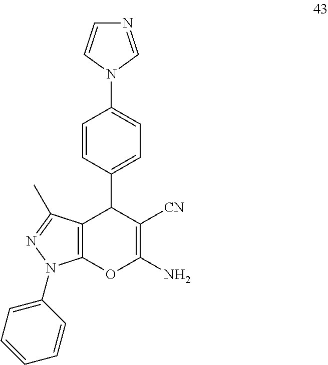 [SMILES: Cc1nn(-c2ccccc2)c2c1C(c1ccc(-n3ccnc3)cc1)C(C#N)=C(N)O2]

A mixture of malonitrile (30.0 mg, 0.45 mmol) and 4-(1H-imidazol-1-yl)benzaldehyde (78.0 mg, 0.45 mmol) in anhydrous ethanol (4.0 mL) was charged with N-methylmorpholine (0.05 mL, 0.45 mmol) for 2 minutes. To the mixture was added 3-methyl-1-phenyl-1H-pyrazol-5(4H)-one (80.0 mg, 0.45 mmol) in one portion at room temperature. The reaction mixture was stirred at room temperature for 48 hrs. The suspension was filtered under vacuum and off white solid was obtained. The solid was gently washed with hexanes (20 mL) and chilled ethanol (10 mL) and further dried under high vacuum to provide compound 43 as an off white solid (120 mg, 66%). ¹H NMR (400 MHz, DMSO-d₆) δ (ppm): 8.25 (s, 1H), 7.79 (d, J=7.6 Hz, 2H), 7.74 (s, 1H), 7.62 (d, J=8.4 Hz, 2H), 7.50 (m, 2H), 7.41-7.33 (m, 4H), 7.26 (s, 1H), 7.10 (s, 1H), 4.78 (s, 1H), 1.83 (s, 3H). MS (ESI): Calcd for C23H18N6O: 394, found: 395 (M+H)⁺.

### Example 44

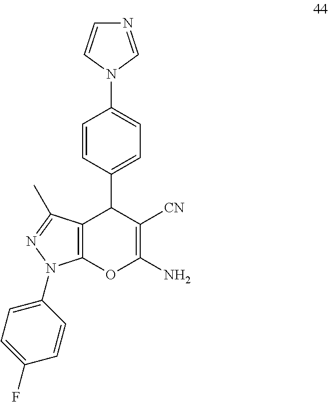 [SMILES: Cc1nn(-c2ccc(F)cc2)c2c1C(c1ccc(-n3ccnc3)cc1)C(C#N)=C(N)O2]

A mixture of malonitrile (30.0 mg, 0.45 mmol) and 4-(1H-imidazol-1-yl)benzaldehyde (78.0 mg, 0.45 mmol) in anhydrous ethanol (4.0 mL) was charged with N-methylmorpholine (0.05 mL, 0.45 mmol) for 2 minutes. To the mixture was added 1-(4-fluorophenyl)-3-methyl-1H-pyrazol-5(4H)-one (90.0 mg, 0.45 mmol) in one portion at room temperature. The reaction mixture was stirred at room temperature for 48 hrs. The suspension was filtered under vacuum and off white solid was obtained. The solid was gently washed with hexanes (20 mL) and chilled ethanol (10 mL) and further dried under high vacuum to provide compound 44 as an off white solid (170 mg, 88%). ¹H NMR (400 MHz, DMSO-d₆) δ (ppm): 8.25 (s, 1H), 7.83-7.74 (m, 3H), 7.62 (d, J=8.4 Hz, 2H), 7.40 (d, J=8.4 Hz, 2H), 7.36-7.26 (m, 4H), 7.10 (s, 1H), 4.77 (s, 1H), 1.82 (s, 3H). MS (ESI): Calcd for C23H17FN6O: 412, found: 413 (M+H)⁺.

### Example 45

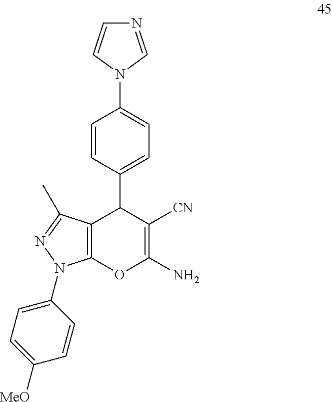 [SMILES: COc1ccc(-n2nc(C)c3c2OC(N)=C(C#N)C3c2ccc(-n3ccnc3)cc2)cc1]

A mixture of malonitrile (30.0 mg, 0.45 mmol) and 4-(1H-imidazol-1-yl)benzaldehyde (78.0 mg, 0.45 mmol) in anhydrous ethanol (4.0 mL) was charged with N-methylmorpholine (0.05 mL, 0.45 mmol) for 2 minutes. To the mixture was added 1-(4-methoxyphenyl)-3-methyl-1H-pyrazol-5(4H)-one (92.0 mg, 0.45 mmol) in one portion at room temperature. The reaction mixture was stirred at room temperature for 48 hrs. The suspension was filtered under vacuum and off white solid was obtained. The solid was gently washed with hexanes (20 mL) and chilled ethanol (10 mL) and further dried under high vacuum to provide compound 45 as an off white solid (160 mg, 84%). ¹H NMR (400 MHz, DMSO-d₆) δ (ppm): 8.24 (s, 1H), 7.74-7.61 (m, 5H), 7.39 (d, J=8.4 Hz, 2H), 7.20 (s, 2H), 7.10 (s, 1H), 7.04 (d, J=9.2 Hz, 2H), 4.76 (s, 1H), 3.81 (s, 3H), 1.81 (s, 3H). MS (ESI): Calcd for C24H20N6O2: 424, found: 425 (M+H)⁺.

### Example 46

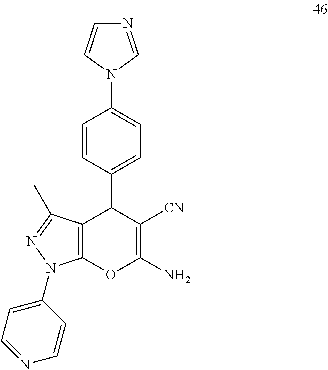 [SMILES: Cc1nn(-c2ccncc2)c2c1C(c1ccc(-n3ccnc3)cc1)C(C#N)=C(N)O2]

A mixture of malonitrile (30.0 mg, 0.45 mmol) and 4-(1H-imidazol-1-yl)benzaldehyde (78.0 mg, 0.45 mmol) in anhydrous ethanol (4.0 mL) was charged with N-methylmorpholine (0.05 mL, 0.45 mmol) for 2 minutes. To the mixture was added 3-methyl-1-(pyridin-4-yl)-1H-pyrazol-5(4H)-one (80.0 mg, 0.45 mmol) in one portion at room temperature. The reaction mixture was stirred at room temperature for 48 hrs. The suspension was filtered under vacuum and off white solid was obtained. The solid was gently washed with hexanes (20 mL) and chilled ethanol (10 mL) and further dried under high vacuum to provide compound 46 as an off white solid (115 mg, 64%). ¹H NMR (400 MHz, DMSO-d₆) δ (ppm): 8.62 (d, J=8.0 Hz, 2H), 8.27 (s, 1H), 7.87 (d, J=5.6 Hz, 2H), 7.75 (s, 1H), 7.63 (d, J=8.4 Hz, 2H), 7.43-7.40 (m, 4H), 7.12 (s, 1H), 4.79 (s, 1H), 1.84 (s, 3H). MS (ESI): Calcd for C22H17N7O: 395, found: 396 (M+H)⁺.

### Example 47

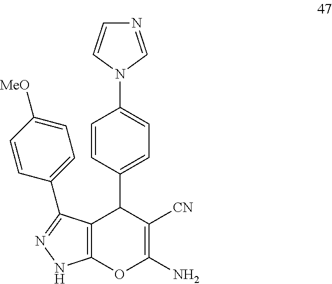 [SMILES: COc1ccc(-c2n[nH]c3c2C(c2ccc(-n4ccnc4)cc2)C(C#N)=C(N)O3)cc1]

A mixture of malonitrile (60.0 mg, 0.90 mmol) and 4-(1H-imidazol-1-yl)benzaldehyde (155.0 mg, 0.90 mmol) in anhydrous ethanol (4.0 mL) was charged with N-methylmorpholine (0.1 mL, 0.90 mmol) for 2 minutes. To the mixture was added 3-(4-methoxyphenyl)-1H-pyrazol-5(4H)-one (170.0 mg, 0.90 mmol) in one portion at room temperature. The reaction mixture was stirred at room temperature for 48 hrs. The suspension was filtered under vacuum and off white solid was obtained. The solid was gently washed with hexanes (20 mL) and chilled ethanol (10 mL) and further dried under high vacuum to provide compound 47 as an off white solid (330 mg, 90%). ¹H NMR (400 MHz, DMSO-d₆) δ (ppm): 12.77 (s, 1H), 8.17 (s, 1H), 7.65 (s, 1H), 7.49 (d, J=8.4 Hz, 2H), 7.42 (d, J=8.4 Hz, 2H), 7.22 (d, J=8.4 Hz, 2H), 7.06 (s, 1H), 6.95 (s, 2H), 6.86 (d, J=8.8 Hz, 2H), 5.05 (s, 1H), 3.71 (s, 3H). MS (ESI): Calcd for C23H18N6O2: 410, found: 411 (M+H)⁺.

### Example 48

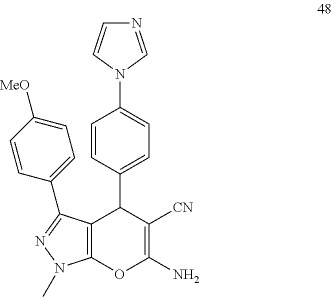 [SMILES: COc1ccc(-c2nn(C)c3c2C(c2ccc(-n4ccnc4)cc2)C(C#N)=C(N)O3)cc1]

A mixture of malonitrile (60.0 mg, 0.90 mmol) and 4-(1H-imidazol-1-yl)benzaldehyde (155.0 mg, 0.90 mmol) in anhydrous ethanol (4.0 mL) was charged with N-methylmorpholine (0.1 mL, 0.90 mmol) for 2 minutes. To the mixture was added 3-(4-methoxyphenyl)-1-methyl-1H-pyrazol-5(4H)-one (185.0 mg, 0.90 mmol) in one portion at room temperature. The reaction mixture was stirred at room temperature for 48 hrs. The suspension was filtered under vacuum and off white solid was obtained. The solid was gently washed with hexanes (20 mL) and chilled ethanol (10 mL) and further dried under high vacuum to provide compound 48 as an off white solid (360 mg, 94%). ¹H NMR (400 MHz, DMSO-d₆) δ (ppm): 8.18 (s, 1H), 7.68 (s, 1H), 7.52-7.46 (m, 4H), 7.27 (d, J=8.0 Hz, 2H), 7.13 (s, 2H), 7.06 (s, 1H), 6.78 (d, J=8.0 Hz, 2H), 5.06 (s, 1H), 3.77 (s, 3H), 3.69 (s, 3H). MS (ESI): Calcd for C24H20N6O2: 424, found: 424 (M+H)⁺.

### Example 49

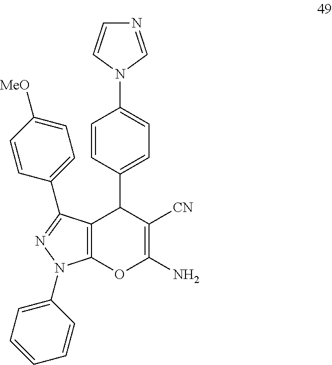 [SMILES: COc1ccc(-c2nn(-c3ccccc3)c3c2C(c2ccc(-n4ccnc4)cc2)C(C#N)=C(N)O3)cc1]

A mixture of malonitrile (60.0 mg, 0.90 mmol) and 4-(1H-imidazol-1-yl)benzaldehyde (155.0 mg, 0.90 mmol) in anhydrous ethanol (4.0 mL) was charged with N-methylmorpholine (0.1 mL, 0.90 mmol) for 2 minutes. To the mixture was added 3-(4-methoxyphenyl)-1-methyl-1H-pyrazol-5(4H)-one (240.0 mg, 0.90 mmol) in one portion at room temperature. The reaction mixture was stirred at room temperature for 48 hrs. The suspension was filtered under vacuum and off white solid was obtained. The solid was gently washed with hexanes (20 mL) and chilled ethanol (10 mL) and further dried under high vacuum to provide compound 49 as an off white solid (420 mg, 96%). ¹H NMR (400 MHz, DMSO-d₆) δ (ppm): 8.18 (s, 1H), 7.90 (d, J=8.8 Hz, 2H), 7.66 (s, 1H), 7.58-7.33 (m, 9H), 7.24 (s, 2H), 7.04 (s, 1H), 6.81 (d, J=8.4 Hz, 2H), 5.15 (s, 1H), 3.69 (s, 3H). MS (ESI): Calcd for C29H22N6O2: 486, found: 487 (M+H)⁺.

### Exemplary Uses of Contemplated Compounds

### Example 50

In vitro growth inhibition of human cancer cells: Growth inhibition of human lung cancer cells by the compounds was measured under anchorage-independent conditions in soft agar. H2122 human lung cancer cells were seeded into 6-well plates (coated with a base layer made of 2 ml of 1% low-melting-point agarose) at 15,000 cells per well in 3 ml of 0.4% low-melting-point agarose containing various concentration of drug. Two weeks after incubation, cells were stained with 1 mg/ml MTT and colonies were counted under a microscope. The IC50 values were defined as the concentration of drug that resulted in 50% reduction in colony number compared to DMSO treated controls. Data are analyzed using Excel (Microsoft), and IC₅₀ values are determined using Prism (Graphpad). The results of the in vitro H2122 growth inhibition activity of the representative compounds of the present invention are shown in the following Table:

| Compound No. | IC50 (μM) |
|---|---|
| 1 | 2.21 |
| 2 | 3.56 |
| 3 | 3.72 |
| 4 | 1.59 |
| 5 | 3.86 |
| 6 | NA |
| 7 | 3.21 |
| 8 | 3.28 |
| 9 | 5.05 |
| 10 | 0.90 |
| 11 | 2.19 |
| 12 | 4.00 |
| 13 | 2.99 |
| 14 | 5.05 |
| 15 | 1.71 |
| 16 | 1.02 |
| 17 | 1.35 |
| 18 | 1.28 |
| 10 | 0.90 |
| 20 | 0.49 |
| 21 | 0.89 |
| 22 | 2.04 |
| 23 | 1.08 |
| 24 | 6.13 |
| 25 | 1.07 |
| 26 | 2.96 |
| 27 | 0.84 |
| 28 | 3.42 |
| 29 | 3.91 |
| 30 | 1.71 |
| 31 | 6.21 |
| 32 | 2.91 |
| 33 | 2.49 |
| 34 | >10 |
| 35 | 2.78 |
| 36 | 8.39 |
| 37 | 6.02 |
| 38 | 5.75 |
| 39 | 5.11 |
| 40 | 1.59 |
| 41 | 1.82 |
| 42 | 1.12 |
| 43 | 0.70 |
| 44 | 0.98 |
| 45 | 1.62 |
| 46 | 2.08 |
| 47 | 1.32 |
| 48 | 1.32 |
| 49 | 3.15 |

### Example 51

ELISA screen: The Elisa screen is based upon the canonical binding principle wherein activated (GTP-bound) protein forms a complex with either RalA or RalB to RalBP1 The ELBA assay was adapted from the widely used Rat activation pull-down assays (Cancer Res. 2005; 65: 7111-7120; WO2013096820 A1) Recombinant GST-His6-RalBP1 fusion protein was purified from bacteria by GST affinity and then adsorbed via a His6 tag directly onto metal-chelate derivatized 96-well microplates. Stably transfected UMUC3 cell lines expressing either FLAG-RalA or FLAG-RalB were created, where the ectopic protein functions as a reporter for Ral activation and the FLAG tag allows highly sensitive and specific detection of the protein. A robust signal to noise ratio (>100:1) using anti-FLAG primary antibody and HRP-conjugated anti-mouse secondary antibody with signal proportional to input protein from 0.3 up to 10 meg of total cell lysate was obtained from cells cultured in 96 well microplates where enough total cell protein can be recovered for analysis. Thereafter, dose response curves were determined for Ral GTPase inhibitors of the invention and RalA GTPase inhibition. Additionally, cell spreading assays following treatment with Ral GTPase inhibitors of the invention in mouse embryonic fibroblasts (MEFs), including a dose response curve for cell spreading in these cells.

Contemplated Pharmaceutical Compositions

The present invention provides compositions of matter that are formulations of one or more active drugs and a pharmaceutically-acceptable carrier. In this regard, the invention provides a composition for administration to a mammalian subject, which may include one or more of the compounds presented herein, or its pharmaceutically acceptable salts.

Pharmaceutically acceptable salts of the compounds of this invention include those derived from pharmaceutically acceptable inorganic and organic acids and bases. Examples of suitable acid salts include acetate, adipate, alginate, aspartate, benzoate, benzenesulfonate, bisulfate, butyrate, citrate, camphorate, camphorsulfonate, cyclopentanepropionate, digluconate, dodecylsulfate, ethanesulfonate, formate, fumarate, glucoheptanoate, glycerophosphate, glycolate, hemisulfate, heptanoate, hexanoate, hydrochloride, hydrobromide, hydroiodide, 2-hydroxyethanesulfonate, lactate, maleate, malonate, methanesulfonate, 2-naphthalenesulfonate, nicotinate, nitrate, oxalate, palmoate, pectinate, persulfate, 3-phenylpropionate, phosphate, picrate, pivalate, propionate, salicylate, succinate, sulfate, tartrate, thiocyanate, tosylate and undecanoate. Other acids, such as oxalic, while not in themselves pharmaceutically acceptable, may be employed in the preparation of salts useful as intermediates in obtaining the compounds of the invention and their pharmaceutically acceptable acid addition salts.

Salts derived from appropriate bases include alkali metal (e.g., sodium and potassium), alkaline earth metal (e.g., magnesium), ammonium and N⁺(C1-4 alkyl)₄ salts. This invention also envisions the quaternization of any basic nitrogen-containing groups of the compounds disclosed herein. Water or oil-soluble or dispersible products may be obtained by such quaternization.

It is generally contemplated that the compounds according to the inventive subject matter may be employed in various therapeutic or prophylactic compositions to affect any condition and/or disease associated with dysfunction (e.g., deviation of activity of at least 10% and more typically at least 20% relative to normal in healthy person) of a Ral GTPase, or where modulation of normal activity is desired for a particular purpose. Thus, and viewed from a different perspective, contemplated compositions may be used for treatment of diseases or conditions where reduction of a Ral GTPase is therapeutically or prophylactically desirable. Therefore, particularly contemplated conditions and diseases include those that are sensitive to changes of Ral GTPase activity. For example, contemplated compounds and compositions may be useful in the prevention and/or treatment of cancer (growth inhibition or reduction of growth of the cancer tissue or cells), and particularly cancer that is associated with dysfunction of Ral GTPase activity, as well as treatment or prevention or reduction of metastasis of a tumor. For example, conditions and diseases to be treated with contemplated compounds and compositions especially include metastatic cancers, particularly metastatic pancreas, prostate, lung, bladder, skin and/or colon cancers.

Depending on the particular purpose, it should also be recognized that contemplated compounds may be combined (in viva, or in a pharmaceutical formulation or administration regimen) with at least one other pharmaceutically active agent to additively or synergistically, provide a therapeutic pr prophylactic effect. Concentrations of second pharmaceutically active ingredients are typically at or preferably below those recommended for stand-alone administration, however, higher concentrations are also deemed suitable for use herein. Most typically, additional pharmaceutical agents include antineoplastic drugs (e.g., angiogenesis inhibitors, antimetabolites, replication inhibitors, drugs targeting DNA repair, proteasome inhibitors, DNA alkylating agents, etc.), immune therapeutic drugs (e.g., modified NK cells, modified T-cells, viral expression systems for delivery of cancer neoepitopes, checkpoint inhibitors, etc.), analgesic drugs, anti-inflammatory drugs, etc.

Therefore, contemplated pharmaceutical compositions will especially include those in which contemplated compounds (and optionally further pharmaceutically active ingredients) are provided with a suitable carrier, wherein contemplated compounds are preferably present at a concentration effective to modulate Ral GTPase signaling in an organism and/or target organ to a degree effective to reduce or prevent cancer growth and/or metastasis.

Depending on the particular use and structure, it is therefore contemplated that the compounds according to the inventive subject matter are present in the composition in an amount between 1 microgram to 1000 milligram, more typically between 10 microgram to 500 milligram, and most typically between 50 microgram to 500 milligram per single dosage unit. Thus, preferred concentrations of contemplated compounds in vivo or in vitro will generally be between 0.1 nM and 100 microM, more typically between 1 nM and 50 microM, and most typically between 10 nM and 10 microM. The recitation of ranges should be interpreted as being inclusive of their endpoints and are intended to serve as a shorthand method of referring individually to each separate value falling within the range. Unless otherwise indicated herein, each individual value is incorporated into the specification as if it were individually recited herein.

The amount of therapeutically active compound that is administered and the dosage regimen for treating a disease condition with the compounds and/or compositions of this invention depends on a variety of factors, including the age, weight, sex, and medical condition of the subject, the severity of the disease, the route and frequency of administration, and the particular compound employed, and thus may vary widely. However, especially suitable quantities are provided above, and may therefore allow for a daily dose of about 0.001 (or even less) to 100 mg/kg body weight, preferably between about 0.01 and about 50 mg/kg body weight and most preferably from about 0.1 to 20 mg/kg body weight. Typically, a daily dose can be administered in one to four doses per day.

In some embodiments, the numbers expressing quantities of ingredients, properties such as concentration, reaction conditions, and so forth, used to describe and claim certain embodiments of the invention are to be understood as being modified in some instances by the term “about.” Accordingly, in some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the invention are approximations, the numerical values set forth in the specific examples are reported as precisely as practicable.

For therapeutic or prophylactic purposes, contemplated compounds are ordinarily combined with one or more excipients appropriate to the indicated route of administration. If administered per os, the compounds may be admixed with lactose, sucrose, starch powder, cellulose esters of alkanoic acids, cellulose alkyl esters, talc, stearic acid, magnesium stearate, magnesium oxide, sodium and calcium salts of phosphoric and sulfuric acids, gelatin, acacia gum, sodium alginate, polyvinylpyrrolidone, and/or polyvinyl alcohol, and then tableted or encapsulated for convenient administration. Such capsules or tablets may contain a controlled-release formulation as may be provided in a dispersion of active compound in hydroxypropylmethyl cellulose. Formulations for parenteral administration may be in the form of aqueous or non-aqueous isotonic sterile injection solutions or suspensions. These solutions and suspensions may be prepared from sterile powders or granules having one or more of the carriers or diluents mentioned for use in the formulations for oral administration. The compounds may be dissolved in water, polyethylene glycol, propylene glycol, ethanol, corn oil, cottonseed oil, peanut oil, sesame oil, benzyl alcohol, sodium chloride, and/or various buffers. Other excipients and modes of administration are well and widely known in the pharmaceutical art.

The compositions of the present invention may be administered orally, parenterally, by inhalation spray, topically, rectally, nasally, buccally, vaginally or via an implanted reservoir. The term “parenteral” as used herein includes subcutaneous, intravenous, intramuscular, intra-articular, intra-synovial, intrasternal, intrathecal, intrahepatic, intralesional and intracranial injection or infusion techniques. Preferably, the compositions are administered orally, intraperitoneally or intravenously.

The pharmaceutically acceptable compositions of this invention may be orally administered in any orally acceptable dosage form including, but not limited to, capsules, tablets, troches, elixirs, suspensions, syrups, wafers, chewing gums, aqueous suspensions or solutions.

The oral compositions may contain additional ingredients such as: a binder such as microcrystalline cellulose, gum tragacanth or gelatin; an excipient such as starch or lactose, a disintegrating agent such as alginic acid, corn starch and the like; a lubricant such as magnesium stearate; a glidant such as colloidal silicon dioxide; and a sweetening agent such as sucrose or saccharin or flavoring agent such as peppermint, methyl salicylate, or orange flavoring. When the dosage unit form is a capsule, it may additionally contain a liquid carrier such as a fatty oil. Other dosage unit forms may contain other various materials which modify the physical form of the dosage unit, such as, for example, a coating. Thus, tablets or pills may be coated with sugar, shellac, or other enteric coating agents. A syrup may contain, in addition to the active ingredients, sucrose as a sweetening agent and certain preservatives, dyes and colorings and flavors. Materials used in preparing these various compositions should be pharmaceutically or veterinarally pure and non-toxic in the amounts used.

For the purposes of parenteral therapeutic administration, the active ingredient may be incorporated into a solution or suspension. The solutions or suspensions may also include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. The parenteral preparation can be enclosed in ampoules, disposable syringes or multiple dose vials made of glass or plastic.

The pharmaceutical forms suitable for injectable use include sterile solutions, dispersions, emulsions, and sterile powders. The final form should be stable under conditions of manufacture and storage. Furthermore, the final pharmaceutical form should be protected against contamination and should, therefore, be able to inhibit the growth of microorganisms such as bacteria or fungi. A single intravenous or intraperitoneal dose can be administered. Alternatively, a slow long-term infusion or multiple short-term daily infusions may be utilized, typically lasting from 1 to 8 days. Alternate day dosing or dosing once every several days may also be utilized.

Sterile, injectable solutions may be prepared by incorporating a compound in the required amount into one or more appropriate solvents to which other ingredients, listed above or known to those skilled in the art, may be added as required. Sterile injectable solutions may be prepared by incorporating the compound in the required amount in the appropriate solvent with various other ingredients as required. Sterilizing procedures, such as filtration, may then follow. Typically, dispersions are made by incorporating the compound into a sterile vehicle which also contains the dispersion medium and the required other ingredients as indicated above. In the case of a sterile powder, the preferred methods include vacuum drying or freeze drying to which any required ingredients are added.

Suitable pharmaceutical carriers include sterile water; saline, dextrose; dextrose in water or saline; condensation products of castor oil and ethylene oxide combining about 30 to about 35 moles of ethylene oxide per mole of castor oil; liquid acid; lower alkanols; oils such as corn oil; peanut oil, sesame oil and the like, with emulsifiers such as mono- or di-glyceride of a fatty acid, or a phosphatide, e.g., lecithin, and the like; glycols; polyalkylene glycols; aqueous media in the presence of a suspending agent, for example, sodium carboxymethylcellulose; sodium alginate; poly(vinylpyrolidone); and the like, alone, or with suitable dispensing agents such as lecithin; polyoxyethylene stearate; and the like. The carrier may also contain adjuvants such as preserving stabilizing, wetting, emulsifying agents and the like together with the penetration enhancer. In all cases, the final form, as noted, must be sterile and should also be able to pass readily through an injection device such as a hollow needle. The proper viscosity may be achieved and maintained by the proper choice of solvents or excipients. Moreover, the use of molecular or particulate coatings such as lecithin, the proper selection of particle size in dispersions, or the use of materials with surfactant properties may be utilized.

U.S. Pat. Nos. 5,916,596, 6,506,405 and 6,537,579 teach the preparation of nanoparticles from the biocompatible polymers, such as albumin Thus, in accordance with the present invention, there are provided methods for the formation of nanoparticles of the present invention by a solvent evaporation technique from an oil-in-water emulsion prepared under conditions of high shear forces (e.g., sonication, high pressure homogenization, or the like).

Alternatively, the pharmaceutically acceptable compositions of this invention may be administered in the form of suppositories for rectal administration. These can be prepared by mixing the agent with a suitable non-irritating excipient that is solid at room temperature but liquid at rectal temperature and therefore will melt in the rectum to release the drug. Such materials include cocoa butter, beeswax and polyethylene glycols.

The pharmaceutically acceptable compositions of this invention may also be administered topically, especially when the target of treatment includes areas or organs readily accessible by topical application, including diseases of the eye, the skin, or the lower intestinal tract. Suitable topical formulations are readily prepared for each of these areas or organs.

Topical application for the lower intestinal tract can be effected in a rectal suppository formulation (see above) or in a suitable enema formulation. Topically-transdermal patches may also be used.

For topical applications, the pharmaceutically acceptable compositions may be formulated in a suitable ointment containing the active component suspended or dissolved in one or more carriers. Carriers for topical administration of the compounds of this invention include, but are not limited to, mineral oil, liquid petrolatum, white petrolatum, propylene glycol, polyoxyethylene, polyoxypropylene compound, emulsifying wax and water. Alternatively, the pharmaceutically acceptable compositions can be formulated in a suitable lotion or cream containing the active components suspended or dissolved in one or more pharmaceutically acceptable carriers. Suitable carriers include, but are not limited to, mineral oil, sorbitan monostearate, polysorbate 60, cetyl esters wax, cetearyl alcohol, 2-octyldodecanol, benzyl alcohol and water.

For ophthalmic use, the pharmaceutically acceptable compositions may be formulated as micronized suspensions in isotonic, pH adjusted sterile saline, or, preferably, as solutions in isotonic, pH adjusted sterile saline, either with or without a preservative such as benzylalkonium chloride. Alternatively, for ophthalmic uses, the pharmaceutically acceptable compositions may be formulated in an ointment such as petrolatum.

The pharmaceutically acceptable compositions of this invention may also be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other conventional solubilizing or dispersing agents.

It should be apparent to those skilled in the art that many more modifications besides those already described are possible without departing from the inventive concepts herein. The inventive subject matter, therefore, is not to be restricted except in the scope of the appended claims. Moreover, in interpreting both the specification and the claims, all terms should be interpreted in the broadest possible manner consistent with the context. In particular, the terms “comprises” and “comprising” should be interpreted as referring to elements, components, or steps in a non-exclusive manner, indicating that the referenced elements, components, or steps may be present, or utilized, or combined with other elements, components, or steps that are not expressly referenced. Where the specification claims refers to at least one of something selected from the group consisting of A, B, C . . . and N, the text should be interpreted as requiring only one element from the group, not A plus N, or B plus N, etc.

1. A compound having a structure according to Formula I or pharmaceutically acceptable enantiomers, tautomers, diastereomers, racemates, and salts thereof 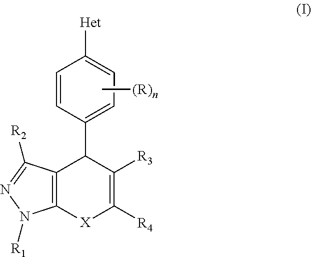
wherein:
R is independently selected from the group consisting of hydrogen, halogen, hydroxy, amino, cyano, —COOH, —SO₂NH₂, oxo, nitro, C₁-C₆ alkyl, C₂-C₆ alkenyl, C₂-C₆ alkynyl, C₃-C₆ cyclalkyl, C₅-C₆ aryl, substituted C₅-C₆ aryl, C₃-C₆ heteroaryl, substituted C₃-C₆ heteroaryl, C₂-C₆ alkoxycarbonyl, CONHSO₂R₅, CONR₅R₆, O—R₅, S—R₅, SO—R₅, SO₂—R₅, NHSO₂R₅, and NHCO₂R₅, and wherein n is an integer between 0 and 4;
R₁ is selected from the group consisting of hydrogen, C₁-C₆ alkyl, C₃-C₆ alkenyl, C₅-C₆ aryl, substituted C₅-C₆ aryl, C₅-C₆ heteroaryl, substituted C₅-C₆ heteroaryl, and C₅-C₁₀ alkylaryl;
R₂ is selected from the group consisting of hydrogen, halogen, amino, CN, COOH, C₁-C₁₀ alkyl, C₁-C₁₀ cycloalkyl, C₂-C₁₀ alkenyl, C₅-C₁₀ aryl, C₅-C₁₀ arylalkyl, substituted C₅-C₆ aryl, optionally substituted C₂-C₁₀ heteroaryl, optionally substituted heterocycloalkyl, optionally substituted heterocycloalkyl fused to aryl, C₁-C₆-alkoxy, C₂-C₆ alkanoyloxy, C₂-C₆ alkanoylamino, C₁-C₆ alkylthio, C₁-C₆ alkylsulfonyl, C₂-C₆ alkoxycarbonyl, CONR₅R₆, O—R₅, NHSO₂R₅ and NHCO₂R₅, wherein the heteroatoms in heteroaryl and heterocycloalkyl are selected from the group consisting of sulfur, nitrogen, and oxygen;
R₃ and R₄ are independently CN, NO₂, NH₂, OH, COOH, CONR₅R₆, NHSO₂R₅, NHCOR₅, or NHCO₂R₅, or together form a 5-membered and 6-membered heterocycle in which the heteroatoms are selected from the group consisting of sulfur, nitrogen, and oxygen;
X is O, NH, or NR₅;
R₅ and R₆ are independently hydrogen, C₁-C₆ alkyl, C₃-C₆ alkenyl, C₅-C₆ aryl, C₂-C₁₀ heteroaryl, substituted C₅-C₁₀ aryl, substituted C₂-C₁₀ heteroaryl, each optionally substituted with one to three groups selected from the group consisting of halogen, C₁-C₆ alkyl, C₁-C₆ alkoxy, C₅-C₆ aryl, and C₃-C₆ heteroaryl, wherein the heteroatom in the heteroaryl is selected from the group consisting of sulfur, nitrogen, and oxygen;
Het is a heteroaryl, optionally substituted with 1 to 4 substituents independently selected from the group consisting of C₁-C₆ alkyl, C₁-C₆ alkoxy, C₂-C₆ alkenyl, C₂-C₆ alkynyl, halogen, hydroxy, amino, amide, cyano, —COOH, —SO₂NH₂, oxo, nitro, alkoxycarbonyl, C₅-C₆ aryl, and C₂-C₆ heteroaryl,
wherein Het has one or more heteroatoms selected from the group consisting of sulfur, nitrogen, and oxygen; and
with the proviso that where X is O, R₃ is CN, R₄ is NH₂, and Het is imidazole, Het is substituted with alkyl or fused with an aryl ring.

2. The compound of claim 1 wherein the compound has structure according to Formula Ia

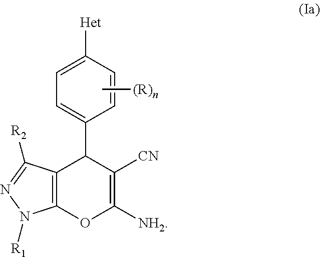

3. The compound of claim 1 wherein the compound has structure according to Formula Ib

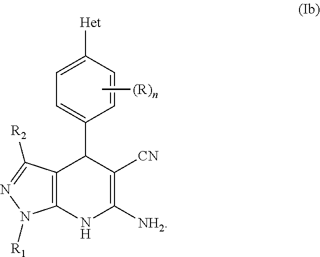

4. The compound of claim 1 wherein the compound has structure according to Formula Ic

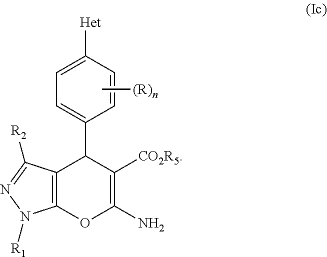

5. The compound of claim 1 wherein the compound has structure according to Formula Id

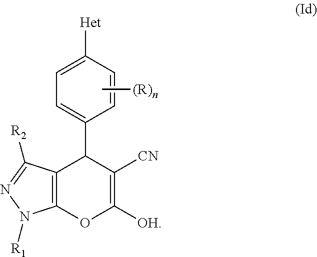

6. The compound of claim 1 wherein the compound has structure according to Formula Ie

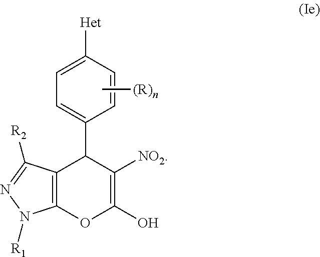

7. The compound of claim 1 wherein Het is

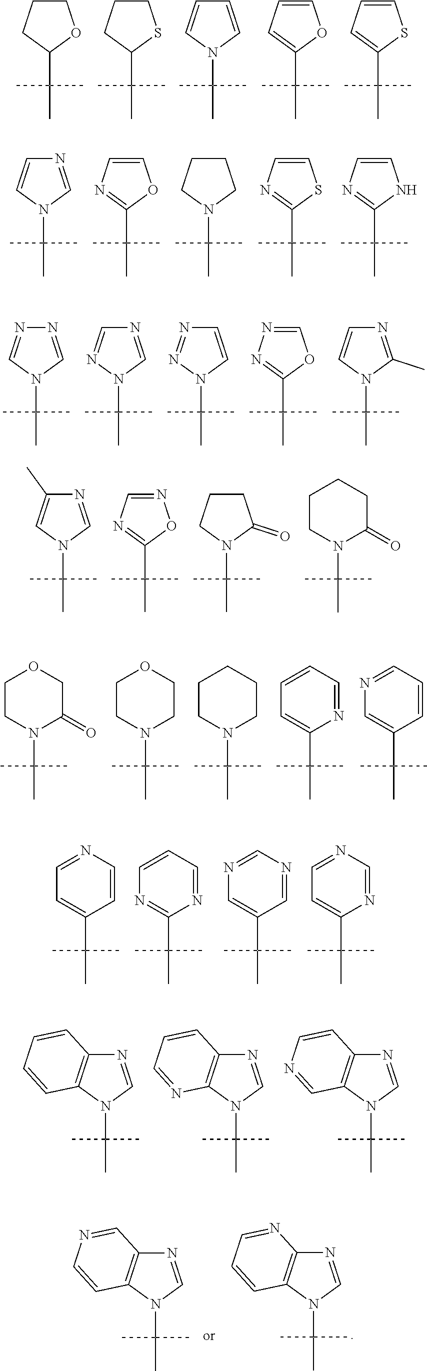

8. The compound of claim 1 wherein Het is a 5- or 6-membered ring with one or two N atoms as heteroatoms.

9. The compound of claim 1 wherein R₁ is hydrogen, C₁-C₆ alkyl, or optionally substituted C₅-C₆ aryl.

10. The compound of claim 1 wherein R₂ is hydrogen, C₁-C₁₀ alkyl, C₁-C₁₀ cycloalkyl, C₅-C₁₀ aryl, substituted C₅-C₆ aryl, optionally substituted C₂-C₁₀ heteroaryl, or optionally substituted heterocycloalkyl.

11. A pharmaceutical composition comprising a compound of claim 1 or a pharmaceutically acceptable enantiomer, tautomer, diastereomer, racemate, or salt thereof, in combination with a pharmaceutically acceptable carrier.

12. The pharmaceutical composition of claim 11 wherein the compound is present in an amount effective to inhibit Ral GTPase in a patient where the composition is administered to the patient.

13. The pharmaceutical composition of claim 11 wherein the compound is present in an amount effective to reduce growth of a cancer in a patient where the composition is administered to the patient.

14. The pharmaceutical composition of claim 11 wherein the compound is present in an amount effective to reduce incidence or multiplicity of metastases of a cancer in a patient where the composition is administered to the patient.

15. The pharmaceutical composition of claim 11 wherein the composition is formulated for oral administration or for injection.

16.-21. (canceled)

22. A method of preventing or treating cancer, comprising a step of administering to an individual in need thereof a therapeutically effective amount of a compound according to claim 1 in an amount effective to inhibit a Ral GTPase in the cancer.

23. The method of claim 22, wherein the compound inhibits at least one of RalA or RalB.

24. The method of claim 22, wherein the cancer is pancreas, prostate, lung, bladder, or colon cancer.

25. The method of claim 24, wherein the cancer is metastatic cancer.

26. A method of preventing or treating metastasis of a cancer in an individual comprising a step of administering to an individual in need thereof a therapeutically effective amount of a compound according to claim 1 in an amount effective to inhibit a Ral GTPase in the cancer.

27. The method of claim 26, wherein the compound inhibits at least one of RalA or RalB.

28. The method of claim 26, wherein the cancer is pancreas, prostate, lung, bladder, or colon cancer.

29. The method of claim 28, wherein the cancer is metastatic cancer.

30. A method of inhibiting at least one of RalA and RalB, comprising a step of contacting RalA and/or RalB with a compound according to claim 1 in an amount effective to inhibit RalA and/or RalB.

31. The method of claim 30 wherein the step of contacting is performed in vivo.

32. The method of claim 30 wherein the amount effective is less than 1 microM.

33. The method of claim 30 wherein inhibition of RalA and/or RalB is inhibition of the GDP-bound forms of RalA and/or RalB.
